(19) United States
(12) Patent Application Publication
(10) Pub. No.: US 2009/0188002 A1
(43) Pub. Date: Jul. 23, 2009

(54) Artificial chromosomes and transchromosomic avians

(75) Inventors: Leandro Christmann, Watkinsville, GA (US); Dawn M. Eberhardt, Danielsville, GA (US); Markley C. Leavitt, Watkinsville, GA (US); Alex J. Harvey, Athens, GA (US)

Correspondence Address: Synageva BioPharma Corp., 111 RIVERBEND ROAD, ATHENS, GA, 30605, US

(73) Assignee: AviGenics, Inc.

(21) Appl. No.: 12/316,591

(22) Filed: Dec. 12, 2008

Related U.S. Application Data
(63) Continuation of application No. 11/362,064, filed on Feb. 24, 2006, now abandoned.
Application No. 11/362,064 is a continuation-in-part of application No. 11/296,119, filed on Dec. 7, 2005, now abandoned.
Application No. 11/296,119 is a continuation-in-part of application No. 11/193,750, filed on Jul. 29, 2005, now abandoned.
Application No. 11/193,750 is a continuation-in-part of application No. 11/068,155, filed on Feb. 28, 2005, now abandoned.
Application No. 11/068,155 is a continuation-in-part of application No. 10/940,315, filed on Sep. 14, 2004, now abandoned.
Application No. 10/940,315 is a continuation-in-part of application No. 10/811,136, filed on Mar. 26, 2004, now abandoned.
Application No. 10/811,136 is a continuation-in-part of application No. 10/790,455, filed on Mar. 1, 2004, now abandoned.
(60) Provisional application No. 60/683,686, filed on May 23, 2005.
(60) Provisional application No. 60/733,669, filed on Nov. 4, 2005.

(51) Int. Cl. A01K 67/027; C12N 15/85

(52) U.S. Cl. 800/19; 800/25

(57) ABSTRACT

The invention includes avians containing an artificial chromosome in their genome and methods of making the avians.

This application is a continuation of U.S. patent application Ser. No. 11/362,064, filed Feb. 24, 2006, the disclosure of which is incorporated in its entirety herein by reference, which is a continuation-in-part of U.S. patent application Ser. No. 11/296,119, filed Dec. 7, 2005, now abandoned, the disclosure of which is incorporated in its entirety herein by reference, and claims the benefit of U.S. provisional application No. 60/683,686, filed May 23, 2005, and U.S. provisional application No. 60/733,669, filed Nov. 4, 2005, the and is a continuation-in-part of U.S. patent application Ser. No. 11/193,750, filed Jul. 29, 2005, now abandoned, the disclosure of which is incorporated in its entirety herein by reference, which is a continuation-in-part of U.S. patent application Ser. No. 11/068,155, filed Feb. 28, 2005, now abandoned, the disclosure of which is incorporated in its entirety herein by reference, and is a continuation-in-part of U.S. patent application Ser. No. 10/940,315, filed Sep. 14, 2004, now abandoned, the disclosure of which is incorporated in its entirety herein by reference, which is a continuation-in-part of U.S. patent application Ser. No. 10/811,136, filed Mar. 26, 2004, now abandoned, the disclosure of which is incorporated in its entirety herein by reference, which is a continuation-in-part of U.S. patent application Ser. No. 10/790,455, filed Mar. 1, 2004, now abandoned, the disclosure of which is incorporated in its entirety herein by reference.

### FIELD OF THE INVENTION

The present invention relates to the field of biotechnology, and more specifically to the field of genome modification. Disclosed herein are compositions including chromosomes and vectors, and methods of use thereof, for the generation of genetically transformed cells and animals including avians.

### BACKGROUND

Transgenic technology to convert animals into “bioreactors” for the production of specific proteins or other substances of pharmaceutical interest (Gordon et al, 1987, Biotechnology 5: 1183-1187; Wilmut et al, 1990, Theriogenology 33: 113-123) offers significant advantages over more conventional methods of protein production by gene expression. For example, recombinant nucleic acid molecules have been engineered and incorporated into transgenic animals so that an expressed heterologous protein may be joined to a protein or peptide that allows secretion of the transgenic expression product into milk or urine, from which the protein may then be recovered.

Another system useful for heterologous protein production is the avian reproductive system. The production of an avian egg begins with formation of a large yolk in the ovary of the hen. The unfertilized oocyte or ovum is positioned on top of the yolk sac. After ovulation the ovum passes into the infundibulum of the oviduct where it is fertilized, if sperm are present, and then moves into the magnum of the oviduct which is lined with tubular gland cells. These cells secrete the egg-white proteins, including ovalbumin, lysozyme, ovomucoid, conalbumin and ovomucin into the lumen of the magnum where they are deposited onto the avian embryo and yolk. The hen oviduct offers outstanding potential as a protein bioreactor because of the high levels of protein production, the promise of proper folding and post-translation modification of the target protein, the ease of product recovery, and the relatively short developmental period of chickens.

One method for creating permanent genomic modification of a eukaryotic cell is to integrate an introduced DNA into an existing chromosome. Retroviruses have so far proven to be the method of choice for efficient integration. However, retroviral integration is directed to a number of insertion sites within the recipient genome so that positional variation in heterologous gene expression can be evident. Unpredictability as to which insertion site is targeted introduces an undesirable lack of control over the procedure. An additional limitation of the use of retroviruses is that the size of the nucleic acid molecule encoding the virus and heterologous sequences may be limited to about 8 kb. In addition, retroviruses may include undesirable features such as splice sites. Although wild-type adeno-associated virus (AAV) often integrates at a specific region in the human genome, replication deficient vectors derived from AAV do not integrate site-specifically possibly due to the deletion of the toxic rep gene. In addition, homologous recombination produces site-specific integration, but the frequency of such integration usually is typically low.

An alternative method for delivering a heterologous nucleic acid into the genome is the use of one or more site-specific enzymes that can catalyze the insertion of nucleic acids into chromosomes. These enzymes recognize relatively short unique nucleic acid sequences that serve for both recognition and recombination. Examples include Cre (Sternberg & Hamilton, 1981, J. Mol. Biol. 150: 467-486, 1981), Flp (Broach et al, 1982, Cell 29: 227-234, 1982) and R (Matsuzaki et al, 1990, J. Bact. 172: 610-618, 1990).

A novel class of phage integrases, that includes the integrase from the phage phiC31, can mediate highly efficient integration of transgenes in mammalian cells both in vitro and in vivo (Thyagarajan et al, Mol. Cell. Biol. 21: 3926-3934, 2001). Constructs and methods of using recombinase to integrate heterologous DNA into a plant, insect or mammalian genome are described by Calos in U.S. Pat. No. 6,632,672, the disclosure of which is incorporated in its entirety herein by reference.

The phiC31 integrase is a member of a subclass of integrases, termed serine recombinases, that include, for example, R4 and TP901-1. Unlike the phage lambda integrases, which belong to a tyrosine class of recombinases, the serine integrases do not require cofactors such as integration host factor. The phiC31 integrase normally mediates integration of the phiC31 bacteriophage into the genome of Streptomyces via recombination between the attP recognition sequence of the phage genome and the attB recognition sequence within the bacterial genome. When a plasmid is equipped with a single attB site, phiC31 integrase will detect and mediate crossover between the attB site and a pseudo-attP site within the mammalian genome. Such pseudo-attP integration sites have now been identified in the mouse and human genomes. If the heterologous DNA is in a circular or supercoiled form, the entire plasmid becomes integrated with attL and attR arms flanking the nucleic acid insert.

Integration mediated by certain integrases, such as PhiC31 integrase-mediated integration, results in the alteration of the recognition or recombination sites themselves so that the integration reaction is irreversible. This will bypass the primary concern inherent with other recombinases, i.e., the reversibility of the integration reaction and excision of the inserted DNA.

Another method for the stable introduction of heterologous nucleic acid (e.g., large heterologous nucleic acids) into a genome is by the use of an artificial chromosome. Artificial chromosomes for expression of heterologous genes in yeast are available, but artificial chromosomes being delivered to avians has not previously been achieved.

Therefore, it is an object of the invention to produce transgenic animals with large nucleic acid segments integrated into their genome and to provide avians which include an artificial chromosome in their genome.

In one useful embodiment, the transgenic avians of the invention can be used to produce polyclonal antibodies to antigens of interest for therapeutic applications. Fully human polyclonal antibodies have proven to be effective therapeutics, and in certain circumstances may be more efficacious than monoclonal antibodies. Polyclonal antibodies, opposed to monoclonal antibodies, are of particular therapeutic value for use against antigenic targets that are either complex in nature, subject to resistance via mutational escape or are highly polymorphic. For example, toxins can require multiple antibodies for effective neutralization. Also, pathogenic virus and bacteria, which can quickly mutate into resistant strains, are targets for polyclonal antibodies. In addition, polyclonal antibodies can be used as a masking therapeutic agent. For example, the polyclonal antibodies may be used in Rh disease therapy and immunosuppressive regimens associated transplant rejection and autoimmune disease.

At present, there are approximately 20 therapeutic polyclonals on the market. Existing polyclonal therapeutics are derived either from animal or human serum which imposes certain drawbacks. For example, polyclonal antibodies have a limited in vivo half-life. In addition, these polyclonals usually cannot be re-administered to a patient due to immune reaction. In addition, human serum derived antibodies, while fully human, have both inherent production limitations as well as certain bio-safety concerns.

Although human polyclonal antibodies have been produced in transgenic mice and cattle (reviewed in, for example, Bruggemann (2004) in Molecular Biology of B cells pp 547-561. Academic Press and Kuroiwa et al (2002) Nature Biotechnol 20:889-894), there are certain limitations to each of these platforms with respect to large-scale manufacture of therapeutic polyclonals. For example, the levels of antibody production achievable in mice is extremely small by virtue of their body size. In cattle, the endogenous immunoglobulin genes are not “knocked-out”, since embryonic stem cell lines necessary for knock-out procedures do not exist. Therefore, contaminating bovine immunoglobulins will be present which will be difficult to separate from human antibodies by standard protein A/G affinity purification procedures. In addition, since the antibodies are produced in animal serum, there are biosafety and serum protein contamination problems.

In order to fully realize the potential of therapeutic polyclonals, a production platform is needed that can efficiently produce large quantities of fully human polyclonal antibody.

Transgenic chickens, which express fully human polyclonal antibodies in response to antigenic stimulation and deposit the antibodies into their eggs, would present such an ideal production system. For example, a single hen has a production capacity of over 250 eggs/year and about 50 to about 100 mg of chicken IgG (also termed IgY) is naturally transported into each egg produced.

The present invention is also directed to methods of producing artificial chromosomes which contain large nucleic acid inserts, such as Ig loci. Producing artificial chromosomes containing a transgene by integrating the transgene into the chromosome can have certain limitations. For example, in some integration methodologies the transgene can integrate into any of the available chromosomes within the cell, including the host cells chromosomes. In certain instances homologous recombination, can overcome this problem. However, homologous recombination has a number of limitations including the requirement that the transgene be specifically engineered for the procedure. In certain useful site specific integration methodologies, the transfected nucleotide sequence must be circular, otherwise integration will introduce a double-stranded break into the artificial chromosome. To by-pass the need for a circular insert the vector can be equipped with two RRSs that flank the desired transgene. However, two recombinases would be required for the integration event and the artificial chromosome would also harbor two recombination sites. The complexity involved in this type of integration would result in an overall low rate of integration. Regardless of the integration methodology employed, the efficiency of integration for large transgenes is typically very much reduced relative to the integration of smaller transgenes, (e.g., up to 1000 fold reduction in efficiency for transgenes over 80 kb (kilobases) relative to smaller transgenes, for example, less than 10 kb). This may be due to certain factors such as the large size of the transgene lowering the rate of transfection. In addition, large transgenes can be susceptible to nicking and breaking due to shear forces and/or nuclease degradation.

One potential difficulty in the use of artificial chromosomes in the production of transchromosomic animals such as avians can be difficulty in preparing a sufficiently homogeneous mixture of artificial chromosomes. Fluorescent synthetic polyamide probes have been used to obtain human chromosomes from their native environment by tagging repeated sequences in the chromosome with labeled polyamides (see, for example, Gygi et al. (2002) Use of fluorescent sequence-specific polyamides to discriminate human chromosomes by microscopy and flow cytometry. Nucleic Acids Res 30: 2790-9).

Purification of artificial chromosomes by methods such as flow cytometry can be limited to only metaphase chromosomes, for example, larger than 60 Mb in size. In certain instances artificial chromosomes which cannot be purified using conventional technologies (e.g., artificial chromosomes less than about 60 Mb is size) could be useful for the production of transchromosomic animals including transchromosomic avians.

What is needed are methods which provide for the efficient introduction of artificial chromosomes into animal genomes such as avian genomes.

### SUMMARY OF THE INVENTION

One useful aspect of the invention relates to methods of producing transchromosomic avians. In one embodiment, the methods include substantially purifying a chromosome followed by introducing the purified chromosome into an avian embryo and thereafter maintaining the embryo under conditions suitable for the embryo to develop and hatch as a chick. In one embodiment, the methods include inserting a heterologous nucleotide sequence into the chromosome before or after substantially purifying the chromosome. In one embodiment, the chromosome is introduced into the avian embryo by microinjection; however, any useful method to introduce the chromosome into the avian embryo is within the scope of the present invention.

It is contemplated that the chromosome may be introduced into the embryo by delivering the chromosome to an avian cell before or after fertilization. For example, the chromosome may be introduced into an ovum or a sperm before fertilization. In another example, the chromosome is introduced into a cell of an embryo (e.g., stage I to stage XII embryo). In one embodiment, the chromosome is introduced into an early stage embryo, for example, and without limitation, a stage I embryo. In one embodiment, the chromosome is introduced into a germinal disc.

The methods provide for the introduction of any useful number of chromosomes into the avian embryo in order to produce a transchromosomal avian. For example, and without limitation, between 1 and about 10,000 chromosomes may be introduced into the embryo. In another example, between 1 and about 1,000 chromosomes may be introduced into the embryo.

The invention also provides for transchromosomal avian cells wherein the artificial chromosome includes a nucleotide sequence which encodes a therapeutic substance. The cells may be isolated from transchromosomal avians and thereafter grown in culture. The invention also contemplates the production of the transchromosomic avian cells by stable introduction of the artificial chromosome into cultured avian cells. Any useful method may be employed for the introduction of the artificial chromosome into the cultured cells including, without limitation, lipofection or microinjection.

The invention also contemplates methods which include isolating an artificial chromosome; introducing the artificial chromosome into an avian embryo; maintaining the embryo under conditions suitable for the embryo to develop and hatch as a chick; and maintaining the chick under conditions suitable to obtain a mature avian wherein the artificial chromosome is present in the genome of the mature avian.

In one aspect, the invention relates to methods which include isolating an artificial chromosomes by flow cytometry. The flow cytometry may be facilitated by a probe which is associated with the artificial chromosome. For example, the probe may be a polyamide probe. In one embodiment, the probe (e.g., polyamide probe) may include a fluorescent molecule or tag.

In one embodiment, the artificial chromosome is present in micronuclei. For example, the artificial chromosome may be present in micronuclei prior to flow cytometry purification of the artificial chromosome. In one embodiment, the micronuclear environment protects the artificial chromosome from degradation or fragmentation that may occur before during or after introduction (e.g., by injection or lipofection) of the artificial chromosome into the avian embryo. In one embodiment, the micronuclei contain diploid mitotic artificial chromosomes. Production of micronuclei can be accomplished by any useful method known in the art, for example, as disclosed in Labidi et al (1987) Experimental Cell Research 617-627, the disclosure of which is incorporated in its entirety herein by reference.

Typically the methods include transferring the embryo, into which the artificial chromosome has been introduced, into a recipient female avian. In one embodiment, the artificial chromosome is an early stage embryo such as a stage I, stage II, stage III, stage IV, stage V or Stage VI embryo. In one useful embodiment, the embryo is a stage I embryo.

In one useful embodiment, the artificial chromosome comprises one or more heterologous recombination sites, for example, between 1 and about 100 recombination sites may be employed. Typically, the chromosome will include a heterologous coding sequence. In one useful embodiment, the heterologous coding sequence consists of or contains a pharmaceutical protein coding sequence. Any useful pharmaceutical protein coding sequence may be employed, such as those disclosed elsewhere herein. In addition, the artificial chromosome can include a promoter, for example, and without limitation, a promoter which functions in tubular gland cells. For example, the promoter may be linked to a pharmaceutical protein coding sequence such that the promoter initiates transcription of the heterologous coding sequence (i.e., the promoter is operably linked to the heterologous coding sequence). The invention also contemplates the inclusion of an IRES (internal ribosome entry site) in the artificial chromosome.

In one aspect, the invention provides for transgenic avians which produce eggs containing polyclonal antibodies, for example, human polyclonal antibodies. The invention also relates to the eggs produced by such an avian. The avians employed in the invention may be any useful avians, such as those avians disclosed elsewhere herein, for example chickens, quail and turkeys. The invention contemplates the production of chimeric birds and germline transgenic birds including G1 and G2 transgenic or transchromosomic avians which produce polyclonal antibodies.

In one useful embodiment of the invention, one or more cells of the transgenic avian contain an artificial chromosome which has coding sequences for a polyclonal antibody. Any useful artificial chromosome may be employed such as those having a centromere selected from the group consisting of an insect centromere, a mammalian centromere and an avian centromere. In one specific embodiment, the artificial chromosome is a satellite artificial chromosome.

The invention also provides for methods of producing artificial chromosomes in cells. In one aspect, methods of the invention include introducing one or more transgenes into an artificial chromosome during assembly of the artificial chromosome. In one useful embodiment, the transgenes contain at least one of a promoter and a coding sequence for a therapeutic protein. In one embodiment, the coding sequence encodes one or more Ig loci such as Igλ, Igκ, IgH, or portions thereof or combinations thereof in its germline. The methods for producing artificial chromosomes containing a transgene are particularly useful for the introduction of large transgenes into the chromosome such as portions of Ig genes, for example, portions of human Ig genes (e.g., an Igλ gene, an Ig H gene and/or an Igκ gene). Certain references which include disclosure that can be useful in certain aspects of the invention include Csonka, et al (2000) Journal of Cell Science 113: 3207-3216 and Nicholson, et al (1999) J. Immunology 163(12):6898-6906. The disclosures of each of these two journal articles are incorporated in their entirety herein by reference.

Integration of a transgene into a defined chromosomal site is useful to improve the predictability of expression of the transgene, which is particularly advantageous when creating transgenic vertebrate animals such as, transgenic avians. Transgenesis by methods that randomly insert a transgene into a genome are often inefficient since the transgene may not be expressed at the desired levels or in desired tissues.

The present invention relates to methods of modifying the genome of vertebrate cells (e.g., production of transgenic vertebrates, in particular, transgenic avians) and to such cells with modified genomes and their progeny. In one embodiment, the methods provide for introducing into vertebrate cells a first recombination site such that the recombination site is inserted into the vertebrate cell genome. Typically, in such embodiments, the genome does not normally include this first recombination site prior to the recombination site introduction. Methods of the invention may also include introducing a nucleotide sequence comprising a second recombination site and a sequence of interest such as a coding sequence into the vertebrate cell or progeny of the vertebrate cell. The nucleotide sequence comprising the second recombination site and the sequence of interest such as a coding sequence may be introduced into the vertebrate cell before, at about the same time as or after the introduction of the first recombination site. Additionally, the present methods may include introducing into the vertebrate cell or progeny cell thereof a substance which facilitates insertion of the nucleotide sequence comprising the second recombination site and the sequence of interest proximal to the first recombination site. For example, the nucleotide sequence comprising the second recombination site and the sequence of interest may be inserted adjacent to or internally in the first recombination site. In one very useful embodiment, the first recombination site and/or the nucleotide sequence comprising the second recombination site and the sequence of interest are stably incorporated into the genome of the cell.

The present invention contemplates the genomic modification of any useful vertebrate cells including, but not limited to, avian cells. Examples of cells which may have their genomes modified in accordance with the present invention include, without limitation, reproductive cells including sperm, ova and embryo cells and nonreproductive cells such as tubular gland cells.

The present invention also relates to methods of producing transgenic vertebrate animals and to the transgenic animals produced by the methods and to their transgenic progeny or descendents. The invention also includes the transgenic cells included in or produced by the transgenic vertebrate animals. Examples of such cells include, without limitation, germline cells, ova, sperm cells and protein producing cells such as tubular gland cells. In one useful embodiment, the transgenic vertebrate animals of the invention are transgenic avians. Transgenic avians of the invention may include, without limitation, chickens, turkeys, ducks, geese, quail, pheasants, parrots, finches, hawks, crows or ratites including ostrich, emu or cassowary.

In accordance with the present invention, methods of producing transgenic vertebrate animals can include introducing into an embryo of a vertebrate animal a first recombination site such that the recombination site is present in sperm or ova of a mature vertebrate animal developed from the embryo. In one useful embodiment, the embryo does not normally include the first recombination site in its genome prior to the recombination site introduction. The methods may also include introducing a nucleotide sequence comprising a second recombination site and a sequence of interest such as a coding sequence into the embryo of the vertebrate animal. The first recombination site and/or the nucleotide sequence comprising the second recombination site and a sequence of interest may be introduced into the embryo of the vertebrate animal before the embryo is fertilized (i.e., when an ovum), at about the same time as introduction of the sperm into the ovum or after fertilization.

The methods can also include introducing the nucleotide sequence comprising a second recombination site and a sequence of interest into an ovum or a sperm of a mature vertebrate animal developed from the embryo (or its descendents) into which the first recombination site was introduced. In one embodiment, the nucleotide sequence comprising a second recombination site and a sequence of interest is introduced into the ovum from the mature vertebrate animal before the ovum is fertilized. In another embodiment, the nucleotide sequence comprising a second recombination site and a sequence of interest is introduced into the ovum at about the time of fertilization. In one particularly useful embodiment, the nucleotide sequence comprising a second recombination site and a sequence of interest is introduced into the ovum after the ovum is fertilized (when an embryo).

The methods may include, upon addition of the nucleotide sequence comprising a second recombination site and a sequence of interest to an embryo, ovum or sperm, introducing into the embryo, ovum or sperm, a substance which facilitates insertion of the nucleotide sequence comprising the second recombination site and the sequence of interest proximal to the first recombination site. For example, the nucleotide sequence comprising the second recombination site and the sequence of interest may be inserted adjacent to or internally in the first recombination site. In one useful embodiment, the methods include introducing into an embryo comprising the first recombination site in its genome, a substance which facilitates insertion of the nucleotide sequence comprising the second recombination site and the sequence of interest proximal to the first recombination site.

In one useful embodiment, these methods include fertilizing an ovum with sperm comprising the first recombination site. The methods can include also introducing into the ovum a nucleotide sequence comprising a second recombination site and a sequence of interest such as a coding sequence and a substance which facilitates insertion of the nucleotide sequence comprising the second recombination site and sequence of interest proximal to (e.g., adjacent to or internally in) the first recombination site. It is contemplated that the nucleotide sequence comprising a second recombination site and a sequence of interest may be introduced into the ovum before or after fertilization by the sperm or at about the same time as fertilization.

In one very useful embodiment of the methods disclosed herein, the nucleotide sequence comprising the second recombination site and the sequence of interest is stably incorporated into the genome of the embryo, ovum or sperm.

The methods disclosed herein typically eventually include exposing a fertilized ovum to conditions which lead to the development of a viable transgenic vertebrate animal.

In one embodiment, the nucleotide sequence of interest includes an expression cassette. Optionally, the nucleotide sequence of interest may include a marker such as, but not limited to, a puromycin resistance gene, a luciferase gene, EGFP-encoding gene, and the like.

Typically, in accordance with methods known in the art or methods disclosed herein, the embryo of the vertebrate animal or fertilized ovum of a mature vertebrate animal of the invention is exposed to conditions which lead to the development of a viable transgenic vertebrate animal.

Embryos that are useful in the present methods include, without limitation, stage I, stage II, stage III, stage IV, stage V, stage VI, stage VII, stage VIII, stage IX, stage X, stage XI and stage XII embryos.

In one embodiment, the nucleotide sequence included with the second recombination site of interest is a coding sequence. The nucleotide sequence of interest included with the second recombination site can be of any useful size. For example, and without limitation, the nucleotide sequence of interest may be from about 0.1 kb to about 10 mb, for example, about 1 kb to about 1 mb. In one embodiment, the nucleotide sequence of interest is about 5 kb to about 5 mb in size, for example, about 5 kb to about 2 mb, e.g., about 8 kb to about 1 mb. In one embodiment, the nucleotide sequence of interest is about 0.5 kb to about 500 kb.

The first recombination site and/or the nucleotide sequence which includes the second recombination site and a sequence of interest such as a coding sequence may be introduced into cells, embryos (i.e., fertilized ova) or sperm by any useful method. These useful methods include, without limitation, cell fusion, lipofection, transfection, microinjection, calcium phosphate co-precipitation, electroporation, protoplast fusion, particle bombardment and the like. In addition, the first recombination site or nucleotide sequence comprising the second recombination site and the sequence of interest may be introduced into cells, embryos, ova or sperm in the presence of a cationic polymer such as PEI and/or other substances disclosed elsewhere herein or known in the art.

In one embodiment, recombination sites employed in the present invention are isolated from bacteriophage and/or bacteria. For example, the recombination sites may be attP sites or attB sites.

The substance which facilitates insertion of the second recombination site and a sequence of interest may be an enzyme. In one embodiment, the substance is a site specific recombinase. In one useful embodiment, the substance which facilitates insertion of the nucleotide sequence is nucleic acid, for example, DNA or RNA. The DNA or RNA may include modified nucleosides as described elsewhere herein or are known to those of skill in the art. In one embodiment, modified nucleosides are employed to extend the half-life of RNA or DNA molecules employed in the present invention. For example, it may be desirable to extend the half life of the RNA or DNA molecules in the presence of a cellular environment. In one useful embodiment, the nucleic acid encodes an enzyme such as a site specific recombinase.

Nonlimiting examples of site specific recombinases which may be employed herein either as protein or encoded by nucleic acid include serine recombinases and tyrosine recombinases. Examples of serine recombinases which may be employed include, without limitation, EcoYBCK, ΦC31, SCH10.38c, SCC88.14, SC8F4.15c, SCD12A.23, Bxb1, WwK, Sau CcrB, Bsu CisB, TP901-1, Φ370.1, Φ105, ΦFC1, A118, Cac1956, Cac1951, Sau CcrA, Spn, TnpX, TndX, SPBc2, SC3C8.24, SC2E1.37, SCD78.04c, R4, ΦRv1, Y4bA and Bja serine recombinases.

In one embodiment of the invention, the present methods include introducing an integration host factor into a cell (e.g., an embryo) to facilitate genomic integration. Such integration host factors may be particularly useful when employing certain substances such as tyrosine recombinases as disclosed herein.

The nucleotide sequence of interest may include a coding sequence. The coding sequence may encode any useful protein. In one useful embodiment, the sequence of interest encodes a pharmaceutical or therapeutic substance. The invention contemplates the production of any useful protein based pharmaceutical or therapeutic substance. Examples of pharmaceutical or therapeutic substances include without limitation at least one of a light chain or a heavy chain of an antibody (e.g., a human antibody) or a cytokine. In one embodiment, the pharmaceutical or therapeutic composition is interferon, erythropoietin, or granulocyte-colony stimulating factor. In one embodiment, the transgenic animal is an avian and the sequence of interest encodes a polypeptide present in eggs produced by the avian.

In one embodiment, integrases such as phage integrases, for example, serine recombinases, such as the integrase from phage phiC31, can mediate the efficient integration of transgenes into target cells both in vitro and in vivo. In one embodiment, when a plasmid is equipped with a single attB site, the integrase detects attP homologous sequences, termed pseudo-attP sites, in a target genome and mediates crossover between the attB site and a pseudo attP site.

In one embodiment, once delivered to a recipient cell, for example, an avian cell, the phiC31 integrase mediates recombination between the att site within the nucleic acid molecule and a bacteriophage attachment site within the genomic DNA of the cell. Both att sites are disrupted and the nucleic acid molecule, with partial att sequences at each end, is stably integrated into the genome attP site. The phiC31 integrase, by disrupting the att sites of the incoming nucleic acid and of the recipient site within the cell genome can preclude any subsequent reverse recombination event that would excise the integrated nucleic acid and reduce the overall efficiency of stable incorporation of the heterologous nucleic acid.

Following delivery of the nucleic acid molecule and a source of integrase activity into a cell population and integrase-mediated recombination, the cells may be returned to an embryo. In the case of avians, late stage blastodermal cells may be returned to a hard shell egg, which is resealed for incubation until hatching. Stage I embryos may be directly microinjected with the polynucleotide and source of integrase activity, isolated, transfected and returned to a stage I embryo which is reimplanted into a hen for further development. Additionally, the transfected cells may be maintained in culture in vitro.

The present invention provides novel methods and recombinant polynucleotide molecules for transfecting and integrating a heterologous nucleic acid molecule into the genome of a cell of a vertebrate animal, such as an avian. Certain methods of the invention provide for the delivery to a cell population a first nucleic acid molecule that comprises a region encoding a recombination site, such as a bacterial recombination site or a bacteriophage recombination site. In one embodiment, a source of integrase activity is also delivered to the cell and can be in the form of an integrase-encoding nucleic acid sequence and its associated promoter or as a region of a second nucleic acid molecule that may be co-delivered with the polynucleotide molecule. Alternatively, integrase protein itself can be delivered directly to the target cell.

The recombinant nucleic acid molecules of the present invention may further comprise a heterologous nucleotide sequence operably linked to a promoter so that the heterologous nucleotide sequence, when integrated into the genomic DNA of a recipient cell, can be expressed to yield a desired polypeptide. The nucleic acid molecule may also include a second transcription initiation site, such as an internal ribosome entry site (IRES), operably linked to a second heterologous polypeptide-encoding region desired to be expressed with the first polypeptide in the same cell.

The present invention provides modified isolated artificial chromosomes useful as vectors to shuttle transgenes or gene clusters into a genome of an avian. By delivery of the modified chromosome to a recipient cell, the target cell, and progeny thereof, become trisomic or transchromosomic. The additional chromosome will typically not affect the subsequent development of the recipient cell and/or embryo, nor interfere with the reproductive capacity of an adult bird developed from such cells or embryos. The chromosome will also be stable within the genome of the cells of the adult bird or within isolated avian cells. The invention provides methods to isolate a population of chromosomes for delivery into embryos or early cells of avians, for example, chickens.

The methods can include inserting a lac-operator sequence into an isolated chromosome and, optionally, inserting a desired transgene sequence within the same chromosome. The lac operator region is typically a concatamer of a plurality of lac operators for the binding of multiple lac repressor molecules. A recombinant DNA molecule is constructed that includes an identified region of the target chromosome, a recombination site such as attB or attP, and the lac-operator concatamer. The recombinant molecule is delivered to an avian cell, and homologous recombination will integrate the heterologous polynucleotide and the lac-operator concatamer into the targeted chromosome. A tag-polypeptide, such as the GPF-lac-repressor fusion protein, binds to the lac-operator sequence for identification and isolation of the genetically modified chromosome. The tagged mitotic chromosome can be isolated using, for instance, flow cytometry.

Among other things, the present invention relates to transchromosomic avians. In a particular aspect, the invention provides for G0 founder transchromosomic avians (e.g., chimeric including, but not limited to, germline chimeric transchromosomic avians) which can give rise to germline transchromosomic offspring, for example, G1 and G2 germline transchromosomic offspring.

Examples of avians which are contemplated for use herein include, without limitation, chicken, turkey, duck, goose, quail, pheasants, parrots, finches, hawks, crows and ratites including ostrich, emu and cassowary.

In one useful aspect, the artificial chromosome employed herein includes a centromere. Any useful centromere may be employed in the present invention including, without limitation, centromeres from insects, mammals or avians.

In one particularly useful embodiment, the artificial chromosomes used herein include a heterologous nucleotide sequence. The nucleotide sequence may be heterologous to the avian and/or heterologous to the artificial chromosome. In one useful embodiment, the heterologous nucleotide sequence includes a coding sequence for a therapeutic substance. In addition, the heterologous nucleotide sequence may include a gene expression controlling region. Any useful gene expression controlling region may be employed in the invention. For example, and without limitation, the gene expression controlling region may include a lysozyme promoter, an ovomucin promoter, a conalbumin promoter, an ovomucoid promoter and/or an ovalbumin promoter or functional portions thereof. See, for example, U.S. patent application Ser. No. 10/114,739, filed Apr. 1, 2002, now issued U.S. Pat. No. 7,199,279; U.S. patent application Ser. No. 10/856,218, filed May 28, 2004, now issued U.S. Pat. No. 7,294,507 and U.S. patent application Ser. No. 10/733,042, filed Dec. 11, 2003. The disclosure of each of these patent applications is incorporated herein by reference in its entirety. In one useful embodiment, the product of the heterologous nucleotide sequence (e.g., therapeutic substance) is delivered to the avian egg (e.g., the egg white) during production of the egg in the avian. The invention also includes the eggs produced by the avians produced by these methods and other methods disclosed herein.

Another aspect of the present invention is a cell, for example, an avian cell, genetically modified with a transgene vector by the methods of the invention. For example, in one embodiment, the transformed cell can be a chicken early stage blastodermal cell or a genetically transformed cell line, including a sustainable cell line. The transfected cell may comprise a transgene stably integrated into the nuclear genome of the recipient cell, thereby replicating with the cell so that each progeny cell receives a copy of the transfected nucleic acid. One useful cell line for the delivery and integration of a transgene comprises a heterologous attP site that can increase the efficiency of integration of a polynucleotide by an integrase, such as phiC31 integrase and, optionally, a region for expressing the integrase.

Another aspect of the present invention is methods of expressing a heterologous polypeptide in a cell by stably transfecting a cell by using site-specific integrase-mediation and a recombinant nucleic acid molecule, as described above, and culturing the transfected cell under conditions suitable for expression of the heterologous polypeptide under the control of a transcriptional regulatory region.

Yet another aspect of the present invention concerns transgenic vertebrate animals, such as birds, for example chickens, comprising a recombinant nucleic acid molecule and which may (though optionally) express a heterologous gene in one or more cells in the animal. For example, in the case of avians, embodiments of the methods for the production of a heterologous polypeptide by the avian tissue involve providing a suitable vector and introducing the vector into embryonic blastodermal cells containing an attP site together with an integrase, for example, a serine recombinase such as phiC31 integrase, so that the vector can integrate into the avian genome at the attP site which has been engineered into the cell genome. A subsequent step may involve deriving a mature transgenic avian from the transgenic blastodermal cells by transferring the transgenic blastodermal cells to an embryo, such as a stage X embryo (e.g., an irradiated stage X embryo), and allowing that embryo to develop fully, so that the cells become incorporated into the bird as the embryo is allowed to develop. In one embodiment, sperm from a G0 bird positive for the transgene is used to inseminate a chicken giving rise to a fully transgenic G1 generation.

One approach may be to transfer a transfected nucleus to an enucleated recipient cell which may then develop into a zygote and ultimately an adult animal. The resulting animal is then grown to maturity.

In the transgenic vertebrate of the present invention, the expression of the transgene may be restricted to specific subsets of cells, tissues or developmental stages utilizing, for example, trans-acting factors acting on the transcriptional regulatory region operably linked to the polypeptide-encoding region of interest of the present invention and which control gene expression in the desired pattern. Tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences. By inserting an integration site such as attP into the genome, it is believed that expression of an integrated coding sequence will be much more predictable.

The invention can be used to express, in large yields and at low cost, a wide range of desired proteins including those used as human and animal pharmaceuticals, diagnostics, and livestock feed additives. Proteins such as growth hormones, cytokines, structural proteins and enzymes including human growth hormone, interferon, lysozyme, and β-casein may be produced by the present methods. In one embodiment, proteins are expressed in the oviduct and deposited in eggs of avians, such as chickens, according to the invention. The present invention includes these eggs and these proteins.

The present invention also includes methods of producing transgenic vertebrate animals, for example, transgenic chickens, which employ the use of integrase, cationic polymers and/nuclear localization signals. The present invention also includes the transgenic vertebrate animals, such as the avians, produced by these methods and other methods disclosed herein. The invention also includes the eggs produced by the transgenic avians produced by these methods and other methods disclosed herein.

In one embodiment, the methods of the invention include introducing into a cell: 1) a nucleic acid comprising a transgene; 2) an integrase activity; and 3) a cationic polymer. Such methods provide for an increased efficiency of transgenic avian production relative to identical methods without the cationic polymer.

In another embodiment, the methods include introducing into a cell: 1) a nucleic acid comprising a transgene; 2) an integrase activity; and 3) a nuclear localization signal. Such methods provide for an increased efficiency of transgenic animal, for example, avian, production relative to identical methods without the nuclear localization signal.

In another embodiment, the methods include introducing into a cell: 1) a nucleic acid comprising a transgene; 2) an integrase activity; 3) a cationic polymer; and 4) a nuclear localization signal. Such methods provide for an increased efficiency of transgenic vertebrate animal production relative to identical methods without the cationic polymer or without the nuclear localization signal.

In one embodiment, the cell is a cell of an embryo, for example, an avian embryo. In one embodiment, the cell is a cell of an early stage avian embryo comprising a germinal disc. The avian cell may be, for example, a cell of a stage I avian embryo, a cell of a stage II avian embryo, a cell of a stage III avian embryo, a cell of a stage IV avian embryo, a cell of a stage V avian embryo, a cell of a stage VI avian embryo, a cell of a stage VII avian embryo, a cell of a stage VIII avian embryo, a cell of a stage IX avian embryo, a cell of a stage X avian embryo, a cell of a stage XI avian embryo or a cell of a stage XII avian embryo. In one particularly useful embodiment, the avian cell is a cell of a stage X avian embryo. In another useful embodiment, the avian cell is a cell of a stage I avian embryo.

The methods provide for the introduction of nucleic acid into the avian cell by any suitable technique known to those of skill in the art. For example, the nucleic acid may be introduced into the avian cell by microinjecting, transfection, electroporation or lipofection. In one particularly useful embodiment, the introduction of the nucleic acid is accomplished by microinjecting.

The nucleic acid which includes a transgene may be DNA or RNA or a combination of RNA and DNA. The nucleic acid may comprise a single strand or may comprise a double strand. The nucleic acid may be a linear nucleic acid or may be an open or closed circular nucleic acid and may be naturally occurring or synthetic.

Integrase activity may be introduced into the cell, such as an avian cell, in any suitable form. In one embodiment, an integrase protein is introduced into the cell. In another embodiment, a nucleic acid encoding an integrase is introduced into the cell. The nucleic acid encoding the integrase may be double stranded DNA, single stranded DNA, double stranded RNA, single stranded RNA or a single or double stranded nucleic acid which includes both RNA and DNA. In one particularly useful embodiment, the nucleic acid is mRNA. Integrase activity may be introduced into the cell by any suitable technique. Suitable techniques include those described herein for introducing the nucleic acid encoding a transgene into a cell. In one useful embodiment, the integrase activity is introduced into the cell with the nucleic acid encoding the transgene. For example, the integrase activity may be introduced into the cell in a mixture with the nucleic acid encoding the transgene.

In one embodiment, a nuclear localization signal (NLS) is associated with the nucleic acid which includes a transgene. For example, the NLS may be associated with the nucleic acid by a chemical bond. Examples of chemical bonds by which an NLS may be associated with the nucleic acid include an ionic bond, a covalent bond, hydrogen bond and Van der Waal's force. In one particularly useful embodiment, the nucleic acid which includes a transgene is associated with an NLS by an ionic bond. NLS may be introduced into the cell by any suitable technique. Suitable techniques included those described herein for introducing the nucleic acid encoding a transgene into a cell. In one useful embodiment, the NLS is introduced into the cell with the nucleic acid encoding the transgene. For example, the NLS may be introduced into the cell while associated with the nucleic acid encoding the transgene.

Cationic polymers may be employed to facilitate the production of transgenic vertebrate animals such as avians. For example, the cationic polymers may, be employed in combination with integrase and/or NLS. Any suitable cationic polymer may be used. For example, and without limitation, one or more of polyethylenimine, polylysine, DEAE-dextran, starburst dendrimers and starburst polyamidoamine dendrimers may be used. In a particularly useful embodiment, the cationic polymer includes polyethylenimine. The cationic polymer may be introduced into the cell by any suitable technique. Suitable techniques included those described herein for introducing the nucleic acid encoding a transgene into a cell. In one useful embodiment, the cationic polymer is introduced into the cell in a mixture with the nucleic acid encoding the transgene. For example, the cationic polymer may be introduced into the avian cell while associated with the nucleic acid encoding the transgene.

In one particularly useful embodiment of the invention, the transgene includes a coding sequence which is expressed in a cell of the transgenic vertebrate animal, for example, a transgenic avian, producing a peptide or a polypeptide (e.g., a protein). The coding sequence may be expressed in any or all of the cells of the transgenic animal. For example, the coding sequence may be expressed in the blood, the magnum and/or the sperm of the animal. In a particularly useful embodiment of the invention, the polypeptide is present in an egg, for example, in the egg white, produce by a transgenic avian.

The present invention also includes methods of dispersing nucleic acid in a cell, for example, in an avian cell (e.g., an avian embryo cell). For example, the nucleic acid may be dispersed in the cytoplasm of a cell. These methods include introducing into a cell a nucleic acid and a dispersing agent, for example, a cationic polymer (e.g., polyethylenimine, polylysine, DEAE-dextran, starburst dendrimers and/or starburst polyamidoamine dendrimers) in an amount that will disperse the nucleic acid in a cell. Typically, the dispersing of the nucleic acid is a homogeneous dispersing. In one embodiment, the dispersed nucleic acid includes a transgene. NLS or integrase activity may also be introduced into the cell. Dispersing of the nucleic acid may be particularly useful when the DNA is introduced into a cell containing a relatively large volume of cytoplasm, such as an avian embryo cell or a germinal disc. Dispersing of the nucleic acid in the cell can increase the likelihood that the nucleic acid will contact and enter the nucleus of the cell into which the nucleic acid has been introduced. Without such dispersing, the nucleic acid may localize to one or more areas within the cell and may not contact the nucleus of the cell. In addition, where the quantity of nucleic acid introduced into the cell is known, dispersing of the nucleic acid can assist in exposing the nucleus in the cell to known or specific concentrations of the nucleic acid.

The methods of the invention include introducing the cell into a recipient animal, for example, an avian such as a chicken, wherein the recipient avian produces an offspring which includes the transgene. The cell may be introduced into a recipient animal by any suitable technique.

The present invention also includes the identification of certain regions in the genome which are advantageous for heterologous gene expression. These regions can be identified by analysis, using methods known in the art, of the transgenic vertebrate animals or cells produced as disclosed herein.

The production of vertebrate animals or avians which are the mature animals developed from the recombinant embryos, ovum and/or sperm of the invention typically are referred to as the G0 generation and are usually hemizygous for each inserted transgene. The G0 generation may be bred to non-transgenic animals to give rise to G1 transgenic offspring which are also hemizygous for the transgene. The G1 hemizygous offspring may be bred to non-transgenic animals giving rise to G2 hemizygous offspring or may be bred together to give rise to G2 offspring homozygous for the transgene. In one embodiment, hemizygotic G2 offspring from the same line can be bred to produce G3 offspring homozygous for the transgene. In one embodiment, hemizygous G0 animals are bred together to give rise to homozygous G1 offspring. These are merely examples of certain useful breeding schemes. The present invention contemplates the employment of any useful breeding scheme such as those known to individuals of ordinary skill in the art.

In one embodiment, the production of transchromosomic avians which are mature avians developed from the recombinant embryos, ovum and/or sperm of the invention typically are referred to as the G0 generation and are usually chimeric for the artificial chromosome. The G0 generation may be bred to non-transgenic (non-transchromosomic) birds to give rise to G1 transchromosomic offspring which contain the artificial chromosome in their genome in all or most cells in the bird. The G1 offspring may in turn be bred to non-transchromosomic birds giving rise to G2 offspring with a single copy of the artificial chromosome in their genome. It is also contemplated that birds which contain the artificial chromosome in their genome in all or most cells (e.g., G1 and/or G2 birds) may be bred together to give rise to offspring containing two of the artificial chromosome in their genome. It is contemplated that this process can be repeated, for example, by crossing the offspring containing two copies of the artificial chromosome in their genome, thus producing birds containing multiple copies, for example, four copies of the artificial chromosome in their genome. It is contemplated that this process can be repeated or modified as would be understood by a practitioner of skill in the art to obtain a bird with a desired number of artificial chromosomes contained in its genome. In one useful embodiment, artificial chromosomes of different types or which contain different nucleotide sequences are individually introduced into the genome of individual avians which are bred to produce an avian containing more than one type of artificial chromosome in its genome.

In one aspect, transchromosomic avians of the invention have a genome which includes a transgene of greater than about 5,000 nucleotides in length. In another aspect, transchromosomic avians of the invention have a genome which includes a transgene of between about 5,000 and about 50,000,000 nucleotides in length. For example, the transgene may be between about 5,000 nucleotides in length and about 5,000,000 nucleotides in length. In one embodiment, the transgene is between about 5,000 nucleotides in length and about 1,000,000 nucleotides in length. For example, the transgene may be between about 5,000 nucleotides in length and about 500,000 nucleotides in length.

In one aspect, transchromosomic avians of the invention have a genome which includes a transgene greater than about 8,000 nucleotides in length. In another aspect, transchromosomic avians of the invention have a genome which includes a transgene of between about 8,000 and about 50,000,000 nucleotides in length. For example, the transgene may be between about 8,000 nucleotides in length and about 5,000,000 nucleotides in length. In one embodiment, the transgene is between about 8,000 nucleotides in length and about 1,000,000 nucleotides in length. For example, the transgene may be between about 8,000 nucleotides in length and about 500,000 nucleotides in length.

In one particularly useful embodiment, the transchromosomic avians of the invention lay eggs which contain one or more heterologous proteins, for example, one or more proteins (e.g., certain pharmaceutical proteins) which are heterologous or exogenous to the egg. The eggs may contain any useful amount of heterologous protein. In one embodiment, the eggs contain the heterologous protein in an amount greater than about 0.01 μg per hard-shell egg. For example, the eggs may contain the heterologous protein in an amount in a range of between about 0.01 μg per hard-shell egg and about 2 grams per hard-shell egg. In one embodiment, the eggs contain between about 0.1 μg per hard-shell egg and about 1 gram per hard-shell egg. For example, the eggs may contain between about 1 μg per hard-shell egg and about 1 gram per hard-shell egg. In one embodiment, the eggs contain between about 1 μg per hard-shell egg and about 1 gram per hard-shell egg. For example, the eggs may contain between about 10 μg per hard-shell egg and about 1 gram per hard-shell egg (e.g., the eggs may contain between about 10 μg per hard-shell egg and about 100 mg per hard-shell egg).

In one useful embodiment, the heterologous protein is present in the egg white of the eggs. In another useful embodiment, the heterologous protein is present in the egg white and is substantially not present in the egg yolk of the eggs.

In one embodiment, the heterologous protein is present in egg white in an amount greater than about 0.01 μg per ml of the egg. In another embodiment, the heterologous protein is present in egg white in an amount in a range of between about 0.01 μg per ml of the egg white and about 0.2 gram per ml of the egg white. For example, the heterologous protein may be present in egg white in an amount in a range of between about 0.1 μg per ml of the egg white and about 0.5 gram per ml of the egg white. In one embodiment, the heterologous protein is present in egg white in an amount in a range of between about 1 μg per ml of the egg white and about 0.2 gram per ml of the egg white. For example, the heterologous protein may be present in egg white in an amount in a range of between about 1 μg per ml of the egg white and about 0.1 gram per ml of the egg white (e.g., the heterologous protein may be present in egg white in an amount in a range of between about 1 μg per ml of the egg white and about 10 mg per ml of the egg white).

Certain publications considered to be useful in the present invention, the disclosures of which are incorporated in their entirety herein by reference, include: Iadonato et al (1996) RARE-cleavage analysis of YACs, Methods Mol Biol 54: 75-85; Popov et al. (1999) A human immunoglobulin lambda locus is similarly well expressed in mice and humans, J Exp Med 189(10): 1611-20; Call et al. (2000) A cre-lox recombination system for the targeted integration of circular yeast artificial chromosomes into embryonic stem cells, Hum Mol Genet. 9(12): 1745-51; Csonka et al. (2000) Novel generation of human satellite DNA-based artificial chromosomes in mammalian cells, Journal of Cell Science 113, 3207-3216; Gogel et al. (1996) Mapping of replication initiation sites in the mouse ribosomal gene cluster, Chromosoma 104, 511-518; Peterson et al. (1998) LCR-dependent gene expression in beta-globin YAC transgenics: detailed structural studies validate functional analysis even in the presence of fragmented YACs, Hum Mol Genet. 7(13): 2079-88; Marschall et al. (1999) Transfer of YACs up to 2.3 mb intact into human cells with polyethylenimine, Gene Ther 6(9): 1634-7; Basu, J., G. Stromberg et al. (2005) Rapid creation of BAC-based human artificial chromosome vectors by transposition with synthetic alpha-satellite arrays, Nucleic Acids Res 33(2): 587-96; Lindenbaum et al. (2004) A mammalian artificial chromosome engineering system (ACE System) applicable to biopharmaceutical protein production, transgenesis and gene-based cell therapy, Nucleic Acids Res 32(21): e172; Nicholson et al. (1999) Antibody repertoires of four- and five-feature translocus mice carrying human immunoglobulin heavy chain and kappa and lambda light chain yeast artificial chromosomes, J Immunol 163(12): 6898-906; Huxley (1994) Genetic Engineering. J. K. Setlow, New York, N.Y., Plenum Press, 16: 65-91; Harvey et al. (2002) Consistent Production of Transgenic Chickens using Replication Deficient Retroviral Vectors and High-throughput Screening Procedures, Poultry Science 81(2): 202-12; Tomizuka et al (1997) Functional expression and germline transmission of a human chromosome fragment in chimeric mice, Nature Genetics 16:133-143; and Williams et al (1993) Cloning and sequencing of human immunoglobulin V-lambda gene segments, Eur J Immunol 23:1456-1461.

Any useful combination of features described herein is included within the scope of the present invention provided that the features included in any such combination are not mutually inconsistent as will be apparent from the context, this specification, and the knowledge of one of ordinary skill in the art. For example, the term transgenic can encompass the term transchromosomal and methodologies useful for transgenic animals (e.g., avians) and cells disclosed herein may also be employed for transchromosomal avians and avian cells.

Additional objects and aspects of the present invention will become more apparent upon review of the detailed description set forth below when taken in conjunction with the accompanying figures, which are briefly described as follows.

### BRIEF DESCRIPTION OF THE FIGURES

FIG. 1 illustrates phage integrase-mediated integration. A plasmid vector bearing the transgene includes the attB recognition sequence for the phage integrase. The vector along with integrase-coding mRNA, a vector expressing the integrase, or the integrase protein itself, are delivered into cells or embryos. The integrase recognizes DNA sequences in the avian genome similar to attP sites, termed pseudo-attP, and mediates recombination between the attB and pseudo-attP sites, resulting in the permanent integration of the transgene into the avian genome.

FIG. 2 illustrates the persistent expression of luciferase from a nucleic acid molecule after phiC31 integrase-mediated integration into chicken cells.

FIG. 3 illustrates the results of a puromycin resistance assay to measure phiC31 integrase-mediated integration into chicken cells.

FIG. 4 illustrates phiC31 integrase-mediated integration into quail cells. Puromycin resistance vectors bearing attB sites were cotransfected with phiC31 integrase, or a control vector, into QT6 cells, a quail fibrosarcoma cell line. One day after transfection, puromycin was added. Puromycin resistant colonies were counted 12 days post-transfection.

FIGS. 5A and 5B illustrate that phiC31 integrase can facilitate multiple integrations per avian cell. A puromycin resistance vector bearing an attB site was cotransfected with an enhanced green fluorescent protein (EGFP) expression vector bearing an attB site, and a phiC31 integrase expression vector. After puromycin selection, many puromycin resistant colonies expressed EGFP in all of their cells.

FIGS. 5A and 5B are the same field of view with EGFP illuminated with ultraviolet light (FIG. 5A) and puromycin resistant colonies photographed in visible light (FIG. 5B). In FIG. 5B, there are 4 puromycin resistant colonies, two of which are juxtaposed at the top. One of these colonies expressed EGFP.

FIG. 6 shows maps of the small vectors used for integrase assays.

FIG. 7 shows integrase promotes efficient integration of large transgenes in avian cells.

FIG. 8 shows maps of large vectors used for integrase assays.

FIGS. 9a and b illustrates the nucleotide sequence of the integrase-expressing plasmid pCMV-3 lint (SEQ ID NO: 1).

FIGS. 10a and b illustrates the nucleotide sequence of the plasmid pCMV-luc-attB (SEQ ID NO: 2).

FIGS. 11a and b illustrates the nucleotide sequence of the plasmid pCMV-luc-attP (SEQ ID NO: 3).

FIGS. 12a and b illustrates the nucleotide sequence of the plasmid pCMV-pur-attB (SEQ ID NO: 4).

FIGS. 13a and b illustrates the nucleotide sequence of the plasmid pCMV-pur-attP (SEQ ID NO: 5).

FIGS. 14a and b illustrates the nucleotide sequence of the plasmid pCMV-EGFP-attB (SEQ ID NO: 6).

FIG. 15a to f illustrates the nucleotide sequence of the plasmid p12.0-lys-LSPIPNMM-CMV-pur-attB (SEQ ID NO: 7).

FIG. 16a to f illustrates the nucleotide sequence of the plasmid pOMIFN-Ins-CMV-pur-attB (SEQ ID NO: 8).

FIGS. 17a and b illustrates the nucleotide sequence of the integrase-expressing plasmid pRSV-Int (SEQ ID NO: 9).

FIGS. 18a and b illustrates the nucleotide sequence of the plasmid pCR-XL-TOPO-CMV-pur-attB (SEQ ID NO: 10).

FIG. 19 illustrates the nucleotide sequence of the attP containing polynucleotide SEQ ID NO: 11.

FIG. 20 illustrates in schematic from the integration of a heterologous att recombination site into an isolated chromosome. The attB sequence is linked to selectable marker such as a puromycin expression cassette and is flanked by sequences found in the target site of the chromosome to be modified. The DNA is transfected into cells containing the chromosome and stable transfectants are selected for by drug resistance. Site specific integration may be confirmed by several techniques including PCR.

FIG. 21 illustrates the persistent expression of luciferase from a nucleic acid molecule after phiC31 integrase-mediated integration into chicken cells bearing a wild-type attP sequence.

FIG. 22 illustrates the distribution of plasmid DNA in a stage I embryo.

FIG. 23 illustrates the distribution of plasmid DNA in a stage I embryo in the presence of low molecular weight polyethylenimine.

FIG. 24 illustrates the distribution of plasmid DNA in a stage I embryo in the presence of low molecular weight polyethylenimine.

FIG. 25 illustrates the integration of a gene of interest (i.e., transgene OMC24-IRES-EPO) into an artificial chromosome by integration (which takes place inside of a host cell) wherein cells containing the recombinant chromosome can be selected for based on hygromycin resistance.

FIG. 26 illustrates the insertion of a nucleotide sequence of interest (A) into an attP site contained in an ALV genome which has been integrated into a chicken chromosome (B). The nucleotide sequence can be introduced into a cell containing the ALV genome by any useful method such as microinjection or transduction. For example, the nucleotide sequence can be introduced into an avian egg or germinal disc at any useful stage of development. For example, the nucleotide sequence can be introduced into a stage X egg by transduction. In another example, the nucleotide sequence can be introduced into a stage I egg by microinjection.

FIG. 27 shows human light-chain locus (27A) and heavy-chain locus (27B) containing YACs. V regions are numbered according to their gene family and their position in the locus, following the system of Lefranc et al (1999) IMCT, the international ImMuunoGenTics database Nucleic Acids Res. 27:209, the disclosure of which is incorporated in its entirety herein by reference. The Ig Heavy YAC contains the complete D and J region loci, the intro enhancer (not marked) and the Igμ and Igδ C regions. The IgLambda YAC contains the seven paired λJ and C regions, four of which are functional, and the 3′ enhancer.

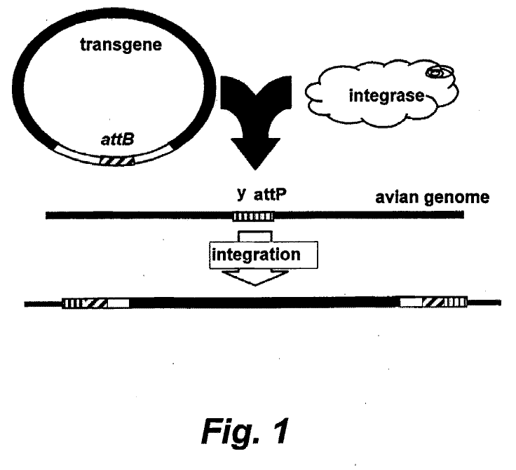

### DEFINITIONS AND ABBREVIATIONS

For convenience, definitions of certain terms and certain abbreviations employed in the specification, examples and appended claims are collected here.

Abbreviations used in the present specification include the following: aa, amino acid(s); bp, base pair(s); kb, kilobase(s); mb, megabase(s); att, bacterial recombination attachment site; IU, infectious units; mg, milligram(s); μg, microgram(s); ml, milliliter(s).

As used in this specification and the appended claims, the singular forms “a,” “an” and “the” include plural references unless the content clearly dictates otherwise. Thus, for example, reference to “an antigen” includes a mixture of two or more such agents.

The term “antibody” as used herein refers to polyclonal and monoclonal antibodies and fragments thereof, and immunologic binding equivalents thereof. Antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

As used herein, an “artificial chromosome” is a nucleic acid molecule that can stably replicate and segregate alongside endogenous chromosomes in a cell. Artificial chromosomes have the capacity to act as gene delivery vehicles by accommodating and expressing foreign genes contained therein. A mammalian artificial chromosome (MAC) refers to chromosomes that have an active mammalian centromere(s). Plant artificial chromosomes, insect artificial chromosomes and avian artificial chromosomes refer to chromosomes that include plant, insect and avian centromeres, respectively. A human artificial chromosome (HAC,) refers to chromosomes that include human centromeres. For exemplary artificial chromosomes, see, for example, U.S. Pat. Nos. 6,025,155, issued Feb. 15, 2000; 6,077,697, issued Jun. 6, 2000; 5,288,625, issued Feb. 22, 1994; 5,712,134, issued Jan. 27, 1998; 5,695,967, issued Dec. 9, 1997; 5,869,294, issued Feb. 9, 1999; 5,891,691, issued Apr. 6, 1999 and 5,721,118, issued Feb. 24, 1998 and published International PCT application Nos., WO 97/40183, published Oct. 30, 1997; WO 98/08964, published Mar. 5, 1998, published U.S. patent application Ser. No. 08/835,682, filed Apr. 10, 1997, now abandoned; 10/151,078, filed May 16, 2002, now abandoned; 10/235,119, filed Sep. 3, 2002, now abandoned; 10/086,745, filed Feb. 28, 2002, now abandoned, the disclosures of which are incorporated herein in their entireties by reference. The term “chromosome” may be used interchangeably with the term “artificial chromosome” as will be apparent based on the context of such use.

Foreign genes that can be contained in artificial chromosome expression systems can include, but are not limited to, nucleic acid that encodes therapeutically effective substances, such as anti-cancer agents, enzymes, hormones and antibodies. Other examples of heterologous DNA include, but are not limited to, DNA that encodes traceable marker proteins (reporter genes), such as fluorescent proteins, such as green, blue or red fluorescent proteins (GFP, BFP and RFP, respectively), other reporter genes, such as beta-galactosidase and proteins that confer drug resistance, such as a gene encoding hygromycin-resistance.

The term “avian” as used herein refers to any species, subspecies or race of organism of the taxonomic class ava, such as, but not limited to chicken, turkey, duck, goose, quail, pheasants, parrots, finches, hawks, crows and ratites including ostrich, emu and cassowary. The term includes the various known strains of Gallus gallus, or chickens, (for example, White Leghorn, Brown Leghorn, Barred-Rock, Sussex, N.H., Rhode Island, Australorp, Minorca, Amrox, California Gray), as well as strains of turkeys, pheasants, quails, duck, ostriches and other poultry commonly bred in commercial quantities. It also includes an individual avian organism in all stages of development, including embryonic and fetal stages. The term “avian” also may denote “pertaining to a bird”, such as “an avian (bird) cell.”

The terms “chimeric animal” or “mosaic animal” are used herein to refer to an animal in which a nucleotide sequence of interest is found in some but not all cells of the animal, or in which the recombinant nucleic acid is expressed, in some but not all cells of the animal. The term “tissue-specific chimeric animal” indicates that the recombinant gene is present and/or expressed in some tissues but not others.

The term “coding region” as used herein refers to a continuous linear arrangement of nucleotides which may be translated into a polypeptide. A full length coding region is translated into a full length protein; that is, a complete protein as would be translated in its natural state absent any post-translational modifications. A full length coding region may also include any leader protein sequence or any other region of the protein that may be excised naturally from the translated protein.

The term “cytokine” as used herein refers to any secreted polypeptide that affects a function of cells and modulates an interaction between cells in the immune, inflammatory or hematopoietic response. A cytokine includes, but is not limited to, monokines and lymphokines. Examples of cytokines include, but are not limited to, interferon α2b, Interleukin-1 (IL-1), Interleukin-6 (IL-6), Interleukin-8 (IL-8), Tumor Necrosis Factor-α (TNF-α) and Tumor Necrosis Factor α (TNF-α).

As used herein, “delivery,” which is used interchangeably with “transfection,” refers to the process by which exogenous nucleic acid molecules are transferred into a cell such that they are located inside the cell.

As used herein, “DNA” is meant to include all types and sizes of DNA molecules including cDNA, plasmids and DNA including modified nucleotides and nucleotide analogs.

The term “expressed” or “expression” as used herein refers to the transcription from a gene to give an RNA nucleic acid molecule at least complementary in part to a region of one of the two nucleic acid strands of the gene. The term “expressed” or “expression” as used herein may also refer to the translation from an RNA molecule to give a protein, a polypeptide or a portion thereof. In one embodiment, for heterologous nucleic acid to be expressed in a host cell, it must initially be delivered into the cell and then, once in the cell, ultimately reside in the nucleus.

The term “gene” or “genes” as used herein refers to nucleic acid sequences that encode genetic information for the synthesis of a whole RNA, a whole protein, or any portion of such whole RNA or whole protein. Genes that are not naturally part of a particular organism's genome are referred to as “foreign genes,” “heterologous genes” or “exogenous genes” and genes that are naturally a part of a particular organism's genome are referred to as “endogenous genes”. The term “gene product” refers to an RNA or protein that is encoded by the gene. “Endogenous gene products” are RNAs or proteins encoded by endogenous genes. “Heterologous gene products” are RNAs or proteins encoded by “foreign, heterologous or exogenous genes” and are, therefore, not naturally expressed in the cell.

As used herein, the terms “heterologous” and “foreign” with reference to nucleic acids, such as DNA and RNA, are used interchangeably and refer to nucleic acid that does not occur naturally as part of a chromosome, a genome or cell in which it is present or which is found in a location(s) and/or in amounts that differ from the location(s) and/or amounts in which it occurs in nature. It can be nucleic acid that is not endogenous to the genome, chromosome or cell and has been exogenously introduced into the genome, chromosome or cell. Examples of heterologous DNA include, but are not limited to, DNA that encodes a gene product or gene product(s) of interest, for example, for production of an encoded protein. Examples of heterologous DNA include, but are not limited to, DNA that encodes traceable marker proteins, DNA that encodes therapeutically effective substances, such as anti-cancer agents, enzymes and hormones and as antibodies. The terms “heterologous” and “exogenous” in general refer to a biomolecule such as a nucleic acid or a protein that is not normally found in a certain cell, tissue or other component contained in or produced by an organism. For example, a protein that is heterologous or exogenous to an egg is a protein that is not normally found in the egg.

The term “immunoglobulin polypeptide” as used herein refers to a constituent polypeptide of an antibody or a polypeptide derived therefrom. An “immunological polypeptide” may be, but is not limited to, an immunological heavy or light chain and may include a variable region, a diversity region, joining region and a constant region or any combination, variant or truncated form thereof. The term “immunological polypeptides” further includes single-chain antibodies comprised of, but not limited to, an immunoglobulin heavy chain variable region, an immunoglobulin light chain variable region and optionally a peptide linker.

The terms “integrase” and “integrase activity” as used herein refer to a nucleic acid recombinase of the serine recombinase family of proteins.

The term “internal ribosome entry sites (IRES)” as used herein refers to a region of a nucleic acid, most typically an RNA molecule, wherein eukaryotic initiation of protein synthesis occurs far downstream of the 5′ end of the RNA molecule. A 43S pre-initiation complex comprising the elf2 protein bound to GTP and Met-tRNAᵢ^{Met}, the 40 ribosomal subunit, and factors elf3 and 31f1A may bind to an “IRES” before locating an AUG start codon. An “IRES” may be used to initiate translation of a second coding region downstream of a first coding region, wherein each coding region is expressed individually, but under the initial control of a single upstream promoter. An “IRES” may be located in a eukaryotic cellular mRNA.

As used herein, the term “large nucleic acid molecules” or “large nucleic acids” refers to a nucleic acid molecule of at least about 0.05 mb in size, greater than 0.5 mb, including nucleic acid molecules at least about 0.6, 0.7, 0.8, 0.9, 1, 5, 10, 30, 50 and 100, 200, 300, 500 mb in size. Large nucleic acid molecules typically can be on the order of about 10 to about 450 or more mb, and can be of various sizes, such as, for example, from about 250 to about 400 mb, about 150 to about 200 mb, about 90 to about 120 mb, about 60 to about 100 mb and about 15 to 50 mb. A large nucleic acid molecule may be larger than about 8 kb (e.g., about 8 kb to about 1 mb) as will be apparent based on the context.

Examples of large nucleic acid molecules include, but are not limited to, natural chromosomes and fragments thereof, especially mammalian chromosomes and fragments thereof which retain a centromere or retain a centromere and telomeres, artificial chromosome expression systems (ACEs which include a mouse centromere; also called satellite DNA-based artificial chromosomes (SATACs); see U.S. Pat. Nos. 6,025,155, issued February 15; and 6,077,697, issued Jun. 20, 2000), mammalian artificial chromosomes (MACs), plant artificial chromosomes, insect artificial chromosomes, avian artificial chromosomes and minichromosomes (see, e.g., U.S. Pat. Nos. 5,712,134, issued Jan. 27, 1998; 5,891,691, issued Apr. 6, 1999; and 5,288,625, issued Feb. 22, 1994). Useful large nucleic acid molecules can include a single copy of a desired nucleic acid fragment encoding a particular nucleotide sequence, such as a gene of interest (transgene of interest), or can carry multiple copies thereof or multiple genes or different heterologous sequences of nucleotides. For example, the chromosomes may carry 1 to about 100 or 1 to about 1000 or even more copies of a gene of interest. Large nucleic acid molecules can be associated with proteins, for example chromosomal proteins, that typically function to regulate gene expression and/or participate in determining overall structure.

A “monoclonal antibody” is an antibody in a population of antibodies each of which have the same primary structure.

“Native” as used herein means being naturally associated with or a substance that is produced by a component or organism of interest (in which case the substance would be native to the component or organism) or being in an original form.

A “nucleic acid fragment of interest” or “nucleotide sequence of interest” may be a trait-producing sequence, by which it is meant a sequence conferring a non-native trait upon the cell in which the protein encoded by the trait-producing sequence is expressed. The term “non-native” when used in the context of a trait-producing sequence means that the trait produced is different than one would find in an unmodified organism which can mean that the organism produces high amounts of a natural substance in comparison to an unmodified organism, or produces a non-natural substance. For example, the genome of a bird could be modified to produce proteins not normally produced in birds such as, for example, useful animal proteins (e.g., human proteins) such as hormones, cytokines and antibodies.

A nucleic acid fragment of interest may additionally be a “marker nucleic acid” or expressed as a “marker polypeptide”. Marker genes encode proteins that can be easily detected in transformed cells and are, therefore, useful in the study of those cells. Examples of suitable marker genes include β-galactosidase, green or yellow fluorescent proteins, enhanced green fluorescent protein, chloramphenicol acetyl transferase, luciferase, and the like. Such regions may also include those 5′ noncoding sequences involved with initiation of transcription and translation, such as the enhancer, TATA box, capping sequence, CAAT sequence, and the like.

As used herein, “nucleic acid” refers to a polynucleotide containing at least two covalently linked nucleotide or nucleotide analog subunits. A nucleic acid can be a deoxyribonucleic acid (DNA), a ribonucleic acid (RNA), or an analog of DNA or RNA. Nucleotide analogs are commercially available and methods of preparing polynucleotides containing such nucleotide analogs are known (Lin et al. (1994) Nucl. Acids Res. 22:5220-5234; Jellinek et al. (1995) Biochemistry 34:11363-11372; Pagratis et al. (1997) Nature Biotechnol. 15:68-73). The nucleic acid can be single-stranded, double-stranded, or a mixture thereof. For purposes herein, unless specified otherwise, the nucleic acid is double-stranded, or if it is apparent from the context that the nucleic acid is not double stranded. Nucleic acids include any natural or synthetic linear and sequential array of nucleotides and nucleosides, for example cDNA, genomic DNA, mRNA, tRNA, oligonucleotides, oligonucleosides and derivatives thereof. For ease of discussion, certain nucleic acids may be collectively referred to herein as “constructs,” “plasmids,” or “vectors.”

Techniques useful for isolating and characterizing the nucleic acids and proteins of the present invention are well known to those of skill in the art and standard molecular biology and biochemical manuals may be consulted to select suitable protocols without undue experimentation. See, for example, Sambrook et al, 1989, “Molecular Cloning: A Laboratory Manual”, 2nd ed., Cold Spring Harbor, the content of which is herein incorporated by reference in its entirety.

A “nucleoside” is conventionally understood by workers of skill in fields related to the present invention as comprising a monosaccharide linked in glycosidic linkage to a purine or pyrimidine base. A “nucleotide” comprises a nucleoside with at least one phosphate group appended, typically at a 3′ or a 5′ position (for pentoses) of the saccharide, but may be at other positions of the saccharide. A nucleotide may be abbreviated herein as “nt.” Nucleotide residues occupy sequential positions in an oligonucleotide or a polynucleotide. Accordingly a modification or derivative of a nucleotide may occur at any sequential position in an oligonucleotide or a polynucleotide. All modified or derivatized oligonucleotides and polynucleotides are encompassed within the invention and fall within the scope of the claims. Modifications or derivatives can occur in the phosphate group, the monosaccharide or the base.

By way of nonlimiting examples, the following descriptions provide certain modified or derivatized nucleotides. The phosphate group may be modified to a thiophosphate or a phosphonate. The phosphate may also be derivatized to include an additional esterified group to form a triester. The monosaccharide may be modified by being, for example, a pentose or a hexose other than a ribose or a deoxyribose. The monosaccharide may also be modified by substituting hydroxyl groups with hydro or amino groups, by esterifying additional hydroxyl groups. The base may be modified as well. Several modified bases occur naturally in various nucleic acids and other modifications may mimic or resemble such naturally occurring modified bases. Nonlimiting examples of modified or derivatized bases include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl) uracil, (acp3)w, and 2,6-diaminopurine. Nucleotides may also be modified to harbor a label. Nucleotides may also bear a fluorescent label or a biotin label.

The term “operably linked” refers to an arrangement of elements wherein the components so described are configured so as to perform their usual function. Control sequences operably linked to a coding sequence are capable of effecting the expression of the coding sequence. The control sequences need not be contiguous with the coding sequence, so long as they function to direct the expression thereof. For example, intervening untranslated yet transcribed sequences can be present between a promoter sequence and the coding sequence and the promoter sequence can still be considered “operably linked” to the coding sequence.

“Therapeutic proteins” or “pharmaceutical proteins” include an amino acid sequence which in whole or in part makes up a drug. In one embodiment, a pharmaceutical composition or therapeutic composition includes one or more pharmaceutical proteins or therapeutic proteins.

The terms “polynucleotide,” “oligonucleotide,” and “nucleic acid sequence” are used interchangeably herein and include, but are not limited to, coding sequences (polynucleotide(s) or nucleic acid sequence(s) which are transcribed and translated into polypeptide in vitro or in vivo when placed under the control of appropriate regulatory or control sequences); control sequences (e.g., translational start and stop codons, promoter sequences, ribosome binding sites, polyadenylation signals, transcription factor binding sites, transcription termination sequences, upstream and downstream regulatory domains, enhancers, silencers, and the like); and regulatory sequences (DNA sequences to which a transcription factor(s) binds and alters the activity of a gene's promoter either positively (induction) or negatively (repression). No limitation as to length or to synthetic origin are suggested by the terms described above.

As used herein the terms “peptide,” “polypeptide” and “protein” refer to a polymer of amino acids in a serial array, linked through peptide bonds. A “peptide” typically is a polymer of at least two to about 30 amino acids linked in a serial array by peptide bonds. The term “polypeptide” includes proteins, protein fragments, protein analogues, oligopeptides and the like. The term “polypeptides” contemplates polypeptides as defined above that are encoded by nucleic acids, produced through recombinant technology (isolated from an appropriate source such as a bird), or synthesized. The term “polypeptides” further contemplates polypeptides as defined above that include chemically modified amino acids or amino acids covalently or noncovalently linked to labeling moieties.

The terms “percent sequence identity” or “percent sequence similarity” as used herein refer to the degree of sequence identity between two nucleic acid sequences or two amino acid sequences as determined using the algorithm of Karlin & Attschul, Proc. Natl. Acad. Sci. 87: 2264-2268 (1990), modified as in Karlin & Attschul, Proc. Natl. Acad. Sci. 90: 5873-5877 (1993). Such an algorithm is incorporated into the NBLAST and XBLAST programs of Attschul et al, 1990, T. Mol. Biol. 215: 403-410. BLAST nucleotide searches are performed with the NBLAST program, score=100, word length=12, to obtain nucleotide sequences homologous to a nucleic acid molecule of the invention. BLAST protein searches are performed with the XBLAST program, score=50, word length=3, to obtain amino acid sequences homologous to a reference polypeptide. To obtain gapped alignments for comparison purposes, Gapped BLAST is utilized as described in Attschul et al, Nucl. Acids Res. 25: 3389-3402 (1997). When utilizing BLAST and Gapped BLAST programs, the default parameters of the respective programs (e.g. XBLAST and NBLAST) are used. Other algorithms, programs and default settings may also be suitable such as, but not only, the GCG-Sequence Analysis Package of the U.K. Human Genome Mapping Project Resource Centre that includes programs for nucleotide or amino acid sequence comparisons. Examples of useful algorithms are FASTA and BESTFIT.

The term “polyclonal antibodies” as used herein refers to a population of antibodies each of which recognize the same antigen or each of which recognize an antigen of a substance which contains one or more antigens.

The term “promoter” as used herein refers to the DNA sequence that determines the site of transcription initiation by an RNA polymerase. A “promoter-proximal element” is a regulatory sequence generally within about 200 base pairs of the transcription start site.

The term “pseudo-recombination site” as used herein refers to a site at which an integrase can facilitate recombination even though the site may not have a sequence identical to the sequence of its wild-type recombination site. For example, a phiC31 integrase and vector carrying a phiC31 wild-type recombination site can be placed into an avian cell. The wild-type recombination sequence aligns itself with a sequence in the avian cell genome and the integrase facilitates a recombination event. When the sequence from the genomic site in the avian cell, where the integration of the vector took place, is examined, the sequence at the genomic site typically has some identity to, but may not be identical with, the wild-type bacterial genome recombination site. The recombination site in the avian cell genome is considered to be a pseudo-recombination site (e.g., a pseudo-attP site) at least because the avian cell is heterologous to the normal phiC31 phage/bacterial cell system. The size of the pseudo-recombination site can be determined through the use of a variety of methods including, but not limited to, (i) sequence alignment comparisons, (ii) secondary structural comparisons, (iii) deletion or point mutation analysis to find the functional limits of the pseudo-recombination site, and (iv) combinations of the foregoing.

The terms “recombinant cell” and “genetically transformed cell” refer to a cell comprising a combination of nucleic acid segments not found in a single cell with each other in nature. A new combination of nucleic acid segments can be introduced into an organism using a wide array of nucleic acid manipulation techniques available to those skilled in the art. The recombinant cell may harbor a vector that is extragenomic, i.e. that does not covalently insert into the cellular genome, including a non-nuclear (e.g. mitochondrial) genome(s). A recombinant cell may further harbor a vector or a portion thereof that is intragenomic, i.e. covalently incorporated within the genome of the recombinant cell.

The term “recombination site” as used herein refers to a polynucleotide stretch comprising a recombination site normally recognized and used by an integrase. For example, λ phage is a temperate bacteriophage that infects E. coli. The phage has one attachment site for recombination (attP) and the E. coli bacterial genome has an attachment site for recombination (attB). Both of these sites are recombination sites for λ integrase. Recombination sites recognized by a particular integrase can be derived from a homologous system and associated with heterologous sequences, for example, the attP site can be placed in other systems to act as a substrate for the integrase.

The terms “recombinant nucleic acid” and “recombinant DNA” as used herein refer to combinations of at least two nucleic acid sequences that are not naturally found in a eukaryotic or prokaryotic cell. The nucleic acid sequences may include, but are not limited to, nucleic acid vectors, gene expression regulatory elements, origins of replication, suitable gene sequences that when expressed confer antibiotic resistance, protein-encoding sequences and the like. The term “recombinant polypeptide” is meant to include a polypeptide produced by recombinant DNA techniques. A recombinant polypeptide may be distinct from a naturally occurring polypeptide either in its location, purity or structure. Generally, a recombinant polypeptide will be present in a cell in an amount different from that normally observed in nature.

As used herein, the term “satellite DNA-based artificial chromosome (SATAC)” (e.g., ACE) is a type of artificial chromosome. These artificial chromosomes are substantially all neutral non-coding sequences (heterochromatin) except for foreign heterologous, typically gene-encoding nucleic acid, that is present within (see U.S. Pat. Nos. 6,025,155, issued Feb. 15, 2000 and 6,077,697, issued Jun. 20, 2000 and International PCT application No. WO 97/40183, published Oct. 30, 1997).

The term “source of integrase activity” as used herein refers to a polypeptide or multimeric protein having serine recombinase (integrase) activity in an avian cell. The term may further refer to a polynucleotide encoding the serine recombinase, such as an mRNA, an expression vector, a gene or isolated gene that may be expressed as the recombinase-specific polypeptide or protein.

As used herein the term “therapeutic substance” refers to a component that comprises a substance which can provide for a therapeutic effect, for example, a therapeutic protein.

“Transchromosomic avian” means an avian which contains an artificial chromosome in some or all of its cells. A transchromosomic avian can include the artificial chromosome in its germ cells.

The term “transcription regulatory sequences” as used herein refers to nucleotide sequences that are associated with a gene nucleic acid sequence and which regulate the transcriptional expression of the gene. Exemplary transcription regulatory sequences include enhancer elements, hormone response elements, steroid response elements, negative regulatory elements, and the like.

The term “transfection” as used herein refers to the process of inserting a nucleic acid into a host cell. Many techniques are well known to those skilled in the art to facilitate transfection of a nucleic acid into an eukaryotic cell. These methods include, for instance, treating the cells with high concentrations of salt such as a calcium or magnesium salt, an electric field, detergent, or liposome mediated transfection, to render the host cell competent for the uptake of the nucleic acid molecules, and by such methods as micro-injection into a pro-nucleus, sperm-mediated and restriction-mediated integration.

The term “transformed” as used herein refers to a heritable alteration in a cell resulting from the uptake of a heterologous DNA.

As used herein, the term “transgene” means a nucleic acid sequence that is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout).

As used herein, a “transgenic avian” is any avian, as defined herein, in which one or more of the cells of the avian contain heterologous nucleic acid introduced by manipulation, such as by transgenic techniques. The nucleic acid may be introduced into a cell, directly or indirectly, by introduction into a precursor of the cell by way of deliberate genetic manipulation, such as by microinjection or by infection with a recombinant virus. Genetic manipulation also includes classical cross-breeding, or in vitro fertilization. The heterologous nucleic acid may be an artificial chromosome or may be integrated within a chromosome of the avian, or it may be extrachromosomally replicating DNA.

The term “trisomic” as used herein refers to a cell or animal, such as an avian cell or bird that has a 2n+1 chromosomal complement, where n is the haploid number of chromosomes, for the animal species concerned.

The terms “vector” or “nucleic acid vector” as used herein refer to a natural or synthetic single or double stranded plasmid or viral nucleic acid molecule (RNA or DNA) that can be transfected or transformed into cells and replicate independently of, or within, the host cell genome. The term “expression vector” as used herein refers to a nucleic acid vector that comprises a transcription regulatory region operably linked to a site wherein is, or can be, inserted, a nucleotide sequence to be transcribed and, optionally, to be expressed, for instance, but not limited to, a sequence coding at least one polypeptide.

### DETAILED DESCRIPTION

The present invention provides for the production of polyclonal antibodies, for example, human polyclonal antibodies, in avians and isolated avian cells. Such avians or avian cells may produce any useful type of antibody including, but not limited to, one or more of IgG, IgM, IgA, IgE, and IgD including each of the subtypes of these antibodies. For example, subtypes of IgG include IgG1, IgG2, IgG3 and IgG4.

In one particularly useful embodiment, the invention provides for the production of polyclonal antibodies which are deposited in the eggs, of avians, such as chickens. It has been shown that active deposition of chicken IgG into the egg is mediated by specific sequences on the Fc portion of the antibody (Morrison et al (2002) Mol. Immunol. 8:619-25). The IgG Fc antibody portion has also been shown to mediate the deposition into an egg of either intravenously injected human IgG, or human monoclonal antibody produced in vivo from a transplanted chicken B-cell line, with high efficiency (Mohammed et al (1998) Immunotechnology, 4(2):115-25). Chicken IgY does not bind to protein A or G and therefore human IgG can be easily affinity purified from other proteins including chicken-immunoglobulins using protein A and/or protein G based purification methodologies as is known in the art. In a particular aspect, the antibody is deposited in the yolk of the egg of the avian.

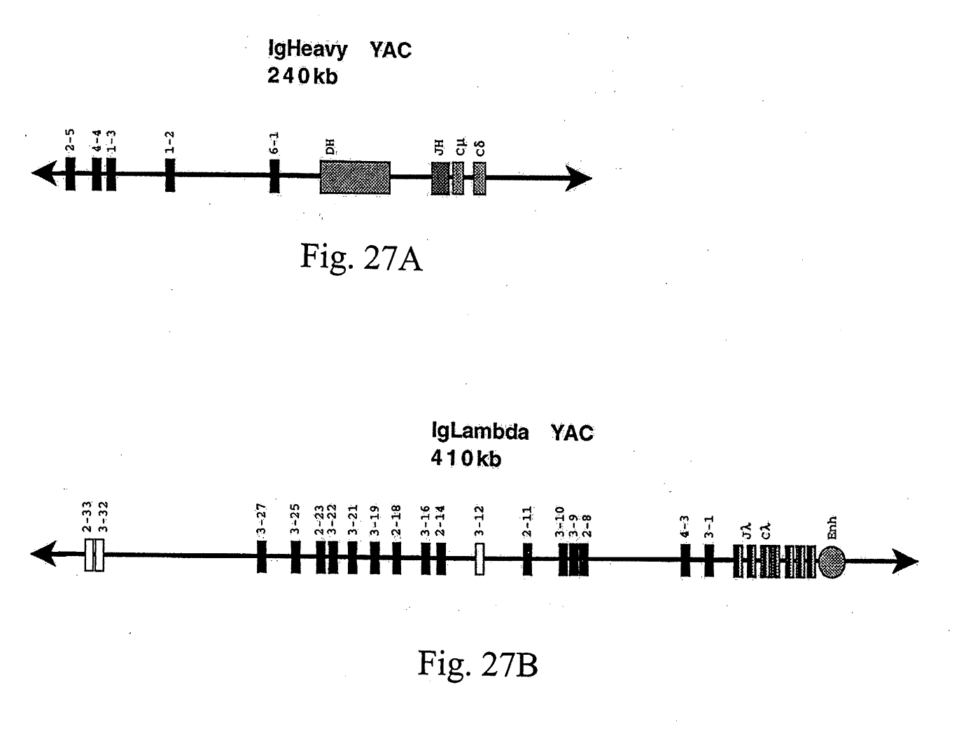

The invention provides for the insertion of large DNA segments into the germline of avians or avian cells. In one particular aspect, the large DNA segments include regions encoding components necessary for the production of human polyclonal antibodies. In one embodiment, the DNA segments include one or more Ig loci. The Ig loci may include one or more of human Igλ, Igκ, IgH, and portions thereof. The Ig loci may be modified to include additional components, such as additional variable or constant regions, or they may be in their native form. Certain Ig loci and other disclosure which may be useful in accordance with the present invention are disclosed in, for example, US patent application publication No. 2002/0132373, published Sep. 19, 2002; US patent application publication No. 2002/0088016, published Jul. 4, 2002; US patent application publication No. 2004/0231012, published Nov. 18, 2004; U.S. Pat. No. 6,348,349, issued Feb. 19, 2002; U.S. Pat. No. 5,545,807, issued Aug. 13, 1996; and Popov et al (1999) J. Exp. Med. 189: 1611-1619. The disclosures of each of these three published patent applications and two issued patents and journal article are incorporated in their entirety herein by reference. In one useful embodiment, the Ig loci shown in FIGS. 27A and 27B are used to produce polyclonal antibodies in accordance with the present invention. These loci are disclosed in Nicholson, et al (1999) J. Immunology 163(12):6898-6906.

The DNA segments comprising regions encoding components necessary for the production of human polyclonal antibodies may be employed in the invention in any useful form. For example, the DNA may be linear or circular. Typically, the DNA segments are present in a cloning vehicle which will facilitate the germline transmission of the DNA encoding the polyclonal antibodies. In one embodiment, artificial chromosomes which include one or more transgenes comprising components necessary for the production of human polyclonal antibodies are contemplated for use to produce germline transgenic avians of the invention. Typically, in this embodiment, a germline chimeric avian is obtained from embryos or germline cells of avians, such as chickens, into which one or more artificial chromosomes comprising the polyclonal antibody transgenes have been introduced as disclosed herein. Subsequently, a transgenic or fully transgenic (e.g., transchromosomic) G1 bird can be obtained from the germline chimera.

In one useful embodiment, one or more Ig loci are included in an artificial chromosome. The artificial chromosome is introduced into an avian genome as disclosed herein.

In one embodiment two artificial chromosomes are used, one having an Ig heavy chain locus and the other having an Ig light chain locus. In one embodiment, the two artificial chromosomes are co-introduced into an avian embryo to produce a germline-transgenic or transchromosomic avian which contains both chromosomes in its genome.

In another embodiment, one or more DNA segments comprising regions necessary for the production of human polyclonal antibodies (e.g., Ig loci) may be used to produce chimeric and germline transgenic avians by incorporation into the genome of an avian by employing integrase mediated transgenesis as disclosed herein.

The invention also contemplates one or more transgenes comprising components necessary for the production of human polyclonal antibodies such as Ig loci being introduced into an immortalized avian cell line, the cells of which may be capable of secreting the polyclonal antibodies into growth medium. In one particular embodiment, immortalized cell lines are derived from tumor cells of an avian oviduct or tumor cells from other cells of an avian, for example, cell lines disclosed in U.S. patent Ser. No. 10/926,707, filed Aug. 25, 2004, now abandoned.

The invention also provides for the production and isolation of cell lines capable of producing monoclonal antibodies. By using standard methodologies well known in the art such as those disclosed in Michael et al (1998) Proc Natl Acad Sci USA 95:1166-1171, the disclosure of which is incorporated in its entirety herein by reference, cells of transgenic avians which contain human Ig heavy chain and human light chain producing loci in their genomes can be used to produce cell lines capable of producing human monoclonal antibodies. For example, the transgenic or transchromosomic chicken is immunized with an antigen and hybridomas are produced by fusing cells (e.g., spleen cells) of the transgenic bird to an immortalized cell line to produce hybridomas. Antibody produced by individual hybridoma clones is screened to identify antibody with binding specificity for the antigen. The exon DNA (e.g., cDNA) encoding the antibody is cloned into mammalian Ig expression vectors which are co-transfected into mammalian myeloma cells to produce antigen specific antibody.

Further, it is contemplated that immunoglobulin genes and other useful products can be provided by the invention. For example, genes encoding monoclonal antibodies can be obtained from monoclonal antibody producing cell lines produced in accordance with the present invention.

The present invention contemplates the production of artificial chromosomes containing large transgenes. In one specific embodiment, the invention provides for the production of artificial chromosomes containing yeast artificial chromosomes (YACs) which contain a large DNA insert such as an Ig locus.

In one embodiment, the present invention provides for the production of artificial chromosomes which contain transgenes wherein the transgene is introduced into the artificial chromosome during the de novo construction of the artificial chromosome. In one particularly useful embodiment of the invention, production of artificial chromosomes which contain large transgenes (e.g., one or more Ig locus) is provided for. Large transgenes as disclosed herein can refer to transgenes greater in size than, for example, about 8 kb or about 10 kb or about 20 kb (e.g., about 8 kb to about 100 mb in size or about 10 kb to about 100 mb in size).

In one embodiment, the invention provides for the introduction of transgene DNA into a cell in which the artificial chromosome is produced at the time of production or assembly of the artificial chromosome. For example, components useful for the production of an artificial chromosome and one or more transgenes are introduced into the cell at about the same time leading to the production of an artificial chromosome containing the transgene or transgenes. In one embodiment, the transgene will include a cloning vector such as a BAC or YAC to which the transgene is linked when introduced into the cell. In another embodiment, the transgene is removed from the cloning vehicle before introduction into the cell. For example, a cloning vehicle containing the transgene can be digested with a nuclease such as a restriction enzyme to release from the cloning vehicle the DNA sequence (transgene) to be included in the artificial chromosome during its assembly. In one embodiment, after restriction nuclease digestion the DNA sequence of interest is separated from the cloning vehicle (e.g., by electrophoresis) prior to introduction of the DNA sequence into the cell. Without wishing to limit the invention to any theory or mechanism of operation, it is believed that as the artificial chromosome is assembled in the cell the transgene(s) is incorporated into the artificial chromosome during the assembly.

In one embodiment, for artificial chromosome assembly, cells may be cotransfected with the transgene DNA and ribosomal RNA encoding DNA (rDNA). In one embodiment, the rDNA is included in a cloning vehicle such as a plasmid or a cosmid. In one useful aspect of the invention, the cell in which the artificial chromosome is produced provides for certain components which will make up the new artificial chromosome such as telomeric nucleotide sequences. The cells which contain the new transgene containing artificial chromosome are identified and isolated. In one embodiment, the transgene carries a selectable marker such as a drug resistant gene providing for the selection of cells containing the new artificial chromosome.

Spontaneous generation of artificial chromosomes may be accomplished by the introduction of heterologous DNA and a marker gene into a cell such as a fibroblast cell, for example, DF-1 cells (U.S. Pat. No. 5,672,485, issued Sep. 30, 1997) or chicken embryo fibroblast cells. However, the methods are not limited to use of a fibroblast cell and the invention contemplates the employment of any useful cell. For example, cell lines such as CHO cells, Hela cells and other animal cell lines, for example, mammalian cell lines, are contemplated for use as disclosed herein. In one embodiment, the present invention contemplates the introduction of a desired transgene into a cell in combination with a marker and heterologous DNA thereby providing for the spontaneous generation of artificial chromosomes containing the desired transgene. The desired transgene typically includes a pharmaceutical protein coding sequence, such as a coding sequence for a pharmaceutical protein disclosed herein, and/or a promoter which functions in the avian oviduct or an active portion thereof. In one useful embodiment, the desired transgene comprises one or more human Ig locus or a portion thereof.

Any useful method for the spontaneous assembly or production of artificial chromosomes is contemplated for use in accordance with the present invention. That is, incorporation of a nucleotide sequence of interest such as a promoter (e.g., ovalbumin promoter, ovomucoid promoter, lysozyme promoter or other promoters which function in the avian oviduct) and/or a coding sequence for a pharmaceutical protein during assembly of the chromosome (e.g., spontaneous assembly) is contemplated. For example, spontaneous assembly of artificial chromosomes (e.g., dicentric chromosomes minichromosomes, satellite artificial chromosomes or megachromosomes) as disclosed in, for example, U.S. Pat. No. 6,743,967, issued Jun. 1, 2004; U.S. Pat. No. 5,288,625, issued Feb. 22, 1994; and WO97/40183, the disclosures of which are incorporated in their entirety herein by reference, is contemplated for use in conjunction with the present invention.

A selectable marker may be included in one or more vectors which are used in artificial chromosome construction (e.g., transgene containing vectors and/or other vectors containing DNA useful in production of the artificial chromosomes, for example, and without limitation, rDNA). In the case where multiple vectors are introduced into a cell to produce an artificial chromosome, some or all of the vectors may have a selectable marker. In such a case, the selectable markers may be different selectable markers. Examples of useful selectable markers include, without limitation, genes which provide for resistance to hygromycin, zeomycin, neomycin and blastomycin. In one embodiment, vectors, for example, linearized vectors, when present in a cell that is producing a chromosome of the invention, may incorporate efficiently into the new chromosome, thereby precluding the need for one or more markers.

One advantage of introducing large DNA molecules into an artificial chromosome during its assembly is that large DNA molecules can be gel purified and directly transfected as a linear molecule into the cell line in which the chromosome is being assembled. Gel purification is important for isolating DNA molecules such as YACs from the other components of the host cells including the native cellular chromosomal DNA. Large, linear YACs are routinely purified in intact form by gel purification methods. Large circular YACs (cYAC) are not able to migrate through agarose in pulsed field gel electrophoresis (PFGE) (i.e, the cYACs remain in the wells) and therefore cannot be gel purified.

The present methods are contemplated for the production of artificial chromosomes which contain any useful transgene. In one embodiment, artificial chromosomes which contain immunoglobulin genes (e.g., coding sequences for immunoglobulins and/or certain native gene expression controlling regions for immunoglobulins), such as human immunoglobulin loci or loci portions, are produced. In one particularly useful embodiment, the Ig loci include coding sequences for the immunoglobulins and certain native gene expression controlling regions of immunoglobulins. The human Ig containing artificial chromosome may be introduced into an avian such as a chicken such that the chicken produces human antibodies in its serum and the antibodies localize to the egg. In one useful embodiment, the antibodies are polyclonal in nature and are produced by immunization of the transgenic animal with an antigen. In the case of such transgenic avians, such as chickens, the invention contemplates the polyclonal human antibodies being deposited in the yolk of laying hens through a native transport system that has been shown to transfer antibodies, including human antibodies, from the blood serum to the yolk of forming eggs. In one embodiment, the invention contemplates the deposition of an amount between about 0.1 μg and about 1 gram of polyclonal antibody per egg.

Human Ig genes are encoded on separate loci. Human heavy chain (IgH) is believed to be encoded by a single locus that is ˜1.5 mb in size. There are believed to be two loci for the human light chain, Igκ and Igλ, either of which may be used for production of functional antibodies. The Igκ, locus is believed to be ˜1.1 mb and the Igλ locus is believed to be ˜3 mb. The invention contemplates the production of transgenic avians that carry either the light or the heavy chain or both the light and the heavy chain in their genome. For example, the loci may be present on one or more artificial chromosomes introduced into an avian's cells or may be introduced into the avian's genome by integrase mediated recombination as disclosed herein.

In one embodiment, two artificial chromosomes are produced, one containing the light chain and one containing the heavy chain. In one embodiment, each artificial chromosome may be used to produce a separate line of animal (e.g., two lines of chickens). The two lines are crossed and offspring are selected that carry heavy and light chain artificial chromosomes. In another embodiment, the two artificial chromosomes are co-introduced into the avian, e.g., co-injected into a germinal disc.

In another embodiment, an artificial chromosome may be created that carries both the heavy locus and light chain locus allowing generation of a single line of animals capable of producing antibodies.

In one embodiment of the invention, it is contemplated that the Ig gene(s) includes one or more additional variable region genes and/or one or more constant region genes which are not normally present in the Ig gene(s).

Ig genes are polymorphic, particularly in the variable coding regions. Therefore, Ig-artificial chromosomes can be produced that are capable of creating polyclonal antibodies that are specifically enhanced for a particular target antigen. For example, it is found that a human family is particularly resistant to the development of cancer, for example, a certain type of cancer such as breast cancer. The resistance trait is traced to their heavy and light chain genes, suggesting that this combination of heavy and light chain alleles can produce a mixture of antibodies that are exceptionally able to target and destroy cancer cells such as breast cancer cells. The heavy and light chain genes can be cloned from DNA extracted from a family member and inserted into an artificial chromosome. Therefore, in one embodiment of the invention, a transgenic animal such as a chicken carrying an artificial chromosome will produce polyclonal antibodies such that when immunized with cancer cells, or antigens thereof, such as breast cancer cells, or antigens thereof, polyclonal antibodies will be produced that can be used to treat cancer patients, for example, breast cancer patients.

The present invention provides for recombinant vertebrate cells (e.g., transgenic or transchromosomal avian cells) and transgenic vertebrate animals (e.g., transgenic or transchromosomal avians) and methods of making the cells and the animals. For example, the invention provides for methods of inserting nucleotide sequences into the genome of vertebrate animals or into the cells of vertebrate animals in a site specific manner. Examples of vertebrates include, without limitation, birds, mammals, fish, reptiles and amphibians. Examples of mammals include sheep, goats and cows. In one certain embodiment of the invention, the vertebrate animals are birds or avians. Examples of birds include, without limitation, chickens, turkeys, ducks, geese, quail, pheasants, parrots, finches, hawks, crows and ratites including ostriches, emu and cassowary. Methods disclosed herein for producing transgenic and transchromosomic avians are generally applicable for all avians. For example, though the size of the hard shell egg laid by avians may vary substantially (e.g., hummingbird eggs compared to ostrich eggs), the size and structure of the germinal disc is substantially the same among avians. Therefore, since the present invention, in large part, relies on the injection of large DNA molecules (e.g., artificial chromosomes) into a germinal disc, a practitioner in the art would expect that the invention will function universally among avians.

In one embodiment, the present invention provides for methods of inserting nucleotide sequences into the genome of an animal using methods of transgenesis based on site specific integration, for example, site specific integrase mediated-transgenesis. The present invention contemplates any useful method of integrase mediated transgenesis including but not limited to, transgenesis mediated by serine recombinases and tyrosine recombinases. Serine recombinases are well known in the art and include without limitation, EcoYBCK, ΦC31, SCH10.38c, SCC88.14, SC8F4.15c, SCD12A.23, Bxb1, WwK, Sau CcrB, Bsu CisB, TP901-1, Φ370.1, Φ105, ΦFC1, A118, Cac1956, Cac1951, Sau CcrA, Spn, TnpX, TndX, SPBc2, SC3C8.24, SC2E1.37, SCD78.04c, R4, ΦRv1, Y4bA, Bja, SsoISC1904b, SsoISC1904a, Aam, MjaMJ1004, Pab, SsoISC1913, HpyIS607, MceRv0921, MtuRv0921, MtuRv2979c, MtuRv2792c, MtuISY349, MtuRv3828c, SauSK1, Spy, EcoTn21, Mlo92, EcoTn3, Lla, Cpe, SauSK41, BmeTn5083, SfaTn917, Bme53, Ran, RmzY4CG, SarpNL1, Pje, Xan, ISXc5, Pae, Xca, Req, Mlo90, PpsTn5501, pMER05, Cgl, MuGin, StyHin, Xfa911, Xfa910, Rrh, SauTn552 and Aac serine recombinases. Tyrosine recombinases well known in the art include without limitation, BS codV, BS ripX, BS ydcL, CB tnpA, Col1D, CP4, Cre, D29, DLP12, DN int, EC FimB, EC FimE, EC orf, EC xerC, EC xerD, Φ11, Φ13, Φ80, Φadh, ΦCTX, ΦLC3, FLP, ΦR73, HIorf, HI rci, HI xerC, HI xerD, HK22, HP1, L2, L5, L54, X, LL orf, LL xerC, LO L5, MJ orf, ML orf, MP int, MT int, MT orf, MV4, P186, P2, P21, P22, P4, P434, PA sss, PM fimB, pAE1, pCL1, pKD1, pMEA, pSAM2, pSB2, pSB3, pSDL2, pSE101, pSE211, pSM1, pSR1, pWS58, R721, Rc1, SF6, SLP1, SM orf, SsrA, SSV1, T12, Tn21, Tn4430, Tn554a, Tn554b, Tn7, Tn916, Tuc, WZ int, XisA and XisC. Other enzymes which may be useful for mediation of transgenesis in accordance with the present invention include, certain transposases, invertases and resolvases.

In certain instances, integration host factors (IHF) may be necessary for the integration of nucleotide sequences of the invention into the genome of cells as disclosed herein. In such a case, the integration host factors may be delivered to the cells directly or they may be delivered to the cells in the form of a nucleic acid which, in the case of RNA, is translated to produce the IHF or, in the case of DNA, is transcribed and translated to produce the IHF.

The present invention contemplates the use of any system capable of site specifically inserting a nucleotide sequence of interest into the genome of a cell, for example, to produce a transgenic vertebrate animal. Typically, although not exclusively, these systems require at least three components: 1) a sequence in the genome which specifies the site of insertion; 2) a nucleotide sequence which is directed to the site of insertion and an enzyme which catalyzes the insertion of the nucleotide sequence into the genome at the site of insertion. Many enzymes, including integrases, which are capable of site specifically inserting nucleotide sequences into the genome have been characterized. Examples of these enzymes are disclosed in for example, Esposito et al (1997) Nucleic Acids Research, 25; 3605-3614 and Nunes-Düby et al (1998) Nucleic Acids Research, 26; 391-406. The disclosure of each of these references is incorporated herein in their entirety.

In one embodiment of the present invention, a serine recombinase is employed. Serine recombinase integrase mediates recombination between an attB site on a transgene vector and an attP or a pseudo attP site on a chromosome. In the method of the invention for integrase-mediated transgenesis, a heterologous wild-type attP site can be integrated into a nuclear genome to create a transgenic cell line or a transgenic vertebrate animal, such as an avian. A serine recombinase (integrase) and an attB-bearing transgene vector are then introduced into cells harboring the heterologous attP site, or into embryos derived from animals which bear the attP recombination site. The locations of attP and attB may be reversed such that the attB site is inserted into a chromosome and the attP sequence resides in an incoming transgene vector. In either case, the att site of the introduced vector would then preferentially recombine with the integrated heterologous att site in the genome of the recipient cell.

The methods of the invention are based, in part, on the discovery that there exists in vertebrate animal genomes, such as avian genomes, a number of specific nucleic acid sequences, termed pseudo-recombination sites, the sequences of which may be distinct from wild-type recombination sites but which can be recognized by a site-specific integrase and used to promote the efficient insertion of heterologous genes or polynucleotides into the targeted nuclear genome. The inventors have identified pseudo-recombination sites in avian cells capable of recombining with a recombination site, such as an attB site within a recombinant nucleic acid molecule introduced into the target avian cell. The invention is also based on the prior integration of a heterologous att recombination site, typically isolated from a bacteriophage or a modification thereof, into the genome of the target avian cell.

Integration into a predicted chromosomal site is useful to improve the predictability of expression, which is particularly advantageous when creating transgenic avians. Transgenesis by methods that result in insertion of the transgene into random positions of the avian genome is unpredictable since the transgene may not express at the expected levels or in the predicted tissues.

The invention as disclosed herein, therefore, provides methods for site-specifically genetically transforming an avian nuclear genome. In general, an avian cell having a first recombination site in the nuclear genome is transformed with a site-specific polynucleotide construct comprising a second recombination sequence and one or more polynucleotides of interest. Into the same cell, integrase activity may be introduced that specifically recognizes the first and second recombination sites under conditions such that the polynucleotide sequence of interest is inserted into the nuclear genome via an integrase-mediated recombination event between the first and second recombination sites.

The integrase activity, or a source thereof, can be introduced into the cell prior to, or concurrent with, the introduction of the site-specific construct. The integrase can be delivered to a cell as a polypeptide, or by expressing the integrase from a source polynucleotide such as an mRNA or from an expression vector that encodes the integrase, either of which can be delivered to the target cell before, during or after delivery of the polynucleotide of interest. Any integrase that has activity in a cell may be useful in the present invention, including HK022 (Kolot et al, (2003) Biotechnol. Bioeng. 84: 56-60). In one embodiment, the integrase is a serine recombinase as described, for example, by Smith & Thorpe, in Mol. Microbiol., 44: 299-307 (2002). For example, the integrase may be TP901-1 (Stoll et al, J. Bact., 184: 3657-3663 (2002); Olivares et al, Gene, 278:167-176 (2001) or the integrase from the phage phiC31.

The nucleotide sequence of the junctions between an integrated transgene into the attP (or attB site) would be known. Thus, a PCR assay can be designed by one of skill in the art to detect when the integration event has occurred. The PCR assay for integration into a heterologous wild-type attB or attP site can also be readily incorporated into a quantitative PCR assay using TAQMAN™ or related technology so that the efficiency of integration can be measured.

In one embodiment, the minimal attB and attP sites able to catalyze recombination mediated by the phiC31 integrase are 34 and 39 bp, respectively. In cell lines that harbor a heterologous integrated attP site, however, integrase may have a preference for the inserted attP over any pseudo-attP sites of similar length, because pseudo-attP sites have very low sequence identity (for example, between 10 to 50% identity) compared to the more efficient wild-type attP sequence. It is within the scope of the methods of the invention, however, for the recombination site within the target genome to be a pseudo-att site such as a pseudo-attP site or an attP introduced into a genome.

The sites used for recognition and recombination of phage and bacterial DNAs (the native host system) are generally non-identical, although they typically have a common core region of nucleic acids. In one embodiment, the bacterial sequence is called the attB sequence (bacterial attachment) and the phage sequence is called the attP sequence (phage attachment). Because they are different sequences, recombination can result in a stretch of nucleic acids (for example, attL or attR for left and right) that is neither an attB sequence or an attP sequence, and likely is functionally unrecognizable as a recombination site to the relevant enzyme, thus removing the possibility that the enzyme will catalyze a second recombination reaction that would reverse the first.

The integrase may recognize a recombination site where sequence of the 5′ region of the recombination site can differ from the sequence of the 3′ region of the recombination sequence. For example, for the phage phiC31 attP (the phage attachment site), the core region is 5′-TTG-3′ the flanking sequences on either side are represented here as attP5′ and attP3′, the structure of the attP recombination site is, accordingly, attP5′-TTG-attP3′. Correspondingly, for the native bacterial genomic target site (attB) the core region is 5′-TTG-3′, and the flanking sequences on either side are represented here as attB5′ and attB3′, the structure of the attB recombination site is, accordingly, attB5′-TTG-attB3′. After a single-site, phiC31 integrase-mediated recombination event takes place between the phiC31 phage and the bacterial genome, the result is the following recombination product: attB5′-TTG-attP3′{phiC31 vector sequences}-attP5′-TTG-attB3′. In the method of invention, the attB site will be within a recombinant nucleic acid molecule that may be delivered to a target cell. The corresponding attP (or pseudo-attP) site will be within the cell nuclear genome. Consequently, after phiC31 integrase mediated recombination, the recombination product, the nuclear genome with the integrated heterologous polynucleotide will have the sequence attP5′-TTG-attB3′{heterologous polynucleotide}-attB5′-TTG-attP3′. Typically, after recombination the post-recombination recombination sites are no longer able to act as substrate for the phiC31 integrase. This results in stable integration with little or no integrase mediated excision.

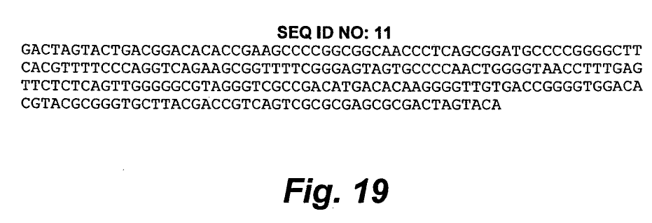

While the one useful recombination site to be included in the recombinant nucleic acid molecules and modified chromosomes of the present invention is the attP site, it is contemplated that any attP-like site may be used if compatible with the attB site. For instance, any pseudo-attP site of the chicken genome may be identified according to the methods of Example 7 herein and used as a heterologous att recombination site. For example, such attP-like sites may have a sequence that is greater than at least 25% identical to SEQ ID NO: 11 as shown in FIG. 19, such as described in Groth et al, Proc. Natl. Acad. Sci. U.S.A. 97: 5995-6000 (2000) incorporated herein by reference in its entirety. In one embodiment, the selected site will have a similar degree of efficiency of recombination, for example, at least the same degree of efficiency of recombination as the attP site (SEQ ID NO: 11) itself.

In the present invention, the recipient cell population may be an isolated cell line such as, for example, DF-1 chicken fibroblasts, chicken DT40 cells or a cell population derived from an early stage embryo, such as a chicken stage I embryo or mid stage or late stage (e.g., stage X) embryos. One useful avian cell population is blastodermal cells isolated from a stage X avian embryo. The methods of the present invention, therefore, include steps for the isolation of blastodermal cells that are then suspended in a cell culture medium or buffer for maintaining the cells in a viable state, and which allows the cell suspension to contact the nucleic acids of the present invention. It is also within the scope of the invention for the nucleic acid construct and the source of integrase activity to be delivered directly to an avian embryo such as a blastodermal layer, or to a tissue layer of an adult bird such as the lining of an oviduct.

When the recipient cell population is isolated from an early stage avian embryo, the embryos must first be isolated. For stage I avian embryos from, for example, a chicken, a fertilized ovum is surgically removed from a bird before the deposition of the outer hard shell has occurred. The nucleic acids for integrating a heterologous nucleic acid into a recipient cell genome may then be delivered to isolated embryos by lipofection, microinjection (as described in Example 6 below) or electroporation and the like. After delivery of the nucleic acid, the transfected embryo and its yolk may be deposited into the infundibulum of a recipient hen for the deposition of egg white proteins and a hard shell, and laying of the egg. Stage X avian embryos are obtained from freshly laid fertilized eggs and the blastodermal cells isolated as a suspension of cells in a medium, as described in Example 4 below. Isolated stage X blastodermal cell populations, once transfected, may be injected into recipient stage X embryos and the hard shell eggs resealed according to the methods described in U.S. Pat. No. 6,397,777, issued Jun. 4, 2002, the disclosure of which is incorporated in its entirety by reference herein.

In one embodiment of the invention, once a heterologous nucleic acid is delivered to the recipient cell, integrase activity is expressed. The expressed integrase (or injected integrase polypeptide) then mediates recombination between the att site of the heterologous nucleic acid molecule, and the att (or pseudo att) site within the genomic DNA of the recipient avian cell.

It is within the scope of the present invention for the integrase-encoding sequence and a promoter operably linked thereto to be included in the delivered nucleic acid molecule and that expression of the integrase activity occurs before integration of the heterologous nucleic acid into the cell genome. In one embodiment, an integrase-encoding nucleic acid sequence and associated promoter are in an expression vector that may be co-delivered to the recipient cell with the heterologous nucleic acid molecule to be integrated into the recipient genome.

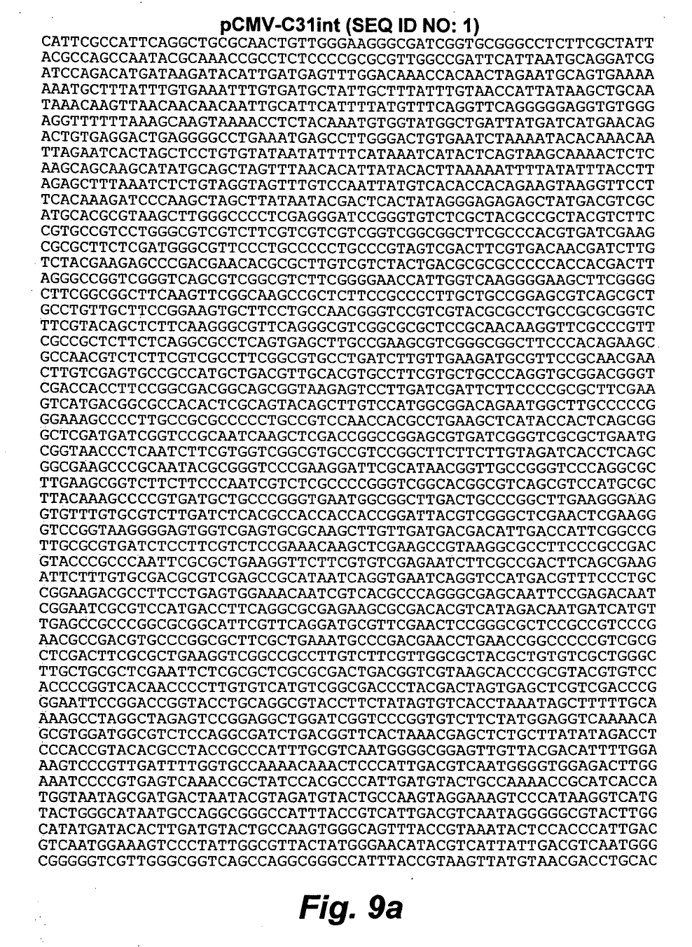

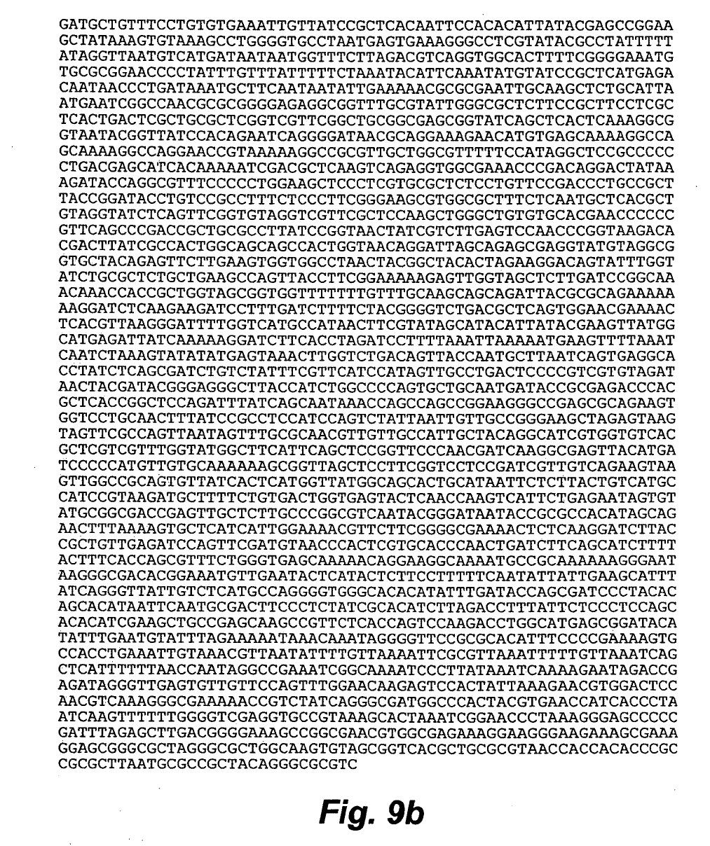

One suitable integrase expressing expression vector for use in the present invention is pCMV-C31int (SEQ ID NO: 1) as shown in FIG. 9, and described in Groth et al, Proc. Natl. Acad. Sci. U.S.A. 97: 5995-6000 (2000), incorporated herein by reference in its entirety. In pCMV-C31int, expression of the integrase-encoding sequence is driven by the CMV promoter. However, any promoter may be used that will give expression of the integrase in a recipient cell, including operably linked avian-specific gene expression control regions of the avian ovalbumin, lysozyme, ovomucin, ovomucoid gene loci, viral gene promoters, inducible promoters, the RSV promoter and the like.

The recombinant nucleic acid molecules of the present invention for delivery of a heterologous polynucleotide to the genome of a recipient cell may comprise a nucleotide sequence encoding the attB attachment site of Streptomyces ambofaciens as described in Thorpe & Smith, Proc. Natl. Acad. Sci. U.S.A. 95: 5505-5510 (1998). The nucleic acid molecule of the present invention may further comprise an expression cassette for the expression in a recipient cell of a heterologous nucleic acid encoding a desired heterologous polypeptide. Optionally, the nucleic acid molecules may also comprise a marker such as, but not limited to, a puromycin resistance gene, a luciferase gene, EGFP, and the like.

It is contemplated that the expression cassette, for introducing a desired heterologous polypeptide, comprises a promoter operably linked to a nucleic acid encoding the desired polypeptide and, optionally, a polyadenylation signal sequence. Exemplary nucleic acids suitable for use in the present invention are more fully described in the examples below.

In one embodiment of the present invention, following delivery of the nucleic acid molecule and a source of integrase activity into a cell population, for example, an avian cell population, the cells are maintained under culture conditions suitable for the expression of the integrase and/or for the integrase to mediate recombination between the recombination site of the nucleic acid and recombination site in the genome of a recipient cell. When the recipient cell is cultured in vitro, such cells may be incubated at 37° Celsius. For example, chicken early stage blastodermal cells may be incubated at 37° Celsius. They may then be injected into an embryo within a hard shell, which is resealed for incubation until hatching. Alternatively, the transfected cells may be maintained in in vitro culture.

In one embodiment, the present invention provides methods for the site-specific insertion of a heterologous nucleic acid molecule into the nuclear genome of a cell by delivering to a target cell that has a recombination site in its nuclear genome, a source of integrase activity, a site-specific construct that has another recombination site and a polynucleotide of interest, and allowing the integrase activity to facilitate a recombination event between the two recombination sites, thereby integrating the polynucleotide of interest into the nuclear genome.

(a) Expression vector nucleic acid molecules: A variety of recombinant nucleic acid expression vectors are suitable for use in the practice of the present invention. The site-specific constructs described herein can be constructed utilizing methodologies well known in the art of molecular biology (see, for example, Ausubel or Maniatis) in view of the teachings of the specification. As described above, the constructs are assembled by inserting into a suitable vector backbone a recombination site such as an attP or an attB site, a polynucleotide of interest operably linked to a gene expression control region of interest and, optionally a sequence encoding a positive selection marker. Polynucleotides of interest can include, but are not limited to, expression cassettes encoding a polypeptide to be expressed in the transformed cell or in a transgenic vertebrate animal derived therefrom. The site-specific constructs are typically, though not exclusively, circular and may also contain selectable markers, an origin of replication, and other elements.

Any of the vectors of the present invention may also optionally include a sequence encoding a signal peptide that directs secretion of the polypeptide expressed by the vector from the transgenic cells, for instance, from tubular gland cells of the oviduct of an avian. In one embodiment, this aspect of the invention effectively broadens the spectrum of exogenous proteins that may be deposited in the whites of avian eggs using the methods of the invention. Where an exogenous polypeptide would not otherwise be secreted, the vector bearing the coding sequence can be modified to comprise, for instance, about 60 bp encoding a signal peptide. The DNA sequence encoding the signal peptide may be inserted in the vector such that the signal peptide is located at the N-terminus of the polypeptide encoded by the vector.

The expression vectors of the present invention can comprise a transcriptional regulatory region, for example, an avian transcriptional regulatory region, for directing expression of either fusion or non-fusion proteins. With fusion vectors, a number of amino acids are usually added to the desired expressed target gene sequence such as, but not limited to, a polypeptide sequence for thioredoxin. A proteolytic cleavage site may further be introduced at a site between the target recombinant protein and the fusion sequence. Additionally, a region of amino acids such as a polymeric histidine region may be introduced to allow binding of the fusion protein to metallic ions such as nickel bonded to a solid support, for purification of the fusion protein. Once the fusion protein has been purified, the cleavage site allows the target recombinant protein to be separated from the fusion sequence. Enzymes suitable for use in cleaving the proteolytic cleavage site include, but are not limited to, Factor Xa and thrombin. Fusion expression vectors that may be useful in the present invention include pGex (Amrad Corp., Melbourne, Australia), pRIT5 (Pharmacia, Piscataway, N.J.) and pMAL (New England Biolabs, Beverly, Mass.), that fuse glutathione S-transferase, protein A, or maltose E binding protein, respectively, to a desired target recombinant protein.

Epitope tags are short peptide sequences that are recognized by epitope specific antibodies. A fusion protein comprising a recombinant protein and an epitope tag can be simply and easily purified using an antibody bound to a chromatography resin, for example. The presence of the epitope tag furthermore allows the recombinant protein to be detected in subsequent assays, such as Western blots, without having to produce an antibody specific for the recombinant protein itself. Examples of commonly used epitope tags include V5, glutathione-S-transferase (GST), hemaglutinin (HA), the peptide Phe-His-His-Thr-Thr, chitin binding domain, and the like.

Exemplary gene expression control regions for use in cells such as avian cells (e.g., chicken cells) include, but are not limited to, avian specific promoters such as the chicken lysozyme, ovalbumin, or ovomucoid promoters, and the like. Particularly useful in avian systems are tissue-specific promoters such as avian oviduct promoters that allow for expression and delivery of a heterologous polypeptide to an egg white.

Viral promoters serve the same function as bacterial or eukaryotic promoters and either provide a specific RNA polymerase in trans (bacteriophage T7) or recruit cellular factors and RNA polymerase (SV40, RSV, CMV). Viral promoters can be useful as they are generally particularly strong promoters. One useful promoter for employment in avian cells is the RSV promoter.

Selection markers are valuable elements in expression vectors as they provide a means to select for growth of only those cells that contain a vector. Common selectable marker genes include those for resistance to antibiotics such as ampicillin, puromycin, tetracycline, kanamycin, bleomycin, streptomycin, hygromycin, neomycin, ZEOCIN™, and the like.

Another element useful in an expression vector is an origin of replication. Replication origins are unique DNA segments that contain multiple short repeated sequences that are recognized by multimeric origin-binding proteins and that play a key role in assembling DNA replication enzymes at the origin site. Suitable origins of replication for use in expression vectors employed herein include E. coli oriC, colE1 plasmid origin, and the like.

A further useful element in an expression vector is a multiple cloning site or polylinker. Synthetic DNA encoding a series of restriction endonuclease recognition sites is inserted into a vector, for example, downstream of the promoter element. These sites are engineered for convenient cloning of DNA into the vector at a specific position.

Elements such as the foregoing can be combined to produce expression vectors suitable for use in the methods of the invention. Those of skill in the art will be able to select and combine the elements suitable for use in their particular system in view of the teachings of the present specification.

Provided for is the stable introduction of a large DNA molecule into the cell of an avian. In one particularly useful embodiment, the large DNA molecule is a chromosome. The chromosomes to be introduced into cells of an avian may be referred to herein as “artificial chromosomes”; however, the term “artificial chromosome” is not a limiting term and any useful large DNA molecule or chromosome may be employed in the present invention.

The present invention provides modified chromosomes, which are either isolated chromosomes or artificial chromosomes, which function as useful vectors to shuttle transgenes or gene clusters into the genome. By delivering the modified or artificial chromosome to an isolated recipient cell, the target cell, and progeny thereof, become trisomic or transchromosomic. Typically, an additional or triosomic chromosome will not affect the subsequent development of the recipient cell and/or an embryo, nor interfere with the reproductive capacity of an adult developed from such cells or embryos. The chromosome also should be stable within chicken cells. An effective method is also required to isolate a population of chromosomes for delivery into chicken embryos or early cells.

Chickens that are trisomic for microchromosome 16 have been described (Miller et al, Proc. Natl. Acad. Sci. U.S.A. 93: 3958-3962 (1996); Muscarella et al, J. Cell Biol. 101: 1749-1756 (1985). In these cases, triploidy and trisomy occurred naturally, and illustrate that an extra copy of one or more of the chicken chromosomes is compatible with normal development and reproductive capacity.

The transchromosomic avians resulting from the cellular introduction of an artificial chromosome typically will comprise cells which include the normal complement of chromosomes plus at least one additional chromosome. In one embodiment, about 0.001% to 100% of the cells of the avian will include an additional chromosome. In another embodiment, about 0.1% to 100% of the cells of the avian will include an additional chromosome. In another embodiment, about 5% to 100% of the cells of the avian will include an additional chromosome. In another embodiment, about 10% to 100% of the cells of the avian will include an additional chromosome. In another embodiment, about 50% to 100% of the cells of the avian will include an additional chromosome. In one particularly useful embodiment, the additional chromosome is transmitted through the germ-line of the transchromosomic avian and many, for example, most (i.e., more than 50%) of the cells of the offspring avians will include the additional chromosome. The invention contemplates the introduction and propagation of any useful number of chromosomes into the cell(s) of a transgenic avian or isolated avian cells. For example, the invention contemplates one artificial chromosome or two artificial chromosomes or three artificial chromosomes stably incorporated into the genome of the cell(s) of a transchromosomal avian or isolated avian cells.

Any or all tissues of the transchromosomic avian can include the artificial chromosome. In one useful embodiment, one or more cells of the oviduct of the avians include the additional chromosome. For example, tubular gland cells of the oviduct may include the additional chromosome.

A number of artificial chromosomes are useful in the methods of the invention, including, for instance, a human chromosome modified to work as an artificial chromosome in a heterologous species as described, for example, for mice (Tomizuka et al, Proc. Natl. Acad. Sci. U.S.A. 97: 722-727 (2000); for cattle (Kuroiwa et al, Nat. Biotechnol. 20: 889-894 (2002); a mammalian artificial chromosome used in mice (Co et al, Chromosome Res. 8: 183-191 (2000).

Examples of large nucleic acid molecules include, but are not limited to, natural chromosomes and fragments thereof, for example, chromosomes (e.g., mammalian chromosomes) and fragments thereof which retain a centromere, artificial chromosome expression systems (satellite DNA-based artificial chromosomes (SATACs); see U.S. Pat. Nos. 6,025,155, issued Feb. 15, 2000 and 6,077,697 issued Jun. 20, 2000, the disclosures of which are incorporated herein in their entirety by reference), mammalian artificial chromosomes (MACs) (e.g., HACs), plant artificial chromosomes, insect artificial chromosomes, avian artificial chromosomes and minichromosomes (see, e.g., U.S. Pat. Nos. 5,712,134 issued Jan. 27, 1998; 5,891,691, issued Apr. 6, 1999; 5,288,625, issued Feb. 22, 1994; 6,743,967 issued Jun. 1, 2004; and U.S. patent application Ser. Nos. 10/235,119, published Jun. 19, 2003, the disclosure of each of these six patents and the patent application are incorporated herein in their entirety by reference). Also contemplated for use herein are YACs, BACs, bacteriophage-derived artificial chromosomes (BBPACs), cosmid or P1 derived artificial chromosomes (PACs).

As used herein, a large nucleic acid molecule such as artificial chromosomes can stably replicate and segregate alongside endogenous chromosomes in a cell. It has the capacity to act as a gene delivery vehicle by accommodating and expressing foreign genes contained therein. A mammalian artificial chromosome (MAC) refers to chromosomes that have an active mammalian centromere(s). Plant artificial chromosomes, insect artificial chromosomes and avian artificial chromosomes refer to chromosomes that include plant, insect and avian centromeres, respectively. A human artificial chromosome (HAC,) refers to chromosomes that include human centromeres. For exemplary artificial chromosomes, see, e.g., U.S. Pat. Nos. 6,025,155, issued Feb. 15, 2000; 6,077,697, issued Jun. 20, 2000; 5,288,625, issued Feb. 22, 1994; 5,712,134, issued Jan. 27, 1998; 5,695,967, issued Dec. 9, 1997; 5,869,294, issued Feb. 9, 1999; 5,891,691, issued Apr. 6, 1999 and 5,721,118, issued Feb. 24, 1998 and published International PCT application Nos., WO 97/40183, published Oct. 30, 1997 and WO 98/08964, published Mar. 5, 1998, the disclosure of each of these eight patents and two PCT applications are incorporated in their entirety herein by reference.

The large nucleic acid molecules (e.g., chromosomes) can include a single copy of a desired nucleic acid fragment encoding a particular nucleotide sequence, such as a gene of interest (e.g., transgene of interest), or can carry multiple copies thereof or multiple genes, different heterologous nucleotide sequences or expression cassettes or may encode one or more heterologous transcripts each encoding more than one useful protein product (for example, the transcript(s) may comprise an IRES). Any useful IRES may be employed in the invention. See, for example, U.S. Pat. No. 4,937,190, issued Jan. 26, 1990; Nature (1988) 334:320-325; J Virol (1988) 62:3068-3072; Cell (1992) 68:119-131; J Virol (1990) 64; 4625-4631; and J Virol (1992) 66:1476-1483, the disclosures of which are incorporated in their entirety herein by reference, which disclose useful IRESs. For example, the nucleic acid molecules can carry 40 or even more copies of genes of interest. The large nucleic acid molecules can be associated with proteins, for example, chromosomal proteins, that typically function to regulate gene expression and/or participate in determining overall structure (e.g., nucleosomes).

Certain useful artificial chromosomes, such as satellite DNA-based artificial chromosomes, can include substantially all neutral non-coding sequences (heterochromatin) except for foreign heterologous, typically gene-encoding, nucleic acid (see U.S. Pat. Nos. 6,025,155, issued Feb. 15, 2000 and 6,077,697, issued Jun. 20, 2000 and International PCT application No. WO 97/40183, published Oct. 30, 1997 and Lindenbaum et al Nucleic Acids Res (2004) vol 32 no. 21 e172, the disclosures of these two patents, the PCT application and the publication are incorporated in their entirety herein by reference). Foreign genes (i.e., nucleotide sequences of interest) contained in these artificial chromosomes can include, but are not limited to, nucleic acid that encodes therapeutically effective substances (e.g., therapeutic proteins such as those disclosed elsewhere herein and traceable marker proteins (reporter genes), such as fluorescent proteins, such as green, blue or red fluorescent proteins (GFP, BFP and RFP, respectively), other reporter genes, such as beta-galactosidase and proteins that confer drug resistance, such as a gene encoding hygromycin-resistance.

Preferably, the artificial chromosomes employed herein do not interfere with the host cells' processes and can be easily purified by useful purification methods such as large-scale by high-speed flow cytometry. See, for example, de Jong, G, et al. Cytometry 35: 129-33, 1999, the disclosure of which is incorporated herein in its entirety by reference. In one embodiment, flow cytometry is employed to purify chromosomes according to de Jong supra, with the exception that the Hoechst 33258 used to stain the chromosome suspension prior to flow cytometric sorting is diluted to a concentration of about 0.125 μg/ml opposed to 2.5 μg/ml. Such artificial chromosomes are useful for the production of transchromosomic chickens produced by introduction of the chromosomes into certain cells, for example, the germline cells, of an avian. In one particularly useful embodiment of the present invention, the transchromosomic chickens are produced by microinjection of the chromosomes, for example, cytoplasmic injection of the chromosomes into avian embryos, for example, early stage embryos such as a Stage I embryos, see, for example, U.S. patent application Ser. No. 10/679,034, filed Oct. 2, 2003, the disclosure of which is incorporated in its entirety herein by reference.

In one embodiment, heterologous nucleic acid is introduced into an artificial chromosome. Any useful method to introduce the nucleic acid into the chromosome may be employed in the invention. Thereafter, the artificial chromosomes are isolated in a mixture substantially free of other chromosomes or cellular material. For example, artificial chromosomes may be isolated by flow cytometry (e.g., dual laser high-speed flow cytometer as described previously (de Jong, G, et al. Cytometry 35: 129-33, 1999). See, for example, US Patent Application Publication No. 20030113917, published Jun. 19, 2003, the disclosure of which is incorporated in its entirety herein by reference.

In accordance with the present invention, any useful number of artificial chromosomes may be introduced into an avian cell (e.g., injected), for example, an avian germinal cell such as a cell of an ova, an embryo or a germinal disc of an avian egg. Any useful method of introducing the chromosomes into the avian cell is contemplated for use in the present invention. In addition, the invention contemplates the introduction of any useful number of chromosomes into an avian cell. For example, and without limitation, the invention contemplates the introduction of 1 to about 1,000,000 chromosomes injected per egg. In one embodiment, 1 to about 100,000 chromosomes are injected per egg. In another embodiment about 5 to about 100,000 artificial chromosomes are injected per egg. For example, about 10 to about 50,000 chromosomes may be injected per egg.

In one embodiment, there is a lower hatch rate for eggs injected with more than a certain number of chromosomes. In one embodiment, an injection of over 100,000 chromosomes reduces or brings the hatch rate to zero. In another embodiment, an injection of over 20,000 chromosomes reduces or brings the hatch rate to zero. In another embodiment, an injection of over 5,000 chromosomes reduces or brings the hatch rate to zero. In another embodiment, an injection of over 2,000 chromosomes reduces or brings the hatch rate to zero. For example, an injection of over 1,000 (e.g., 550) chromosomes reduces or brings the hatch rate to zero.

For injection, any useful volume of injection buffer may be used for each injection. For example, about 1 nl to about 1 μl may be injected. In addition, any useful concentration of chromosomes may be employed in the injection buffer. For example, and without limitation, 1 to about 100,000 chromosomes per microliter may be used. In addition, any useful number of injections may be performed on each egg.

In one embodiment, a concentration of 7000-11,500 chromosomes is used per μl of injection buffer (Monteith, D, et al. Methods Mol Biol 240: 227-242, 2004). In one embodiment, 25-100 nanoliters (nl) of injection buffer is used per injection.

Any useful avian embryos may be employed in the present invention. For example, the embryos may be collected from 24-36 week-old hens (e.g., commercial White Leghorn variety of G. gallus). In one embodiment, a germinal disc is injected with the chromosomes. In one embodiment, the embryo donor hens are inseminated weekly using pooled semen from roosters to produce eggs for injection. Any useful method, such as methods known to those skilled in the art, may be employed to collect fertilized eggs.

Cytoplasmic injection of artificial chromosomes can be achieved by employing certain microinjection systems or assemblies. In one particularly useful embodiment, the microinjection assembly or microinjection system disclosed in U.S. patent application Ser. No. 09/919,143, filed Jul. 31, 2001 (the '143 application), now abandoned, the disclosure of which is incorporated herein in its entirety, is employed. Use of such a cytoplasmic injection device allows for the precise delivery of chromosomes into the cytoplasm of avian embryos, for example, early stage avian embryos, e.g., Stage I embryos.

Typically, following microinjection, the embryos are transferred to the oviduct of recipient hens utilizing any useful technique, such as that disclosed in Olsen, M and Neher, B. (1948) J Exp Zool 109: 355-66 followed by incubation and hatching of the birds.

Any useful method, such as PCR, may be used to test for the production of transchromosomic avians. Typically, the identification of a transchromosomic offspring is confirmed by fluorescence in-situ hybridization (FISH) and/or DNA analysis such as Southern blot or the like. In one useful embodiment, artificial chromosomes can be used as vectors to introduce large DNA payloads, such as nucleotide sequences to be expressed heterologously in the avian to yield a desired biomolecule, of stably maintained genetic information into transgenic chickens. Production of germline transchromosomic avians is confirmed by the production of transchromosomic offspring from the G0 birds.

The present invention provides for the introduction of desired nucleotide sequences into a chromosome, the chromosome of which can subsequently be isolated/purified and thereafter introduced into an avian as disclosed herein.

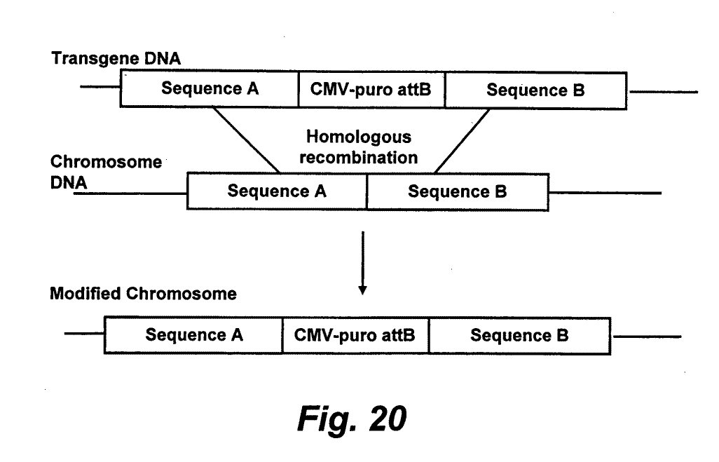

A useful chromosome isolation protocol can comprise the steps of inserting a lac-operator sequence (Robinett et al J. Cell Biol. 135: 1685-1700 (1996) into an isolated chromosome and, optionally, inserting a desired transgene sequence within the same chromosome. In one embodiment, the lac operator region is a concatamer of a plurality of lac operators for the binding of multiple lac repressor molecules. Insertion can be accomplished, for instance, by identifying a region of known nucleotide sequence associated with a particular avian chromosome. A recombinant DNA molecule may be constructed that comprises the identified region, a recombination site such as attB or attP and a lac-operator concatamer. The recombinant molecule is delivered to an isolated avian cell, for example, but not limited to, chicken DT40 cells that have elevated homologous recombination activity compared to other avian cell lines, whereupon homologous recombination will integrate the heterologous recombination site and the lac-operator concatamer into the targeted chromosome as shown in the schema illustrated in FIG. 20. A tag-polypeptide comprising a label domain and a lac repressor domain is also delivered to the cell, for example, by expression from a suitable expression vector. The nucleotide sequence coding for a GFP-lac-repressor fusion protein (Robinett et al, J. Cell Biol. 135: 1685-1700 (1996)) may be inserted into the same chromosome as the lac-operator insert. The lac repressor sequence, however, can also be within a different chromosome. An inducible promoter may also be used to allow the expression of the GFP-lac-repressor only after chromosome is to be isolated.

Induced expression of the GPF-lac-repressor fusion protein will result in specific binding of the tag fusion polypeptide to the lac-operator sequence for identification and isolation of the genetically modified chromosome. The tagged mitotic chromosome can be isolated using, for instance, flow cytometry as described in de Jong et al Cytometry 35: 129-133 (1999) and Griffin et al Cytogenet. Cell Genet. 87: 278-281 (1999).

A tagged chromosome can also be isolated using microcell technology requiring treatment of cells with the mitotic inhibitor colcemid to induce the formation of micronuclei containing intact isolated chromosomes within the cell. Final separation of the micronuclei is then accomplished by centrifugation in cytochalasin as described by Killary & Fournier in Methods Enzymol. 254: 133-152 (1995). Further purification of microcells containing only the desired tagged chromosome could be done by flow cytometry. It is contemplated, however, that alternative methods to isolate the mitotic chromosomes or microcells, including mechanical isolation or the use of laser scissors and tweezers, and the like.

The present invention envisions the employment of any useful protein-DNA binding or interaction to assist in isolating/purifying chromosomes of the invention. Such other methods in which a desired chromosome can be labeled for purposes of isolation/purification, are well known in the art including but not limited to, steroid receptor (such as the glucocorticoid receptor):site specific response element systems, see, for example, McNally et al (2000) Science 287:1262-1265; the bacteriophage lambda repressor system; and human homeobox genes. In addition, certain mutant forms of proteins which are employed in these systems (e.g., mutant proteins which bind there substrate with greater affinity than the non-mutant form of the protein) can be particularly useful for chromosome tagging (i.e., association of the chromosome with a marker that allows distinction of the chromosome, for example, distinction from cellular components such as other chromosomes) from other and subsequent isolation/purification of the chromosomes. Furthermore the invention contemplates the use of one or more selectable markers to identify cells which contain chromosomes comprising an introduced sequence of interest.

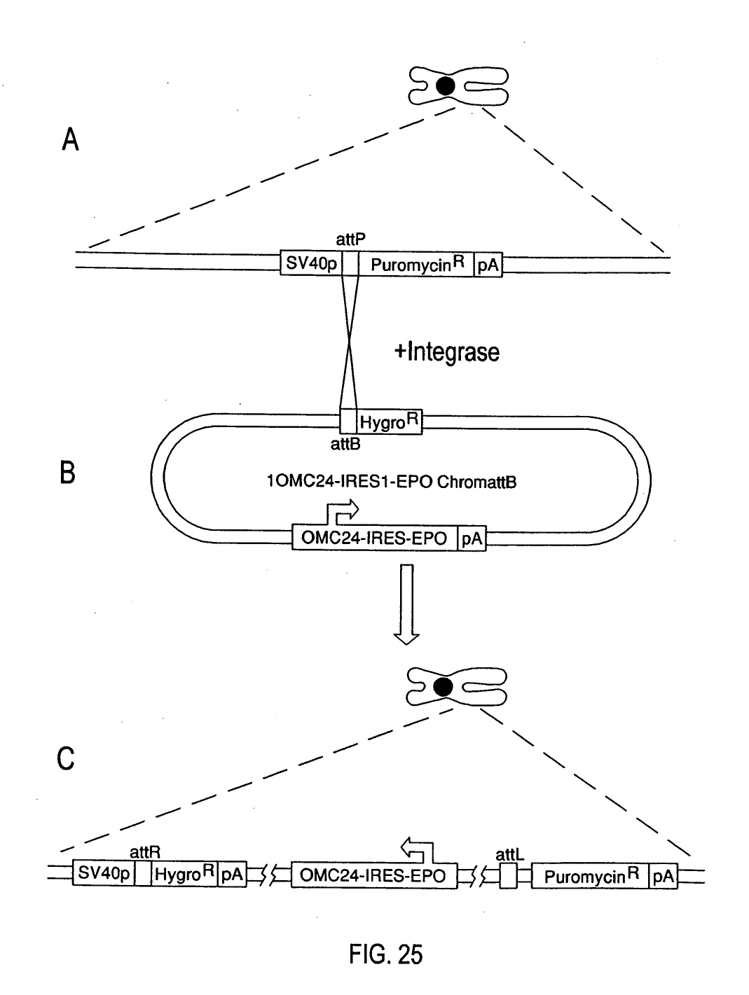

One specific embodiment of the making of a recombinant artificial chromosome can be seen in FIG. 25. In this embodiment the artificial chromosome includes a promoter which expresses a marker. Shown in FIG. 25 is an SV40 promoter, however, any useful promoter may be employed. For example, any promoter which will facilitate transcription in a cell line in which the artificial chromosome is present may be employed. For example, and without limitation, the following promoters may be useful: Pol III promoters (including type 1, type 2 and type 3 Pol III promoters) such as H1 promoters, U6 promoters, tRNA promoters, RNase MPR promoters and functional portions of each of these promoters. Other promoters that may be useful include, without limitation, Pol I promoters, Pol II promoters, cytomegalovirus (CMV) promoters, rous-sarcoma virus (RSV) promoters, murine leukemia virus (MLV) promoters, mouse mammary tumor virus (MMTV) promoters, ovalbumin promoters, lysozyme promoters, conalbumin promoters, ovomucoid promoters, ovomucin promoters, ovotransferrin promoters and functional portions of each of these promoters.

The schematic of FIG. 25 shows a vector which includes an OMC24-IRES-EPO nucleotide sequence of interest and a marker coding sequence both contained on a vector which integrates into the artificial chromosome. FIG. 25 shows a hygromycin resistance marker being employed. However, any useful marker (e.g., antibiotic resistant marker) may be used. For example, and without limitation, zeomycin resistance, neomycin resistance and blastomycin resistance markers can be used. Also shown is the use of an attP present on the artificial chromosome and an attB site present on the vector. However, any useful recombination sites and integrase may be employed such as those disclosed elsewhere herein.

In one embodiment, a useful cell line such as LMTK-containing the chromosome (A) in FIG. 25 is transfected with the vector B by standard methodologies such as lipofection. After introduction of the vector (B) into the artificial chromosome containing cell line, integration occurs, for example, between integration sites such as lambda attB and attP sites, wherein the hygromycin marker is expressed in the cells which contain the recombined artificial chromosome allowing for selection of the cells. For the employment of such integration sites, integrase or an integrase encoding gene is typically also introduced into the cell. In one useful embodiment, a lambda integrase gene is used which produces an integrase protein with a substitution mutation at the glutamine residue at position 174 to a lysine. This mutation removes the requirement for host factors allowing the integrase to function in cell lines. A practitioner of skill in the art will recognize that many variations to this basic recombination methodology may be employed.

It is contemplated that more than one, for example, between 1 and 100 rounds of integration of a nucleotide sequence of interest into the artificial chromosome may be performed. For example, one, two, three, four or more rounds of integration may be performed. In certain useful instances of multiple insertions of nucleotide sequences of interest into the artificial chromosome, it can be advantageous to employ different selectable markers. Any useful selectable markers can be employed in the case of multiple insertions, for example, and without limitation, genes which provide for resistance to hygromycin, zeomycin, neomycin and blastomycin can be used.

In one embodiment, multiple integration sites (e.g., multiple attP sites) are present in the artificial chromosome. Multiple rounds of integration can be performed to obtain insertions of more than one nucleotide sequence of interest in an artificial chromosome or to obtain an artificial chromosome with multiple copies of the same nucleotide sequence of interest. After each round of integration, a different marker can be used for each round of integration. For example, the nucleotide sequence to be inserted can include a hygromycin resistance coding sequence in the first round of integration, in the second round of integration the nucleotide to be inserted can include a zeomycin resistance marker coding sequence, in the third round of integration the nucleotide sequence to be inserted can include a neomycin resistance marker coding sequence and, in the fourth round of integration the nucleotide sequence to be inserted can include a blastomycin resistance marker coding sequence. A round of integration is where a nucleotide sequence of interest is introduced into the cell containing the artificial chromosome in which integration of the nucleotide sequence takes place as disclosed herein (e.g., integration into a site having a promoter proximal to the integration site which is operable to express the marker). This is merely an example of a method for integration of multiple nucleotide sequences of interest into an artificial chromosome for use as disclosed herein. The employment of other useful methodologies for integration of multiple nucleotide sequences into an artificial chromosome, as are understood by a practitioner of skill in the art, is included within the scope of the invention.

In certain instances it can be useful to rescue an artificial chromosome from an avian or cultured avian cell which contains the artificial chromosome in its genome. For example, certain artificial chromosomes may fragment after being introduced into an avian. During fragmentation, the artificial chromosome can be reduced substantially in size. (e.g., reduced by about 50% or about 60% or about 70% or about 80% or about 90% or more in size). However, after fragmentation the artificial chromosome can stabilize and as such is no longer susceptible to significant fragmentation or degradation in avian cells. These stabilized artificial chromosomes can be particularly useful for efficiently producing transchromosomic avians because, since the chromosomes are stabilized, they do not further fragment upon introduction into an avian or avian cell.

After recovery of the artificial chromosome from cells of the transchromosomic avian in which the artificial chromosome was originally introduced, the chromosome can be re-introduced into a cell line such as a CHO cell line in which a nucleotide sequence of interest is introduced into an integration site present in the artificial chromosome, as disclosed herein, producing a recombinant stabilized artificial chromosome. In one embodiment, multiple integration sites of the same type are present on the artificial chromosome thereby increasing the chance that one or more of the integration sites remain present on the artificial chromosome after the fragmentation occurs. The recombinant stabilized chromosome can then be introduced into an avian embryo to produce a line of avians containing in their genome a stabilized artificial chromosome comprising a transgene.

Any useful method may be used to recover the stabilized artificial chromosomes from the bird. For example, intact chromosomes can be prepared from blood cells of the birds as is known in the art, for example, essentially as disclosed in de Jong et al. Cytometry 35: 129-133 (1999) and Griffin et al. Cytogenet. Cell Genet. 87: 278-281 (1999). After preparation, the chromosomes are flow sorted to isolate the stabilized chromosome. In one embodiment, the stabilized artificial chromosome is of a size that does not allow it to be easily distinguished from the debris field in the cytometry histogram. In this instance sequence specific marker dyes can be used to tag the stabilized artificial chromosome thereby facilitating its purification. In one useful embodiment, polyamide probes can be used to tag the stabilized artificial chromosome as disclosed herein.

Typically, the artificial chromosomes introduced into avians are stably maintained in the avians and are passed to offspring through the germline. In addition, artificial chromosomes can be stably maintained in avian cell lines such as chicken cell line (DT-40).

The invention is also useful for visualizing gene activity in avian cells as is understood by a practitioner of ordinary skill in the art (See, for example, Tsukamoto, et al (2000) Nature Cell Biology, 2:871-878).

Most non-viral methods of gene transfer rely on normal mechanisms used by eukaryotic cells for the uptake and intracellular transport of macromolecules. In certain useful embodiments, non-viral gene delivery systems of the present invention rely on endocytic pathways for the uptake of the subject transcriptional regulatory region and operably linked polypeptide-encoding nucleic acid by the targeted cell. Exemplary gene delivery systems of this type include liposomal derived systems, poly-lysine conjugates, and artificial viral envelopes. Modified chromosomes as described above may be delivered to isolated avian embryonic cells for subsequent introduction to an embryo.

In a representative embodiment, a nucleic acid molecule can be entrapped in liposomes bearing positive charges on their surface (e.g., lipofectins) and (optionally) which are tagged with antibodies against cell surface antigens of the target tissue (Mizuno et al, 1992, NO Shinkei Geka 20: 547-551; PCT publication WO91/06309, published May 16, 1991; Japanese patent application 1047381, published Feb. 21, 1989; and European patent publication EP-A-43075, published Jan. 6, 1982, all of which are incorporated herein by reference in their entireties).

In similar fashion, the gene delivery system can comprise an antibody or cell surface ligand that is cross-linked with a gene binding agent such as polylysine (see, for example, PCT publications WO93/04701, published Mar. 18, 1993; WO92/22635, published Dec. 23, 1992; WO92/20316, published Nov. 26, 1992; WO92/19749, published Nov. 12, 1992; and WO92/06180, published Apr. 16, 1992, the disclosures of which are incorporated herein by reference in their entireties). It will also be appreciated that effective delivery of the subject nucleic acid constructs via receptor-mediated endocytosis can be improved using agents which enhance escape of genes from the endosomal structures. For instance, whole adenovirus or fusogenic peptides of the influenza HA gene product can be used as part of the delivery system to induce efficient disruption of DNA-containing endosomes (Mulligan et al, 1993, Science 260:926-932; Wagner et al, 1992, Proc. Natl. Acad. Sci. 89:7934-7938; and Christiano et al, 1993, Proc. Natl. Acad. Sci. 90:2122-2126, all of which are incorporated herein by reference in their entireties). It is further contemplated that a recombinant nucleic acid molecule of the present invention may be delivered to a target host cell by other non-viral methods including by gene gun, microinjection, sperm-mediated transfer, or the like.

In one embodiment of the invention, an expression vector that comprises a recombination site, such as an attB site, and a region encoding a polypeptide deposited into an egg white are delivered to oviduct cells by in vivo electroporation. In this method, the luminal surface of an avian oviduct is surgically exposed. A buffered solution of the expression vector and a source of integrase activity such as a second expression vector expressing integrase (for example, pCMV-int) is deposited on the luminal surface. Electroporation electrodes are then positioned on either side of the oviduct wall, the luminal electrode contacting the expression vector solution. After electroporation, the surgical incisions are closed. The electroporation will deliver the expression vectors to some, if not all, treated recipient oviduct cells to create a tissue-specific chimeric animal. Expression of the integrase allows for the integration of the heterologous polynucleotide into the genome of recipient oviduct cells. While this method may be used with any bird, a useful recipient is a chicken due to the size of the oviduct. Also useful is a transgenic bird that has a transgenic attP recombinant site in the nuclear genomes of recipient oviduct cells, thus increasing the efficiency of integration of the expression vector.

The attB/P integrase system is useful in the in vivo electroporation method to allow the formation of stable genetically transformed oviduct cells that otherwise progressively lose the heterologous expression vector.

The stably modified oviduct cells will express the heterologous polynucleotide and deposit the resulting polypeptide into the egg white of a laid egg. For this purpose, the expression vector will further comprise an oviduct-specific promoter such as ovalbumin or ovomucoid operably linked to the desired heterologous polynucleotide.

Another aspect of the invention is the generation of a trisomic or transchromosomic avian cell comprising a genetically modified extra chromosome. The extra chromosome may be an artificial chromosome or an isolated avian chromosome that has been genetically modified. Introduction of the extra chromosome to an avian cell will generate a trisomic or transchromosomic cell with 2n+1 chromosomes, where n is the haploid number of chromosomes of a normal avian cell.

Delivery of an isolated chromosome into an isolated avian cell or embryo can be accomplished in several ways. Isolated mitotic chromosomes or a micronucleus containing an interphase chromosome can be injected into early stage I embryos by cytoplasmic injection. The injected zygote would then be surgically transferred to a recipient hen for the production and laying of a hard shell egg. This hard shell egg would then be incubated until hatching of a chick.

In one embodiment, isolated microcells which contain the artificial chromosome can be fused to primordial germ cells (PGCs) isolated from the blood stream of late stage 15 embryos as described by Killary & Fournier in Methods Enzymol. 254: 133-152 (1995). The PGC/microcell hybrids can then be transplanted into the blood stream of a recipient embryo to produce germline chimeric chickens. (See Naito et al (1994) Mol. Reprod. Dev. 39: 153-161). The manipulated eggs would then be incubated until hatching of the bird.

Blastodermal cells isolated from stage X embryos can be transfected with isolated mitotic chromosomes. Following in vitro transfection, the cells are transplanted back into stage X embryos as described, for example, in Etches et al, Poult. Sci., 72: 882-889 (1993), and the manipulated eggs are incubated to hatching.

Stage X blastodermal cells can also be fused with isolated microcells and then transplanted back into to stage X embryos or fused to somatic cells to be used as nuclear donors for nuclear transfer as described by Kuroiwa et al, Nat. Biotechnol. 20: 889-894 (2002).

Chromosomal vectors, as described above, may be delivered to a recipient avian cell by, for example, microinjection, liposomal delivery or microcell fusion.

In the methods of the invention, a site-specific integrase is introduced into an avian cell whose genome is to be modified. Methods of introducing functional proteins into cells are well known in the art. Introduction of purified integrase protein can ensure a transient presence of the protein and its activity. Thus, the lack of permanence associated with most expression vectors is not expected to be detrimental.

The integrase used in the practice of the present invention can be introduced into a target cell before, concurrently with, or after the introduction of a site-specific vector. The integrase can be directly introduced into a cell as a protein, for example, by using liposomes, coated particles, or microinjection, or into the blastodermal layer of an early stage avian embryo by microinjection. A source of the integrase can also be delivered to an avian cell by introducing to the cell an mRNA encoding the integrase and which can be expressed in the recipient cell as an integrase polypeptide. Alternately, a DNA molecule encoding the integrase can be introduced into the cell using a suitable expression vector.

The present invention provides novel nucleic acid vectors and methods of use that allow integrases, such as phiC31 integrase, to efficiently integrate a heterologous nucleic acid into a vertebrate animal genome, for example, an avian genome. A novel finding is that the phiC31 integrase is remarkably efficient in avian cells and increases the rate of integration of heterologous nucleic acid at least 30-fold over that of random integration. Furthermore, the phiC31 integrase works equally well at 37° C. and 41° C., indicating that it will function in the environment of the developing avian embryo, as shown in Example 1.

It is important to note that the present invention is not bound by any mechanism or theory of operation. For example, the mechanism by which integrase, or any other substance described herein, facilitates transgenesis is unimportant. Integrase, for example, may facilitate transgenesis by mediating the integration of DNA into the genome of a recipient cell or integrase may facilitate transgenesis by facilitating the entry of the DNA into the cell or integrase may facilitate transgenesis by some other mechanism.

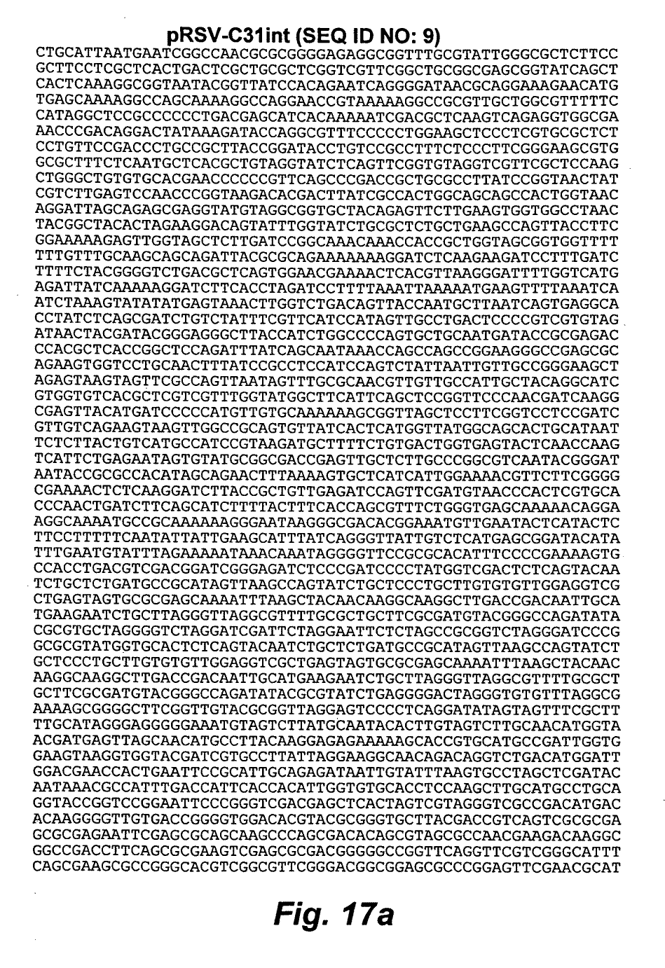

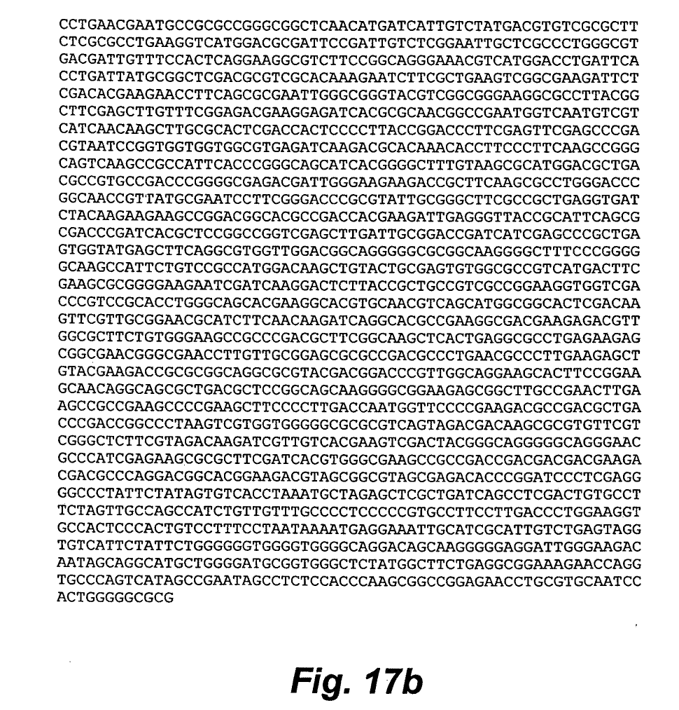

The site-specific vector components described above are useful in the construction of expression cassettes containing sequences encoding an integrase. One integrase-expressing vector useful in the methods of the invention is pCMV-C31int (SEQ ID NO: 1 as shown in FIG. 9) where the phiC31 integrase is encoded by a region under the expression control of the strong CMV promoter. Another useful promoter is the RSV promoter as used in SEQ ID NO: 9 shown in FIG. 17. Expression of the integrase is typically desired to be transient. Accordingly, vectors providing transient expression of the integrase are useful. However, expression of the integrase can be regulated in other ways, for example, by placing the expression of the integrase under the control of a regulatable promoter (i.e., a promoter whose expression can be selectively induced or repressed).

Delivery of the nucleic acids introduced into cells, for example, embryonic cells (e.g., avian cells), using methods of the invention may also be enhanced by mixing the nucleic acid to be introduced with a nuclear localization signal (NLS) peptide prior to introduction, for example, microinjection, of the nucleic acid. Nuclear localization signal (NLS) sequences are a class of short amino acid sequences which may be exploited for cellular import of linked cargo into a nucleus. The present invention envisions the use of any useful NLS peptide, including but not limited to, the NLS peptide of SV40 virus T-antigen.

An NLS of the invention is an amino acid sequence which mediates nuclear transport into the nucleus, wherein deletion of the NLS reduces transport into the nucleus. In certain embodiments, an NLS is a cationic peptide, for example, a highly cationic peptide. The present invention includes the use of any NLS sequence, including but not limited to, SV40 virus T-antigen. NLSs known in the art include, but are not limited to those discussed in Cokol et al, 2000, EMBO Reports, 1(5):411-415, Boulikas, T., 1993, Crit. Rev. Eukaryot. Gene Expr., 3:193-227, Collas, P. et al, 1996, Transgenic Research, 5: 451-458, Collas and Alestrom, 1997, Biochem. Cell Biol. 75: 633-640, Collas and Alestrom, 1998, Transgenic Research, 7: 303-309, Collas and Alestrom, Mol. Reprod. Devel., 1996, 45:431-438. The disclosure of each of these references is incorporated by reference herein in its entirety.

Not to be bound by any mechanism of operation, DNA is protected and hence stabilized by cationic polymers. The stability of DNA molecules in the cytoplasm of cells may be increased by mixing the DNA to be introduced, for example, microinjected with cationic polymers (for example, branched cationic polymers), such as polyethylenimine (PEI), polylysine, DEAE-dextran, starburst dendrimers, starburst polyamidoamine dendrimers, and other materials that package and condense the DNA molecules (Kukowska-Latallo et al, 1996, Proc. Natl. Acad. Sci. USA 93:4897-4902).

Once the DNA molecules are delivered to the cytoplasm of cells, they migrate into the cell's endocytotic vesicles. Furthermore, migration into the cell's endosome is followed by fast inactivation of DNA within the endolysosomal compartment in transfected or injected cells, both in vitro and in vivo (Godbey, W, et al 1999, Proc Natl Acad Sci USA 96: 5177-5181; and Lechardeur, D, et al 1999, Gene Ther 6: 482-497; and references cited therein). Accordingly, in certain embodiments, DNA uptake is enhanced by the receptor-mediated endocytosis pathway using transferrin-polylysine conjugates or adenoviral-mediated vesicle disruption to effect the release of DNA from endosomes. However, the invention is not limited to this or any other theory or mechanism of operation referred to herein.

Buffering the endosomal pH using endosomal-scaping elements also protects DNA from degradation (Kircheis, R, et al 2001, Adv Drug Deliv Rev 53: 341-358; Boussif, 0, et al 1995, Proc Natl Acad Sci USA 92: 7297-7301; and Pollard, H, et al 1998, J Biol Chem 273: 7507-7511; and references cited therein). Thus, in certain embodiments, DNA complexes are delivered with polycations or cationic polymers that possess substantial buffering capacity below physiological pH, such as polyethylenimine, lipopolyamines and polyamidoamine polymers. In certain embodiments, DNA condensing compounds, such as the ones described above, are combined with viruses (Curiel, D, et al Proc Natl Acad Sci USA 88: 8850-8854, 1991; Wagner, E, et al Proc Natl Acad Sci USA 89: 6099-6103, 1992 and Cotten, M, et al, 1992, Proc Natl Acad Sci USA 89: 6094-6098), viral peptides (Wagner, E, et al 1992, Proc Natl Acad Sci USA 89: 7934-7938; Plank, C, et al 1994, J Biol Chem 269: 12918-12924) and subunits of toxins (Uherek, C, et al, 1998, J Biol Chem 273: 8835-48). These materials significantly enhance the release of DNA from endosomes. In certain embodiments, viruses, viral peptides, toxins or subunits of toxins may be coupled to DNA/polylysine complexes via biochemical means or specifically by a streptavidin-biotin bridge (Wagner et al, 1992, Proc. Natl. Acad. Sci. USA 89:6099-6103; Plank et al, 1994, J. Biol. Chem. 269(17):12918-12924). In other certain embodiments, the virus that is complexed with the DNA may be adenovirus, retrovirus, vaccinia virus, or parvovirus. The viruses may be linked to PEI or another cationic polymer associated with the nucleic acid. In certain embodiments, the virus may be alphavirus, orthomyxovirus, or picornavirus. In certain embodiments, the virus is defective or chemically inactivated. The virus may be inactivated by short-wave UV radiation or the DNA intercalator psoralen plus long-wave UV. The adenovirus may be coupled to polylysine, either enzymatically through the action of transglutaminase or biochemically by biotinylating adenovirus and streptavidinylating the polylysine moiety. Transferrin may also be useful in combination with cationic polymers, adenoviruses and/or other materials disclosed herein to produce transgenic avians. For example, DNA complexes containing PEI, PEI-modified transferrin, and PEI-bound influenza peptides may be used to enhance transgenic avian production.

In other certain embodiments, complexes containing plasmid DNA, transferrin-PEI conjugates, and PEI-conjugated peptides derived from the N-terminal sequence of the influenza virus hemagglutinin subunit HA-2 may be used to produce transgenic chickens. In certain embodiments, the PEI-conjugated peptide may be an amino-terminal amino acid sequence of influenza virus hemagglutinin which may be elongated by an amphipathic helix or by carboxyl-terminal dimerization.

The present invention provides for methods of dispersing or distributing nucleic acid in a cell, for example, in an avian cell. The avian cell may be, for example, and without limitation, a cell of a stage I avian embryo, a cell of a stage II avian embryo, a cell of a stage III avian embryo, a cell of a stage IV avian embryo, a cell of a stage V avian embryo, a cell of a stage VI avian embryo, a cell of a stage VII avian embryo, a cell of a stage VIII avian embryo, a cell of a stage IX avian embryo, a cell of a stage X avian embryo, a cell of a stage XI avian embryo or a cell of a stage XII avian embryo. In one particularly useful embodiment, the avian cell is a cell of a stage X avian embryo.

In one aspect of the present invention, cationic polymers are useful to distribute, for example, homogeneously distribute, nucleic acid introduced into a cell, for example, an embryonic avian cell. The present invention contemplates the use of cationic polymers including, but not limited to, those disclosed herein.

However, substances other than cationic polymers also capable of distributing or dispersing nucleic acids in a cell are included within the scope of the present invention.

The concentration of cationic polymer used is not critical though, in one useful embodiment, enough cationic polymer is present to coat the nucleic acid to be introduced into the avian cell. The cationic polymer may be present in an aqueous mixture with the nucleic acid to be introduced into the cell at a concentration in a range of an amount equal to about the weight of the nucleic acid to a concentration wherein the solution is saturated with cationic polymer. In one useful embodiment, the cationic polymer is present in an amount in a range of about 0.01% to about 50%, for example, about 0.1% to about 20% (e.g., about 5%). The molecular weights of the cationic polymers can range from a molecular weight of about 1,000 to a molecular weight of about 1,000,000. In one embodiment, the molecular weight of the cationic polymers range from about 5,000 to about 100,000 for example, about 20,000 to about 30,000.

In one particularly useful aspect of the invention, procedures that are effective to facilitate the production of a transgenic avian may be combined to provide for an enhanced production of a transgenic avian wherein the enhanced production is an improved production of a transgenic avian relative to the production of a transgenic avian by only one of the procedures employed in the combination. For example, one or more of integrase activity, NLS, cationic polymer or other technique useful to enhance transgenic avian production disclosed herein can be used in the same procedure to provide for an enhanced production of transgenic avians relative to an identical procedure which does not employ all of the same techniques useful to enhance transgenic avian production.

Another aspect of the present invention is a vertebrate animal cell which has been genetically modified with a transgene vector according to the present invention and as described herein. For example, in one embodiment, the transformed cell can be a chicken early stage blastodermal cell or a genetically transformed cell line, including a sustainable cell line. The transfected cell according to the present invention may comprise a transgene stably integrated into the nuclear genome of the recipient cell, thereby replicating with the cell so that each progeny cell receives a copy of the transfected nucleic acid. A particularly useful cell line for the delivery and integration of a transgene comprises a heterologous attP site that can increase the efficiency of integration of a polynucleotide by phiC31 integrase and, optionally, a region for expressing the integrase.

A retroviral vector can be used to deliver a recombination site such as an att site into the cellular genomes, such as avian genomes, since an attP or attB site is less than 300 bp. For example, the attP site can be inserted into the NLB retroviral vector, which is based on the avian leukosis virus genome. A lentiviral vector is a particularly suitable vector because lentiviral vectors can transduce non-dividing cells, so that a higher percentage of cells will have an integrated attP site.

The lacZ region of NLB is replaced by the attP sequence. A producer cell line would be created by transformation of, for example, the Isolde cell line capable of producing a packaged recombinant NLB-attP virus pseudo-typed with the envA envelope protein. Supernatant from the Isolde NLB-attP line is concentrated by centrifugation to produce high titer preparations of the retroviral vector that can then be used to deliver the attP site to the genome of a cell, for example, as described in Example 9 below.

In one embodiment, an attP-containing line of transgenic birds are a source of attP transgenic embryos and embryonic cells. Fertile zygotes and oocytes bearing a heterologous attP site in either the maternal, paternal, or both, genomes can be used for transgenic insertion of a desired heterologous polynucleotide. A transgene vector bearing an attB site, for example, would be injected into the cytoplasm along with either an integrase expression plasmid, mRNA encoding the integrase or the purified integrase protein. The oocyte or zygote is then cultured to hatch by ex ovo methods or reintroduced into a recipient hen such that the hen lays a hard shell egg the next day containing the injected egg.

In another example, fertile stage I to XII embryos, for example, stage VII to XII embryos, hemizygous or homozygous for the heterologous integration site, for example, the attP sequence, may be used as a source of blastodermal cells. The cells are harvested and then transfected with a transgene vector bearing a second recombination site, such as an attB site, plus a nucleotide sequence of interest along with a source of integrase. The transfected cells are then injected into the subgerminal cavity of windowed fertile eggs. The chicks that hatch will bear the nucleotide sequence of interest and the second integration site integrated into the attP site in a percentage of their somatic and germ cells. To obtain fully transgenic birds, chicks are raised to sexual maturity and those that are positive for the transgene in their semen are bred to non-transgenic mates. As disclosed herein, in certain embodiments, the cells of the invention, e.g., embryos, may include an integrase which specifically recognizes recombination sites and which is introduced into cells containing a nucleic acid construct of the invention under conditions such that the nucleic acid sequence(s) of interest will be inserted into the nuclear genome. Methods for introducing such an integrase into a cell are described herein. In some embodiments, the site-specific integrase is introduced into the cell as a polypeptide. In alternative embodiments, the site-specific integrase is introduced into the transgenic cell as a polynucleotide encoding the integrase, such as an expression cassette optionally carried on a transient expression vector, and comprising a polynucleotide encoding the recombinase.

In one embodiment, the invention is directed to methods of using a vector for site-specific integration of a heterologous nucleotide sequence into the genome of a cell, the vector comprising a circular backbone vector, a polynucleotide of interest operably linked to a promoter, and a first recombination site, wherein the genome of the cell comprises a second recombination site and recombination between the first and second recombination sites is facilitated by an integrase. In certain embodiments, the integrase facilitates recombination between a bacterial genomic recombination site (attB) and a phage genomic recombination site (attP).

In another embodiment, the invention is directed to a cell having a transformed genome comprising an integrated heterologous polynucleotide of interest whose integration, mediated by an integrase, was into a recombination site native to the cell genome and the integration created a recombination-product site comprising the polynucleotide sequence. In yet another embodiment, integration of the polynucleotide was into a recombination site not native to the cell genome, but instead into a heterologous recombination site engineered into the cell genome.

In further embodiments, the invention is directed to transgenic vertebrate animals, such as transgenic birds, comprising a modified cell and progeny thereof as described above, as well as methods of producing the same.

For example, cells genetically modified to carry a heterologous attB or attP site by the methods of the present invention can be maintained under conditions that, for example, keep them alive but do not promote growth and/or cause the cells to differentiate or dedifferentiate. Cell culture conditions may be permissive for the action of the integrase in the cells, although regulation of the activity of the integrase may also be modulated by culture conditions (e.g., raising or lowering the temperature at which the cells are cultured).

The present invention also provides for methods of purifying artificial chromosomes. In one significant embodiment of the invention, the purified artificial chromosomes are used to produce transchromosomic animals including, but not limited to, transchromosomic avians (e.g., transchromosomic chickens). Any useful type of artificial chromosome is contemplated for use in the present invention.

In one aspect, the present invention is directed to purifying artificial chromosomes useful in producing transgenic avians (e.g., chickens) by tagging the chromosomes with a marker dye, for example, and without limitation, a fluorescent marker dye. In one particularly useful aspect of the invention, sequence specific polyamide probes are used in the tagging process. The tagged chromosomes may be purified by methods which provide for the discrimination of the tagged chromosomes over untagged chromosomes, such as flow cytometry.

For example, the method of Gygi et al (2002) Nucleic Acids Res. 30: 2790-2799, the disclosure of which is incorporated by reference herein in its entirety, is contemplated for use in the present invention. Briefly, the protocol provides for the use of synthetic polyamide probes to fluorescently label regions on the artificial chromosomes (e.g., heterochromatin in the case of SATACs) which are then isolated by flow cytometry. The polyamides may bind to the minor groove of DNA of the chromosomes in a sequence specific manner without the need to disrupt the chromosome (e.g., denature the DNA).

Any useful region (e.g., nucleotide sequence or sequences) of the artificial chromosome to be purified can be tagged using probes as disclosed herein. For example, any sequence present in the artificial chromosome to be purified and not present, or present to a lesser degree, in one or more chromosomes naturally occurring in the host cell may be tagged using a probe. For example, telomeric regions, centromeric regions, non-coding regions and/or coding regions of the artificial chromosome may be targeted by the probes thereby tagging the artificial chromosome. For example, the heterochromatic region of SATACs can be targeted for tagging since SATACs (or megachromosomes) (see, for example, U.S. Pat. No. 6,077,697, issued Jun. 20, 2000) are comprised primarily of heterochromatic DNA (e.g., repeat sequences of the mouse major satellite DNA sequences). For example, fluorescent in situ hybridization utilizing probes designed to recognize mouse major satellite sequences produce an intense fluorescent signal throughout the length of the heterochromatic region of SATACs. The signal has been shown not to be present in background chromosomes of non-mouse cell lines such as ChY1 cells showing that cell lines such as ChY1 are useful to host the artificial chromosome when targeting the artificial chromosome for tagging (e.g., tagging with labeled probes).

The invention contemplates the purification of any artificial chromosome useful for the production of transchromosomic animals (e.g., transchromosomic avians) as disclosed herein. For example, artificial chromosomes and methods related thereto such as those disclosed in U.S. Pat. No. 6,025,155 issued Feb. 15, 2000; U.S. Pat. No. 6,743,967 issued Jun. 1, 2004; U.S. Pat. No. 6,077,697 issued Jun. 20, 2000; U.S. Pat. No. 5,288,625 issued Feb. 22, 1994; U.S. Pat. No. 5,721,118 issued Feb. 24, 1998; U.S. Pat. No. 6,133,503 issued Oct. 17, 2000; US patent publication 2003/0113917 published Jun. 19, 2003; US patent publication 2003/0003435 published Jan. 2, 2003; WO 95/32297, International Publication Date Nov. 30, 1995 are contemplated for use in accordance with the present invention. The disclosures of each of these six US patents, two published US patent applications and one published WO patent application are incorporated in their entirety herein by reference. In addition, the following publications disclose artificial chromosomes or methods related thereto which are contemplated for use in the present invention. Each of these publications is incorporated herein in its entirety by reference: Bower, “Constructing a fully defined human minichromosome: Cloning a centromere” (1987) Proc. 4th Eur. Congress Biotechnol. 3:571; Carine, et al., “Chinese hamster cells with a minichromosome containing centromere region of human chromosome 1” (1986) Somatic Cell Molec. Genet. 12:479-491; Carine, et al., “Molecular characterization of human minichromosomes with centromere from chromosome 1 in hamster-human hybrids” (1989) Somatic Cell Molec. Genet. 15(15):445-160; Farr, et al. “Generation of a human X-derived minichromosome using telomere-associated chromosome fragmentation” (1995) EMBO J. 14:5444-5454; Hadlaczky, et al, Centromere formation in mouse cells cotransformed with human DNA and a dominant marker gene (1991) Proc. Natl. Acad. Sci. USA, 88:8106-8110; Hadlaczky, et al, Satellite DNA-based artificial chromosomes for use in gene therapy (2001) Curr. Opin. Mol. Ther., Apr. 3(2):125-32; Hadlaczky and Szalay, “Mammalian artificial chromosomes: Potential vectors for gene therapy” (1996) Abstract from International Symposium on Gene Therapy of Cancer, AIDS and Genetic Disorders, Trieste (Italy) (April, 10-13); Hadlaczky and Szalay, “Mammalian artificial chromosomes: Introduction of novel genes into mammalian artificial chromosomes” (1996) Abstract from International Symposium on Gene Therapy of Cancers AIDS and Genetic Disorders, Trieste (Italy) (Apr. 10-13,); Harrington et al., Formation of de novo centromeres and construction of first-generation human artificial microchromosomes (1997) Nature Genetics, 15: (4) 345-355; Heller et al. Mini-chromosomes derived from the human Y chromosome by telomere directed chromosome breakage (1996) Proc. Natl. Acad. Sci. USA, 93:7125-7130; Huxley, “Mammalian artificial chromosomes: a new tool for gene therapy” (1994) Gene Therapy, 1:7-12; Huxley, C., Mammalian artificial chromosomes and chromosome transgenics (1997) Trends Genet., September; 13(9):345-7; Kereso, et al, De novo chromosome formations by large-scale amplification of the centromeric region of mouse chromosomes, Chromosome Res. (1996) April; 4(3):226-239; Katoh, et al, Construction of a novel human artificial chromosome vector for gene delivery (2004) Biochem. Biophys. Res. Commun. Aug. 20; 321(2):280-290; Larin, et al., Advances in human artificial chromosome technology (2002) Trends Genet., June; 18 (6):313-9; Lipps, et al, Chromosome-based vectors for gene therapy (2003) Gene, 304:23-33; Mills, et al, Generation of an 2.4 Mb human X centromere-based minichromosome by targeted telomere-associated chromosome fragmentation in DT40 (1999) Human Molecular Genetics, 8(5) 751-761; Murray, et al., “Construction of artificial chromosomes in yeast” (1983) Nature 305:189-193; Masumoto, Structural and functional analyses of the centromere of human chromosome 21: construction of human artificial chromosomes (2001) Tanpakushitsu Kakusan Koso., December; 46(16 Suppl):2375-8; Praznovszky, et al, De novo chromosome formation in rodent cells (1991) Proc. Natl. Acad. Sci. USA, 88:11042-11046; Raimondi, et al., “X-ray mediated size reduction, molecular characterization and transfer in model systems of a human artificial minichromosome” (1996) Abstract from International Symposium on Gene Therapy of Cancer, AIDS and Genetic Disorders, Trieste (Italy) Apr. 10-13; Robi, et al., Artificial chromosome vectors and expression of complex proteins in transgenic animals (2003) Theriogenology, January 1; 59(1):107-13; Telenius, et al, Stability of a functional murine satellite DNA-based artificial chromosome across mammalian species (1999) Chromosome Research, 7:3-7; and Wang, et al, Expression of a Reporter Gene After Microinjection of Mammalian Artificial Chromosomes into Pronuclei of Bovine Zygotes (2001) Mol. Reprod. and Dev. 60:433-438.

In one useful embodiment of the invention, labeled (e.g., fluorescently labeled) polyamide probes are employed to tag the artificial chromosomes to facilitate purification (e.g., flow cytometry based purification) of the artificial chromosome. Polyamide probes may be prepared by any useful method known in the art such as those methods disclosed in: PCT/US97/12733; PCT/US97/03332; PCT/US97/12722; PCT/US98/06997; PCT/US98/02444; PCT/US98/02684; PCT/US98/01006; PCT/US98/03829; PCT/US98/0714 and U.S. Pat. No. 6,673,940, issued Jan. 6, 2004. The disclosures of each of these nine PCT applications and one issued patent are incorporated in their entirety herein by reference. Other useful methods included in the following references are contemplated for use in accordance with the present invention: U.S. Pat. No. 6,673,940, issued Jan. 6, 2004; U.S. Pat. No. 6,555,692, issued Apr. 29, 2003; U.S. Pat. No. 6,506,906, issued Jan. 14, 2003; U.S. Pat. No. 6,472,537, issued Oct. 29, 2002; U.S. Pat. No. 6,432,638, issued Aug. 13, 2002; U.S. Pat. No. 6,303,312, issued Oct. 16, 2001; and U.S. Pat. No. 6,143,901, issued Nov. 7, 2000. The disclosures of each of these seven issued patents are incorporated in their entirety herein by reference. In addition, the following five publications, the disclosures of which are incorporated in their entirety herein by reference, disclose compositions and methods which are contemplated for use in the present invention: Dervan, Molecular Recognition of DNA by Small Molecules (2001) Bioorganic & Medicinal Chem. 9; 2215-2235; Gygi, et al., Use of Fluorescent Sequence-Specific Polyamides to Discriminate Human Chromosomes by Microscopy and Flow Cytometry (2002) Nucleic Acids Research; 30: (13) 2790-2799; Rucker, et al., Sequence Specific Fluorescence Detection of Double Strand DNA (2003); J. Am. Chem. Soc.; 125: 1195-1202; Recognition of the DNA minor groove by pyrrole-imidazole polyamides (2003) Curr Opin Struct Biol 13: 284-99; and J Am Chem Soc (2003) 125: 1195-202.

Wade, et al. (J. Am. Chem. Soc., vol. 114:8783-8794 (1992)) reported the design of polyamides that bind in the minor groove of dsDNA at 5′-(A,T)G(A,T)C(A,T)-3′ sequences by a dimeric, side-by-side motif. Mrksich, et al. (Proc. Natl. Acad. Sci. USA, vol. 89:7586-7590 (1992)), reported an antiparallel, side-by-side polyamide motif for sequence-specific recognition in the minor groove of dsDNA by the designed peptide 1-methylimidazole-2-carboxamide netropsin. Trauger, et al. (Nature, vol. 382:559-561 (1996)) reports the recognition of a targeted dsDNA by a polyamide at subnanomolar concentrations. The disclosure of each of these three publications is incorporated by reference herein in its entirety.

In one embodiment, the particular order of amino acids in polyamides useful for making labeled (e.g., fluorescently labeled) polyamide probes, and their pairing in dimeric, antiparallel complexes formed by association of two polyamide polymers can be used to determine the sequence of nucleotides in dsDNA with which the polymers preferably associate.

The development of pairing rules for minor groove binding polyamides derived from N-methylpyrrole (Py) and N-methylimidazole (Im) amino acids can provide a useful code to control target nucleotide base pair sequence specificity. Specifically, in one embodiment an Im/Py pair in adjacent polymers can distinguish G-C from C-G and both of these from A-T or T-A base pairs. A Py/Py pair can specify A-T from G-C but cannot distinguish A-T from T-A. White, et al. (Biochemistry, vol. 35:12532-12537 (1996), the disclosure of which is incorporated in its entirety herein by reference) reported the effects of the A-T/T-A degeneracy of Py/Im polyamide recognition in the minor groove of dsDNA. White, et al. (Chem. & Biol. vol. 4:569-578 (1997), the disclosure of which is incorporated in its entirety herein by reference) reported the pairing rules for recognition in the minor groove of dsDNA by Py/Im polyamides and the 5′ to 3′, N to C orientation preference for polyamide binding in the minor groove of dsDNA.

The inclusion of an aromatic amino acid, such as 3-hydroxy-N-methylpyrrole (Hp)(made by replacing a single hydrogen atom in Py with a hydroxy group), in a polyamide and paired opposite Py enables A-T to be discriminated from T-A by an order of magnitude. Utilizing Hp together with Py and Im in polyamides provides a code to distinguish all four Watson-Crick base pairs (i.e., A-T, T-A, G-C, and C-G) in the minor groove of dsDNA, as follows:

Pairing Code
for Minor Groove Recognition

Pair

| | G-C | C-G | T-A | A-T |
|---|---|---|---|---|
| Im/Py | + | − | − | − |
| Py/Im | − | + | − | − |
| Hp/Py | − | − | + | − |
| Py/Hp | − | − | − | + |

Favored (+)
Disfavored (−)

It is understood that the method of designing and making probes use as disclosed herein is unimportant and present invention is not limited to any particular probe or to any particular polyamide probe or method of making such probe which is used to purify artificial chromosomes for the production of transchromosomic animals, such as transchromosomic avians as disclosed herein.

One aspect of the invention are methods for generating a genetically modified cell for example, an avian cell, and progeny thereof, using a tagged chromosome. The methods may include providing an isolated modified chromosome comprising a lac operator region and a first recombination site, delivering the modified chromosome to an avian cell, thereby generating a trisomic or transchromosomic avian cell, delivering to the avian cell a source of a tagged polypeptide comprising a fluorescent domain and a lac repressor domain, delivering a source of integrase activity to the avian cell, delivering a polynucleotide comprising a second recombination site and a region encoding a polypeptide to the avian cell, maintaining the avian cell under conditions suitable for the integrase to mediate recombination between the first and second recombination sites, thereby integrating the polynucleotide into the modified chromosome and generating a genetically modified avian cell, expressing the tag polypeptide by the avian cell, allowing the tag polypeptide to bind to the modified chromosome so as to label the modified chromosome, and isolating the modified chromosome by selecting modified chromosomes having a tag polypeptide bound thereto.

In one embodiment of the invention, the second avian cell is selected from the group consisting of a stage VII-XII blastodermal cell, a stage I embryo, a stage X embryo; an isolated primordial germ cell, an isolated non-embryonic cell, and an oviduct cell.

In various embodiments, the isolated modified chromosome is an avian chromosome or an artificial chromosome.

In other embodiments of the invention, the step of providing an isolated modified chromosome comprising a lac operator region and a first recombination site comprises the steps of generating a trisomic or transchromosomic avian cell by delivering to an isolated avian cell an isolated chromosome and a polynucleotide comprising a lac operator and a second recombination site, maintaining the trisomic or transchromosomic cell under conditions whereby the heterologous polynucleotide is integrated into the chromosome by homologous recombination, delivering to the avian cell a source of a tag polypeptide to label the chromosome, and isolating the labeled chromosome.

In one embodiment of the invention, the lac operator region is a concatamer of lac operators. In other embodiments of the invention, the tag polypeptide is expressed from an expression vector.

In one embodiment of the invention, the tag polypeptide is microinjected into the cell. In various embodiments of the invention, the method of delivery of a chromosome to an avian cell is selected from the group consisting of liposome delivery, microinjection, microcell, electroporation and gene gun delivery, or a combination thereof.

In embodiments of the invention, the fluorescent domain of the tag polypeptide is GFP.

In one embodiment of the invention, the method further comprises the step of delivering the second avian cell to an avian embryo. The embryo may be maintained under conditions suitable for hatching as a chick.

In one embodiment of the invention, the second avian cell is maintained under conditions suitable for the proliferation of the cell, and progeny thereof.

In various embodiments of the invention, the source of integrase activity is delivered to a first avian cell as a polypeptide or expressed from a polynucleotide, said polynucleotide being selected from an mRNA and an expression vector.

In one embodiment of the invention, the tag polypeptide activity is delivered to the avian cell as a polypeptide or expressed from a polynucleotide operably linked to a promoter. In another embodiment of the invention, the promoter is an inducible promoter. In yet another embodiment of the invention, the integrase is phiC31 integrase and in various embodiments of the invention, the first and second recombination sites are selected from an attB and an attP site, but wherein the first and second sites are not identical.

Other aspects of the present invention include methods of expressing a heterologous polypeptide in vertebrate cells by stably transfecting cells using site-specific integrase-mediation and a recombinant nucleic acid molecule, as described herein, and culturing the transfected cells under conditions suitable for expression of the heterologous polypeptide. In addition, the present invention includes methods of expressing a heterologous polypeptide in a transgenic vertebrate animal by producing a transgenic vertebrate animal using methods known in the field or described herein in combination with using site-specific integration of nucleic acid molecules as described herein, and exposing the animal to conditions suitable for expression of the heterologous polypeptide.

The protein of the present invention may be produced in purified form by any known conventional techniques. For example, in the case of heterologous protein production in eggs, the egg white may be homogenized and centrifuged. The supernatant may then be subjected to sequential ammonium sulfate precipitation and heat treatment. The fraction containing the protein of the present invention is subjected to gel filtration in an appropriately sized dextran or polyacrylamide column to separate the proteins. If necessary, the protein fraction may be further purified by HPLC or other methods well known in the art of protein purification.

The methods of the invention are useful for expressing nucleic acid sequences that are optimized for expression in the host cells and which encode desired polypeptides or derivatives and fragments thereof. Derivatives include, for instance, polypeptides with conservative amino acid replacements, that is, those within a family of amino acids that are related in their side chains (commonly known as acidic, basic, nonpolar, and uncharged polar amino acids). Phenylalanine, tryptophan, and tyrosine are sometimes classified jointly as aromatic amino acids and other groupings are known in the art (see, for example, “Biochemistry”, 2nd ed, L. Stryer, ed., W.H. Freeman & Co., 1981). Peptides in which more than one replacement has taken place can readily be tested for activity in the same manner as derivatives with a single replacement, using conventional polypeptide activity assays (e.g. for enzymatic or ligand binding activities).

Regarding codon optimization, if the recombinant nucleic acid molecules are transfected into a recipient chicken cell, the sequence of the nucleic acid insert to be expressed can be optimized for chicken codon usage. This may be determined from the codon usage of at least one, or more than one, protein expressed in a chicken cell according to well known principles. For example, in the chicken the codon usage could be determined from the nucleic acid sequences encoding the proteins such as lysozyme, ovalbumin, ovomucin and ovotransferrin of chicken. Optimization of the sequence for codon usage can elevate the level of translation in avian eggs.

The present invention provides methods for the production of a protein by cells comprising the steps of maintaining a cell, transfecting with a first expression vector and, optionally, a second expression vector, under conditions suitable for proliferation and/or gene expression and such that an integrase will mediate site specific recombination at att sites. The expression vectors may each have a transcription unit comprising a nucleotide sequence encoding a heterologous polypeptide, wherein one polypeptide is an integrase, a transcription promoter, and a transcriptional terminator. The cells may then be maintained under conditions for the expression and production of the desired heterologous polypeptide(s).

The present invention further relates to methods for gene expression by cells, such as avian cells, from nucleic acid vectors, and transgenes derived therefrom, that include more than one polypeptide-encoding region wherein, for example, a first polypeptide-encoding region can be operatively linked to an avian promoter and a second polypeptide-encoding region is operatively linked to an Internal Ribosome Entry Sequence (IRES). It is contemplated that the first polypeptide-encoding region, the IRES and the second polypeptide-encoding region of a recombinant DNA of the present invention may be arranged linearly, with the IRES operably positioned immediately 5′ of the second polypeptide-encoding region. This nucleic acid construct can be used for the production of certain proteins in vertebrate animals or in their cells. For example, when inserted into the genome of an avian cell or a bird and expressed therein, will generate individual polypeptides that may be post-translationally modified and combined in the white of a hard shell bird egg. Alternatively, the expressed polypeptides may be isolated from an avian egg and combined in vitro.

The invention, therefore, includes methods for producing multimeric proteins including immunoglobulins, such as antibodies, and antigen binding fragments thereof. Thus, in one embodiment of the present invention, the multimeric protein is an immunoglobulin, wherein the first and second heterologous polypeptides are immunoglobulin heavy and light chains respectively. Illustrative examples of this and other aspects of the present invention for the production of heterologous multimeric polypeptides in avian cells are fully disclosed in U.S. patent application Ser. No. 09/877,374, filed Jun. 8, 2001, and U.S. patent application Ser. No. 10/251,364, filed Sep. 18, 2002, now issued U.S. Pat. No. 7,312,374 both of which are incorporated herein by reference in their entirety.

Accordingly, the invention further provides immunoglobulin and other multimeric proteins that have been produced by transgenic vertebrates including avians of the invention.

In various embodiments, an immunoglobulin polypeptide encoded by the transcriptional unit of at least one expression vector may be an immunoglobulin heavy chain polypeptide comprising a variable region or a variant thereof, and may further comprise a D region, a J region, a C region, or a combination thereof. An immunoglobulin polypeptide encoded by an expression vector may also be an immunoglobulin light chain polypeptide comprising a variable region or a variant thereof, and may further comprise a J region and a C region. The present invention also contemplates multiple immunoglobulin regions that are derived from the same animal species, or a mixture of species including, but not only, human, mouse, rat, rabbit and chicken. In certain embodiments, the antibodies are human or humanized.

In other embodiments, the immunoglobulin polypeptide encoded by at least one expression vector comprises an immunoglobulin heavy chain variable region, an immunoglobulin light chain variable region, and a linker peptide thereby forming a single-chain antibody capable of selectively binding an antigen.

Examples of therapeutic antibodies that may be produced in methods of the invention include but are not limited to HERCEPTIN™ (Trastuzumab) (Genentech, CA) which is a humanized anti-HER2 monoclonal antibody for the treatment of patients with metastatic breast cancer; REOPRO™ (abciximab) (Centocor) which is an anti-glycoprotein IIb/IIIa receptor on the platelets for the prevention of clot formation; ZENAPAX™ (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primate anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (CS) antibody (Alexion Pharm); D2E7 is a humanized anti-TNF-α antibody (CATIBASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

The invention can be used to express, in large yields and at low cost, a wide range of desired proteins including those used as human and animal pharmaceuticals, diagnostics, and livestock feed additives. Proteins such as fusion proteins, growth hormones, cytokines, structural proteins and enzymes including human growth hormone, interferon, lysozyme, and β-casein are examples of proteins which are desirably expressed in the oviduct and deposited in eggs according to the invention. Other possible proteins to be produced include, but are not limited to, albumin, α-1 antitrypsin, antithrombin III, collagen, factors VIII, IX, X (and the like), fibrinogen, hyaluronic acid, insulin, lactoferrin, protein C, erythropoietin (EPO), granulocyte colony-stimulating factor (G-CSF), granulocyte macrophage colony-stimulating factor (GM-CSF), tissue-type plasminogen activator (tPA), feed additive enzymes, somatotropin, and chymotrypsin. Immunoglobulins (shown, for example in Example 10 below) and genetically engineered antibodies, including immunotoxins which bind to surface antigens on human tumor cells and destroy them, can also be expressed for use as pharmaceuticals or diagnostics.

Other specific examples of therapeutic proteins which are contemplated for production as disclosed herein include, with out limitation, factor VIII, b-domain deleted factor VIII, factor VIIa, factor IX, anticoagulants; hirudin, alteplase, tpa, reteplase, tpa, tpa-3 of 5 domains deleted, insulin, insulin lispro, insulin aspart, insulin glargine, long-acting insulin analogs, hgh, glucagons, tsh, follitropin-beta, fsh, gm-csf, pdgh, ifn alpa2a, inf-apha, inf-beta 1b, differs from h protein by c17 to s, ifn-beta 1a, ifn-gamma1b, il-2, il-11, hbsag, ospa, murine mab directed against t-lymphocyte antigen, murine mab directed against tag-72, tumor-associated glycoprotein, fab fragments derived from chimeric mab, directed against platelet surface receptor gpII(b)/III(a), murine mab fragment directed against tumor-associated antigen ca125, murine mab fragment directed against human carcinoembryonic antigen, cea, murine mab fragment directed against human cardiac myosin, murine mab fragment directed against tumor surface antigen psma, murine mab fragments (fab/fab2 mix) directed against hmw-maa, murine mab fragment (fab) directed against carcinoma-associated antigen, mab fragments (fab) directed against nca 90, a surface granulocyte nonspecific cross reacting antigen, chimeric mab directed against cd20 antigen found on surface of b lymphocytes, humanized mab directed against the alpha chain of the il2 receptor, chimeric mab directed against the alpha chain of the il2 receptor, chimeric mab directed against tnf-alpha, humanized mab directed against an epitope on the surface of respiratory synctial virus, humanized mab directed against her 2, i.e., human epidermal growth factor receptor 2, human mab directed against cytokeratin tumor-associated antigen anti-ctla4, chimeric mab directed against cd 20 surface antigen of b lymphocytes dornase-alpha dnase, beta glucocerebrosidase, tnf-alpha, il-2-diptheria toxin fusion protein, tnfr-lgg fragment fusion protein laronidase, dnaases, alefacept, darbepoetin alfa (colony stimulating factor), tositumomab, murine mab, alemtuzumab, rasburicase, agalsidase beta, teriparatide, parathyroid hormone derivatives, adalimumab (lggl), anakinra, biological modifier, nesiritide, human b-type natriuretic peptide (hbnp), colony stimulating factors, pegvisomant, human growth hormone receptor antagonist, recombinant activated protein c, omalizumab, immunoglobulin e (lge) blocker and lbritumomab tiuxetan.

In various embodiments of the transgenic vertebrate animal of the present invention, the expression of the transgene may be restricted to specific subsets of cells, tissues or developmental stages utilizing, for example, trans-acting factors acting on the transcriptional regulatory region operably linked to the polypeptide-encoding region of interest of the present invention and which control gene expression in the desired pattern. Tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Another aspect of the present invention provides a method for the production of a heterologous protein capable of forming an antibody suitable for selectively binding an antigen. This method comprises a step of producing a transgenic vertebrate animal incorporating at least one transgene, the transgene encoding at least one heterologous polypeptide selected from an immunoglobulin heavy chain variable region, an immunoglobulin heavy chain comprising a variable region and a constant region, an immunoglobulin light chain variable region, an immunoglobulin light chain comprising a variable region and a constant region, and a single-chain antibody comprising two peptide-linked immunoglobulin variable regions.

In one embodiment of this method, the isolated heterologous protein is an antibody capable of selectively binding to an antigen and which may be generated by combining at least one immunoglobulin heavy chain variable region and at least one immunoglobulin light chain variable region, for example, cross-linked by at least one disulfide bridge. The combination of the two variable regions generates a binding site that binds an antigen using methods for antibody reconstitution that are well known in the art.

The present invention also encompasses immunoglobulin heavy and light chains, or variants or derivatives thereof, to be expressed in separate transgenic avians, and thereafter isolated from separate media including serum or eggs, each isolate comprising one or more distinct species of immunoglobulin polypeptide. The method may further comprise the step of combining a plurality of isolated heterologous immunoglobulin polypeptides, thereby producing an antibody capable of selectively binding to an antigen. In this embodiment, for instance, two or more individual transgenic avians may be generated wherein one transgenic produces serum or eggs having an immunoglobulin heavy chain variable region, or a polypeptide comprising such, expressed therein. A second transgenic animal, having a second transgene, produces serum or eggs having an immunoglobulin light chain variable region, or a polypeptide comprising such, expressed therein. The polypeptides from two or more transgenic animals may be isolated from their respective sera and eggs and combined in vitro to generate a binding site capable of binding an antigen.

One aspect of the present invention, therefore, concerns transgenic vertebrate animals such as transgenic birds, for example, transgenic chickens, comprising a recombinant nucleic acid molecule and which may (though optionally) expresses a heterologous gene in one or more cells in the animal. Suitable methods for the generation of transgenic animals are known in the art and are described in, for example, WO 99/19472, published Apr. 22, 1999; WO 00/11151, published Mar. 2, 2000; and WO 00/56932, published Sep. 28, 2000, the disclosures of which are incorporated herein by reference in their entirety.

Embodiments of the methods for the production of a heterologous polypeptide by avian tissue such as oviduct tissue and the production of eggs which contain heterologous protein involve providing a suitable vector and introducing the vector into embryonic blastodermal cells together with an integrase, for example, a serine recombinase such as phiC31 integrase, so that the vector can integrate into the avian genome. A subsequent step involves deriving a mature transgenic avian from the transgenic blastodermal cells produced in the previous steps. Deriving a mature transgenic avian from the blastodermal cells optionally involves transferring the transgenic blastodermal cells to an embryo and allowing that embryo to develop fully, so that the cells become incorporated into the bird as the embryo is allowed to develop.

Another alternative may be to transfer a transfected nucleus to an enucleated recipient cell which may then develop into a zygote and ultimately an adult bird. The resulting chick is then grown to maturity.

In another embodiment, the cells of a blastodermal embryo are transfected or transduced with the vector and integrase directly within the embryo. It is contemplated, for example, that the recombinant nucleic acid molecules of the present invention may be introduced into a blastodermal embryo by direct microinjection of the DNA into a stage X or earlier embryo that has been removed from the oviduct. The egg is then returned to the bird for egg white deposition, shell development and laying. The resulting embryo is allowed to develop and hatch, and the chick allowed to mature.

In one embodiment, a transgenic bird of the present invention is produced by introducing into embryonic cells such as, for instance, isolated avian blastodermal cells, a nucleic acid construct comprising an attB recombination site capable of recombining with a pseudo-attP recombination site found within the nuclear genome of the organism from which the cell was derived, and a nucleic acid fragment of interest, in a manner such that the nucleic acid fragment of interest is stably integrated into the nuclear genome of germline cells of a mature bird and is inherited in normal Mendelian fashion. It is also within the scope of the invention that the targeted cells for receiving the transgene have been engineered to have a heterologous attP recombination site, or other recombination site, integrated into the nuclear genome of the cells, thereby increasing the efficiency of recognition and recombination with a heterologous attB site.

In either case, the transgenic bird produced from the transgenic blastodermal cells is known as a “founder”. Some founders can be chimeric or mosaic birds if, for example, microinjection does not deliver nucleic acid molecules to all of the blastodermal cells of an embryo. Some founders will carry the transgene in the tubular gland cells in the magnum of their oviducts and will express the heterologous protein encoded by the transgene in their oviducts. If the heterologous protein contains the appropriate signal sequences, it will be secreted into the lumen of the oviduct and onto the yolk of an egg.

Some founders are germline founders. A germline founder is a founder that carries the transgene in genetic material of its germline tissue, and may also carry the transgene in oviduct magnum tubular gland cells that express the heterologous protein. Therefore, in accordance with the invention, the transgenic bird will have tubular gland cells expressing the heterologous protein and the offspring of the transgenic bird will also have oviduct magnum tubular gland cells that express the selected heterologous protein. (Alternatively, the offspring express a phenotype determined by expression of the exogenous gene in a specific tissue of the avian.)

The stably modified oviduct cells will express the heterologous polynucleotide and deposit the resulting polypeptide into the egg white of a laid egg. For this purpose, the expression vector will further comprise an oviduct-specific promoter such as ovalbumin or ovomucoid operably linked to the desired heterologous polynucleotide.

The invention also relates to methods of screening for cells (e.g., avian cells) in which a nucleotide sequence has been inserted. The invention provides for the isolation of such cells by employing the expression of a marker coding sequence. Cells that are contemplated for use as disclosed herein include, without limitation, germline cells which may include sperm cells, ova cells, and embryo cells. The embryos may be for example, stage I, stage II, stage III, stage IV, stage V, stage VI, stage VII, stage VIII, stage IX, stage X, stage XI or stage XII embryos. In one particularly useful embodiment, the cells contemplated for use include blastodermal cells.

In one embodiment, a first nucleotide sequence comprising a first recombination site, such as recombination sites disclosed elsewhere herein (e.g., an attP site), also includes a functional transcription initiation site. Any useful functional transcription initiation site may be employed. In one embodiment, a U3 promoter is employed. In one embodiment, a long terminal repeat (LTR) region of a retrovirus is employed as the transcription initiation site. For example, a LTR which includes a U3 promoter may be employed.

Examples of other useful transcription initiation sites may include, without limitation, Pol III promoters (including type 1, type 2 and type 3 Pol III promoters) such as H1 promoters, U6 promoters, tRNA promoters, RNase MPR promoters and functional portions of each of these promoters. Other promoters that may be useful in the present invention include, without limitation, Pol I promoters, Pol II promoters, cytomegalovirus (CMV) promoters, rous-sarcoma virus (RSV) promoters, avian leukemia virus (ALV) promoters, actin promoters such as beta actin promoters, murine leukemia virus (MLV) promoters, mouse mammary tumor virus (MMTV) promoters, SV40 promoters, ovalbumin promoters, lysozyme promoters, conalbumin promoters, ovomucoid promoters, ovomucin promoters, ovotransferrin promoters and functional portions of each of these promoters.

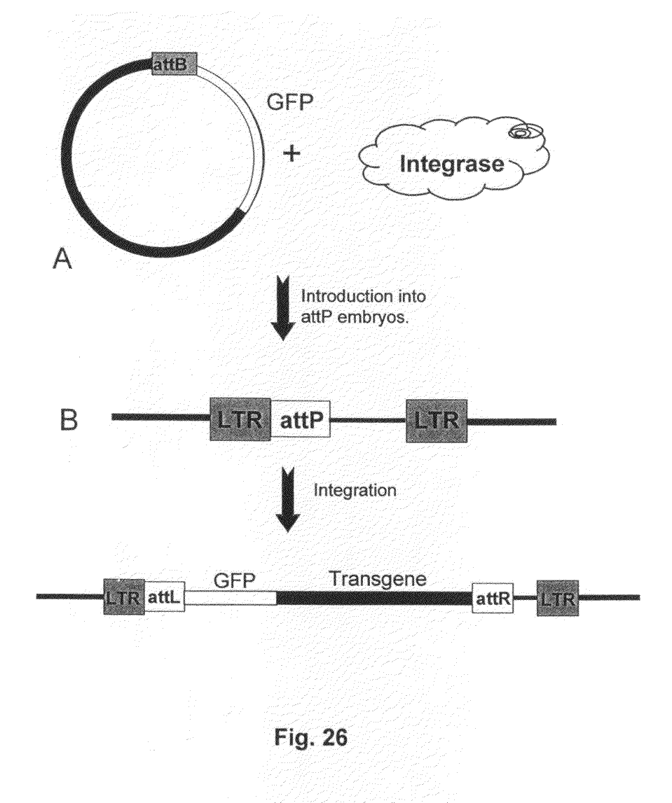

In accordance with the present methods, the first nucleotide sequence comprising the first recombination site and transcription initiation site is inserted into a genome of a cell by any useful method. For example, the first nucleotide sequence may be inserted into the genome as part of a retrovirus construct (e.g., ALV). For example, a retrovirus comprising an attP site may be transduced into the genome of the cell (FIG. 26).

The invention provides for the introduction of a second nucleotide sequence, which includes a second recombination site such as recombination sites disclosed elsewhere herein (e.g., an attB site) a nucleotide sequence of interest (denote as “transgene” in FIG. 26) and a promoterless marker coding sequence, into one or more cells which include the first nucleotide sequence in their genome.

Any useful method for the introduction of the nucleotide sequences into the cells is contemplated for use herein. Exemplary delivery systems for the nucleic acids include, without limitation, liposomal derived systems, poly-lysine conjugates, protoplast fusion, microinjection and electroporation.

Any useful marker coding sequence may be employed in the present screening methods. For example, a bioluminescent protein coding sequence may serve as the marker coding sequence for use as disclosed herein. In one embodiment, the present invention contemplates the use of a green fluorescent protein (GFP) marker gene coding sequence. In one embodiment, antibiotic resistance is the marker.

In one embodiment, the marker coding sequence is positioned such that when integration occurs between the first and second recombination sites, the marker expression will be under the control of the transcription initiation site of the first nucleotide sequence and will be expressed. Cells in which integration has occurred can be identified by expression of the marker coding sequence.

The present invention provides for the isolation of one or more cells in which the marker coding sequence is expressed. In the case of bioluminescent markers such as GFP, the cells may be sorted and thereafter isolated using flow cytometry by methods well known in the art such as those methods disclosed in de Jong et al. Cytometry 35: 129-133 (1999) and Griffin et al. Cytogenet. Cell Genet. 87: 278-281 (1999). Any useful methods of cell separation or isolation are contemplated for use herein including mechanical isolation or the use of laser scissors and tweezers, and the like.

In one useful embodiment, the second nucleotide sequence is introduced into blastodermal cells which include the first nucleotide sequence in their genome. For example, the blastodermal cells may comprise avian blastodermal cells isolated from fertile embryos, such as stage VII to stage XII embryos. Blastodermal cells in which the marker coding sequence is expressed are isolated and introduced into the subgerminal cavity of fertile eggs. Suitable methods for the manipulation of avian eggs, including opening and resealing hard shell eggs are described in U.S. Pat. Nos. 5,897,998 and 6,397,777 the disclosures of which are incorporated herein by reference in their entireties. The eggs are hatched and the chicks raised to maturity by methods well known in the field.

This description uses gene nomenclature accepted by the Cucurbit Genetics Cooperative as it appears in the Cucurbit Genetics Cooperative Report 18:85 (1995), which are incorporated herein by reference in its entirety.

The disclosures of publications such as journal articles, patents, and published patent applications referred to in this application are hereby incorporated by reference in their entirety into the present application.

It will be apparent to those skilled in the art that various modifications, combinations, additions, deletions and variations can be made in the present invention without departing from the scope or spirit of the invention. For instance, features illustrated or described as part of one embodiment can be used in another embodiment to yield a still further embodiment. It is intended that the present invention covers such modifications, combinations, additions, deletions and variations as come within the scope of the appended claims and their equivalents.

The present invention is further illustrated by the following examples, which are provided by way of illustration and should not be construed as limiting. The contents of all references, published patents and patents cited throughout the present application are hereby incorporated by reference in their entireties.

### Example 1

### Phage phiC31 Integrase Functions in Avian Cells

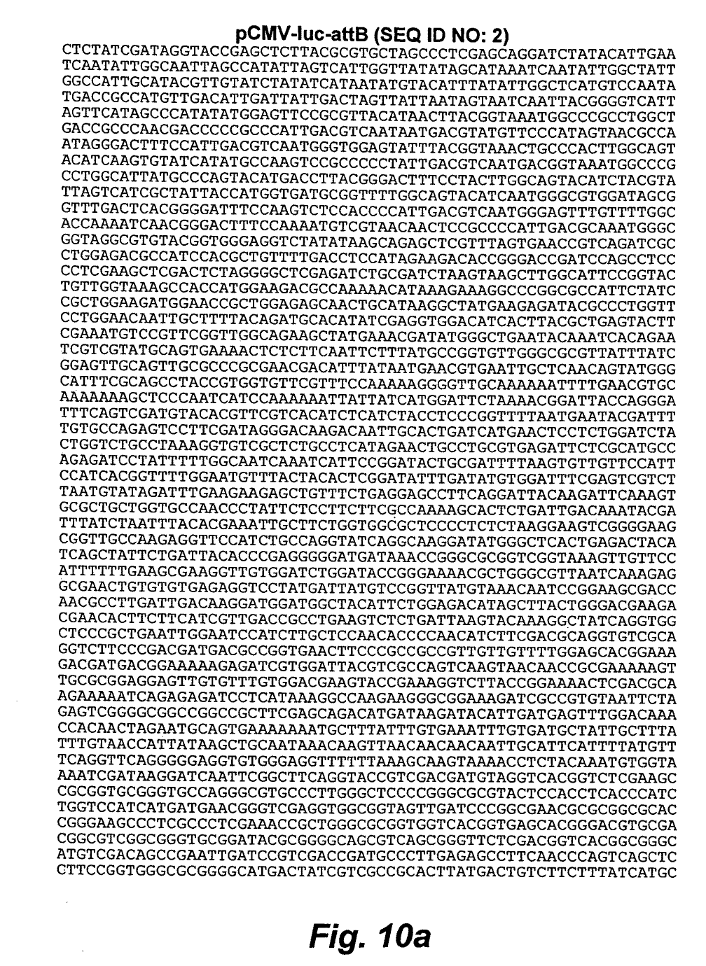

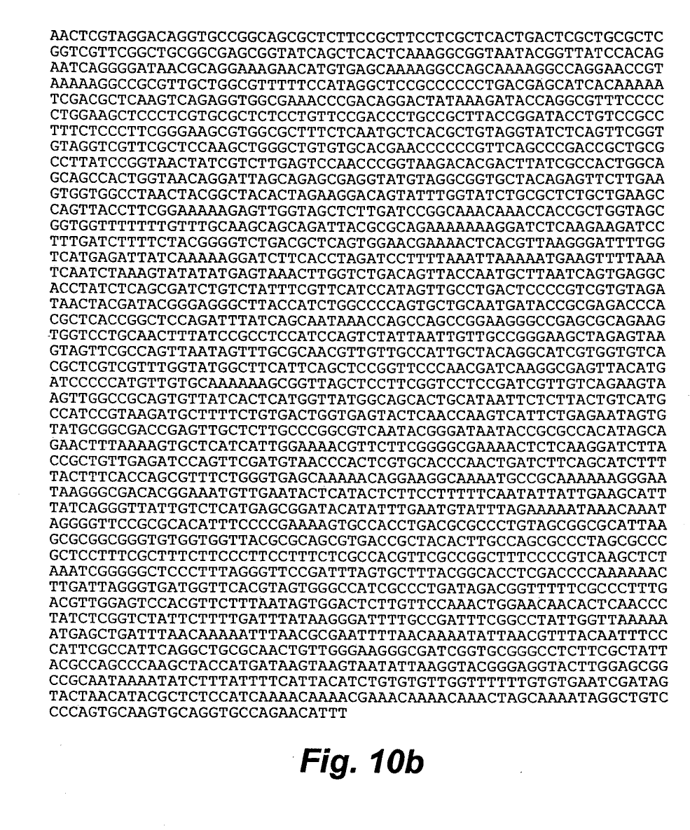

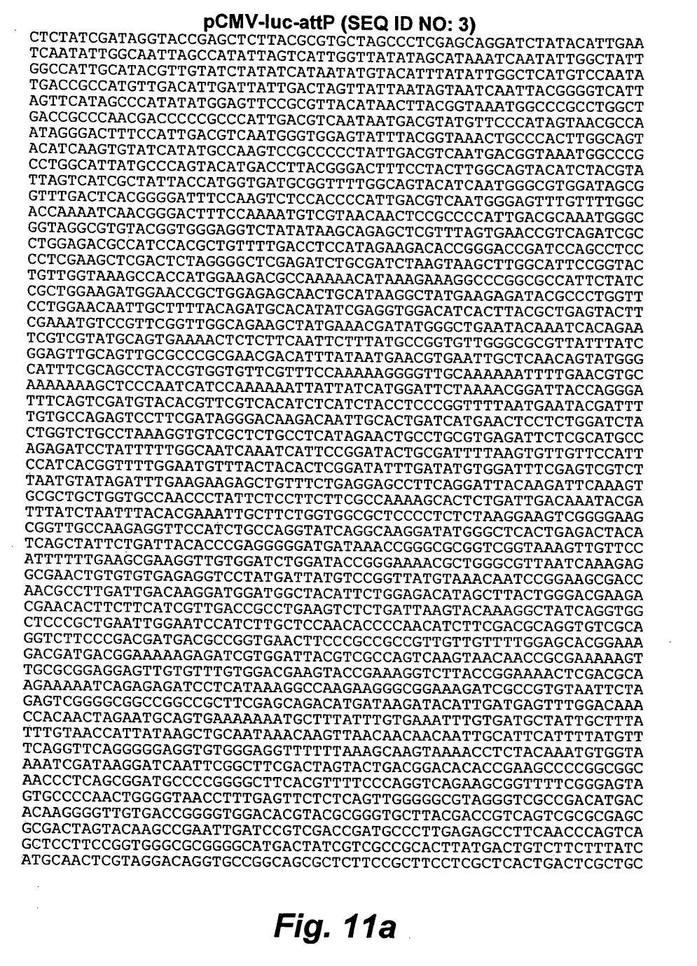

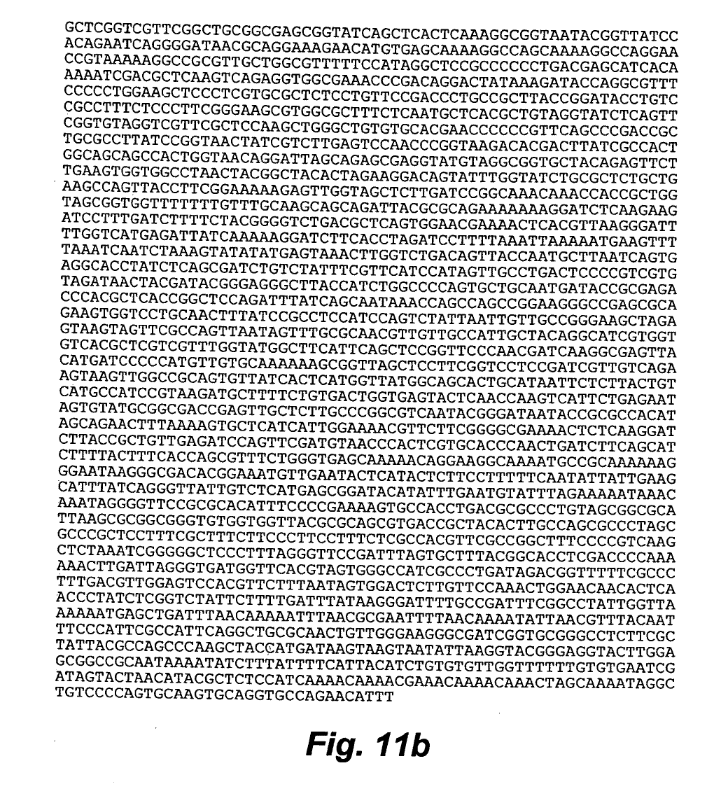

(a) A luciferase vector bearing either an attB (SEQ ID NO: 2 shown in FIG. 10) or attP (SEQ ID NO: 3 shown in FIG. 11) site was cotransfected with an integrase expression vector CMV-C31int (SEQ ID NO: 1) into DF-1 cells, a chicken fibroblast cell line. The cells were passaged several times and the luciferase levels were assayed at each passage.

Cells were passaged every 3-4 days and one third of the cells were harvested and assayed for luciferase. The expression of luciferase was plotted as a percentage of the expression measured 4 days after transfection. A luciferase expression vector bearing an attP site as a control was also included.

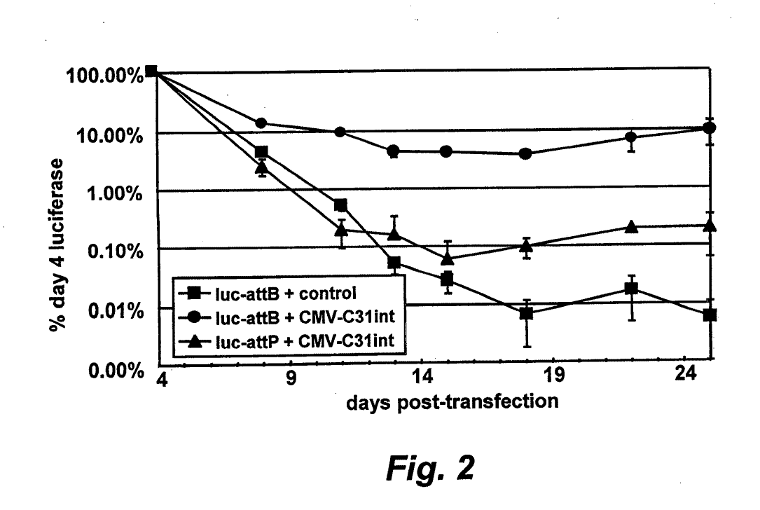

As can be seen in FIG. 2, in the absence of integrase, luciferase expression from a vector bearing attP or attB decreased to very low levels after several days. However, luciferase levels were persistent when the luciferase vector bearing attB was cotransfected with the integrase expression vector, indicating that the luciferase vector had stably integrated into the avian genome.

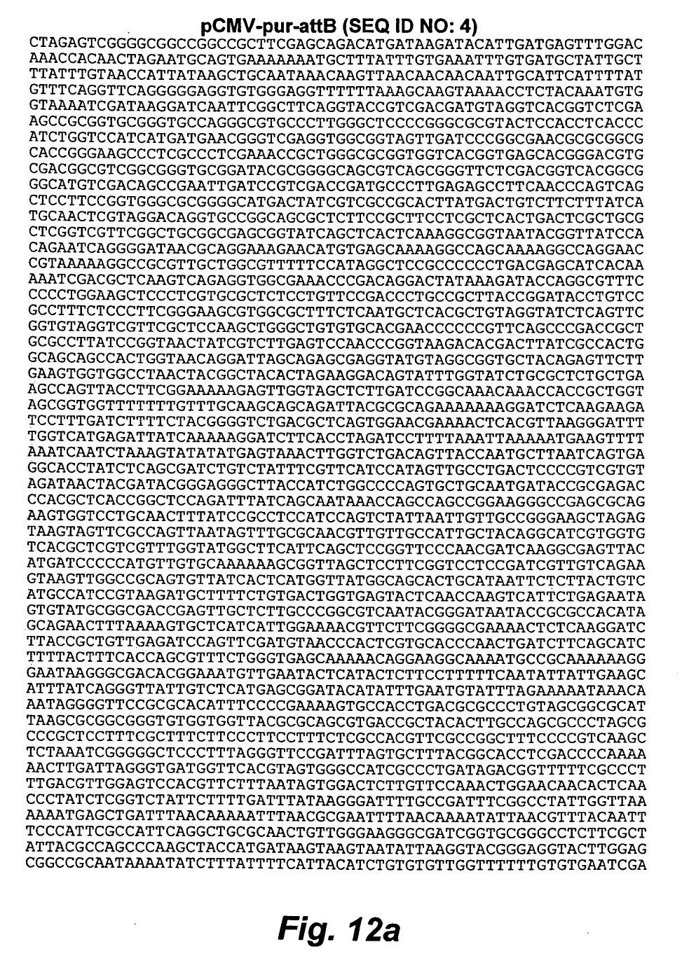

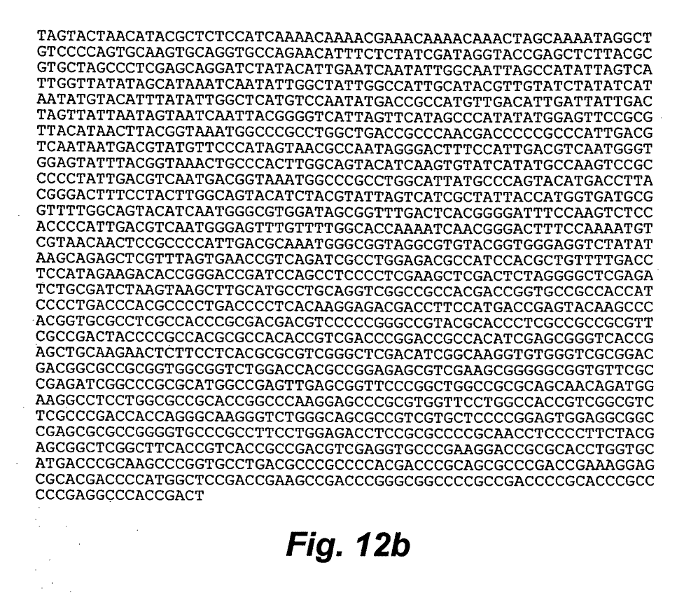

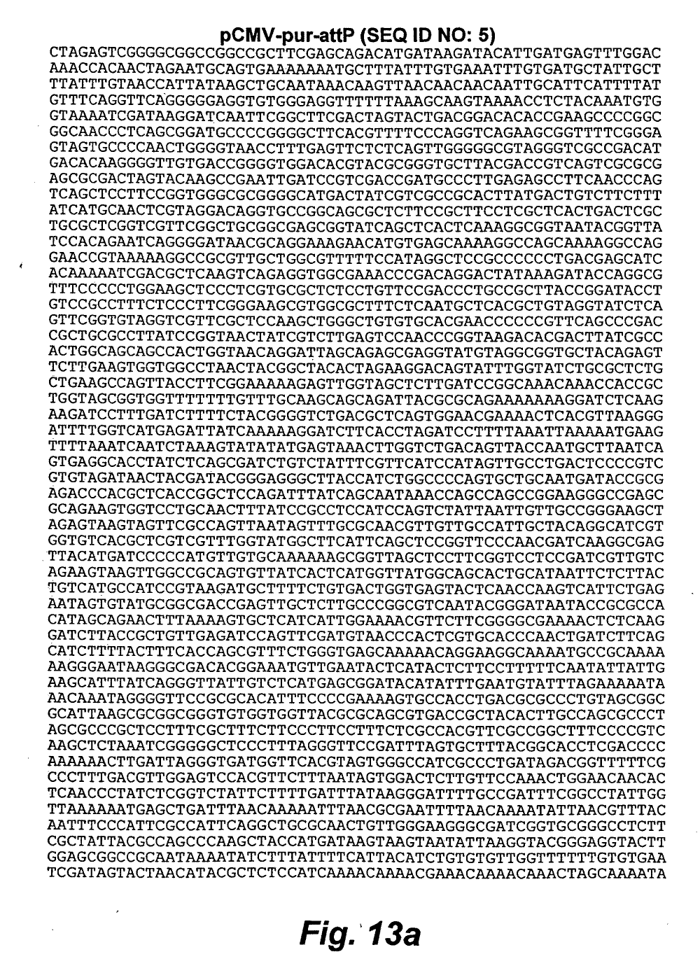

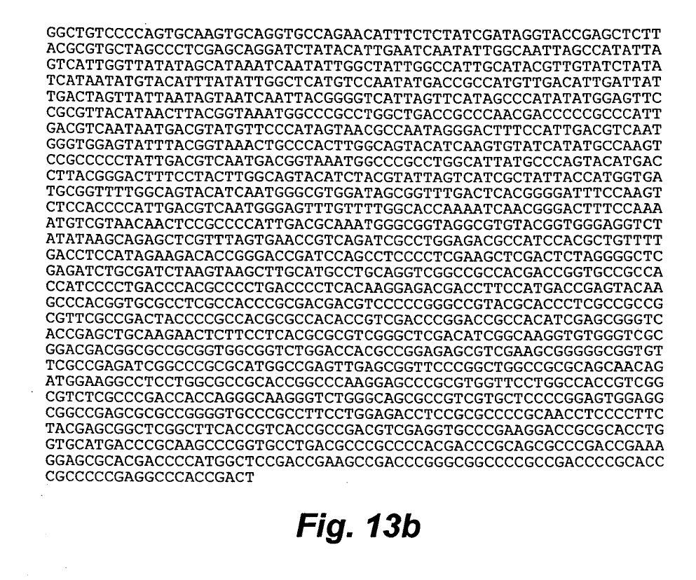

(b) A drug-resistance colony formation assay was used to quantitate integration efficiency. The puromycin resistance expression vector pCMV-pur was outfitted with an attB (SEQ ID NO: 4 shown in FIG. 12) or an attP (SEQ ID NO: 5 shown in FIG. 13) sites. Puromycin resistance vectors bearing attB sites were cotransfected with phiC31 integrase or a control vector into DF-1 cells. One day after transfection, puromycin was added. Puromycin resistant colonies were counted 12 days post-transfection.

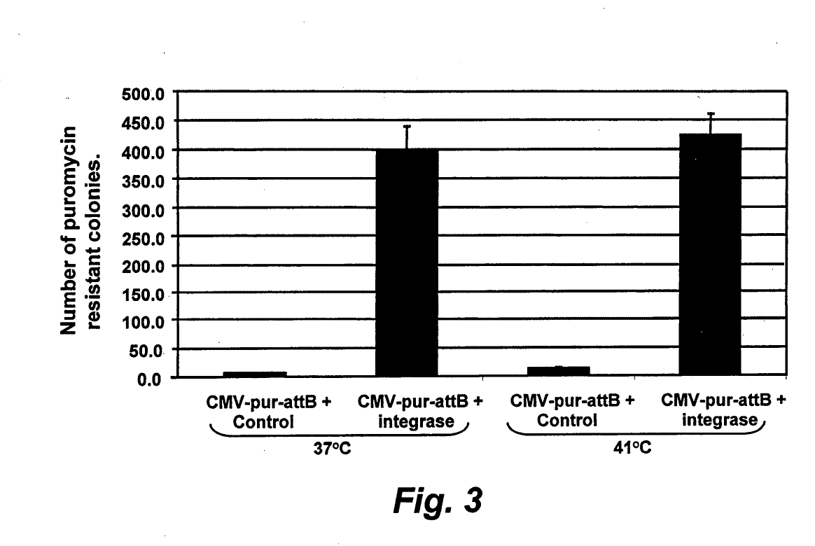

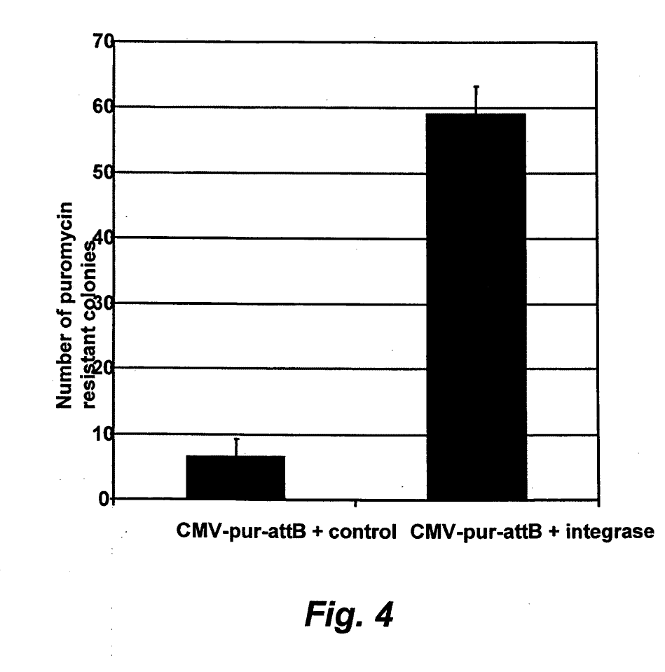

In the absence of cotransfected integrase expression, few DF-1 cell colonies were observed after survival selection. When integrase was co-expressed, multiple DF-1 cell colonies were observed, as shown in FIG. 3. Similar to the luciferase expression experiment, the attB sequence (but not the attP sequence) was able to facilitate integration of the plasmid into the genome. FIG. 3 also shows that phiC31 integrase functions at both 37° Celsius and 41° Celsius. Integrase also functions in quail cells using the puromycin resistance assay, as shown in FIG. 4.

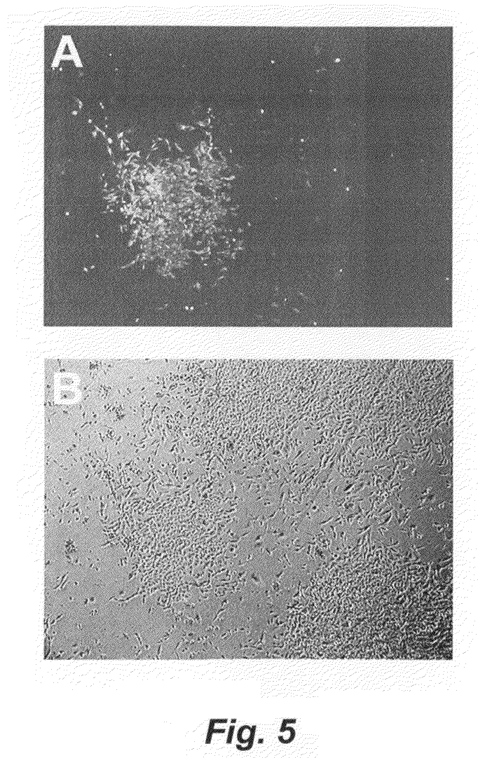

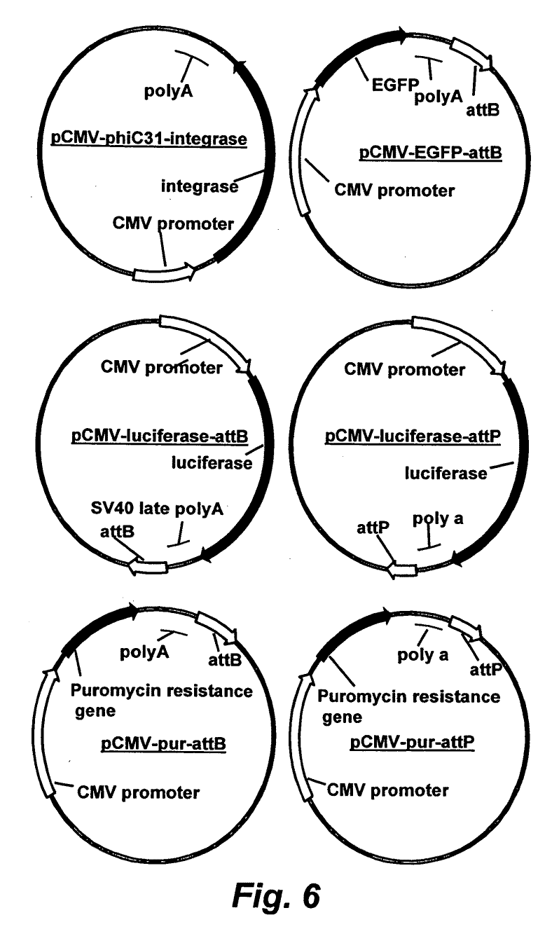

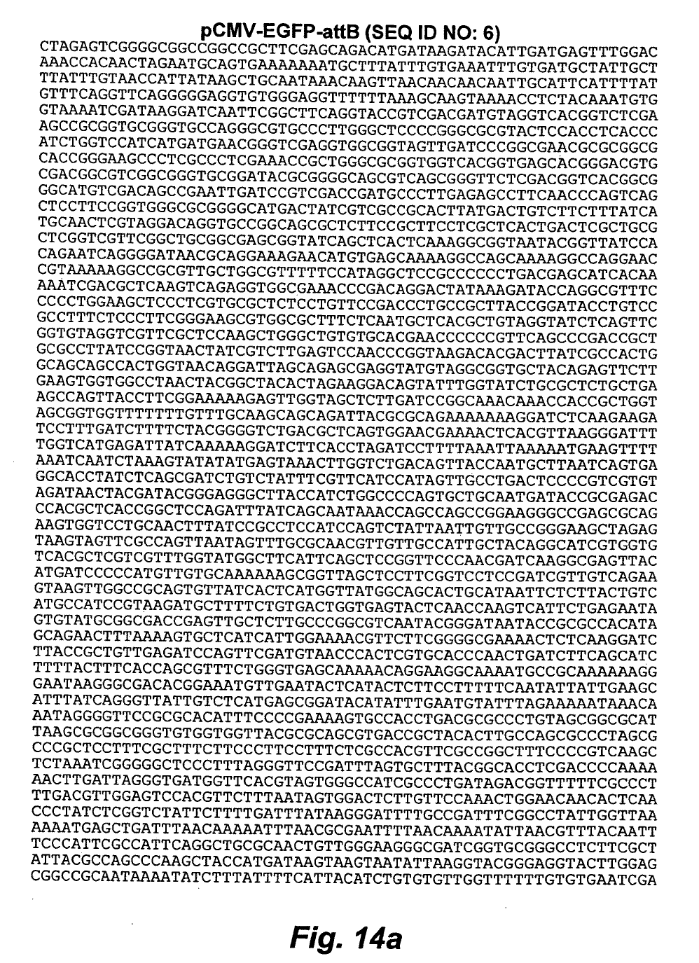

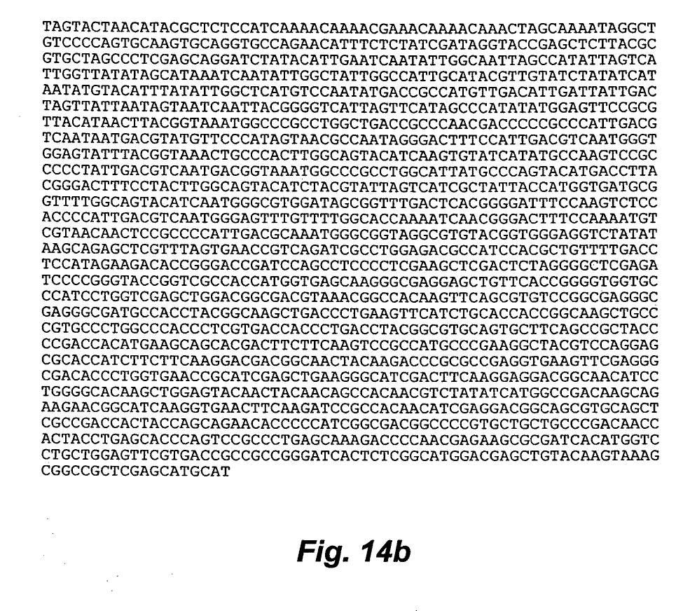

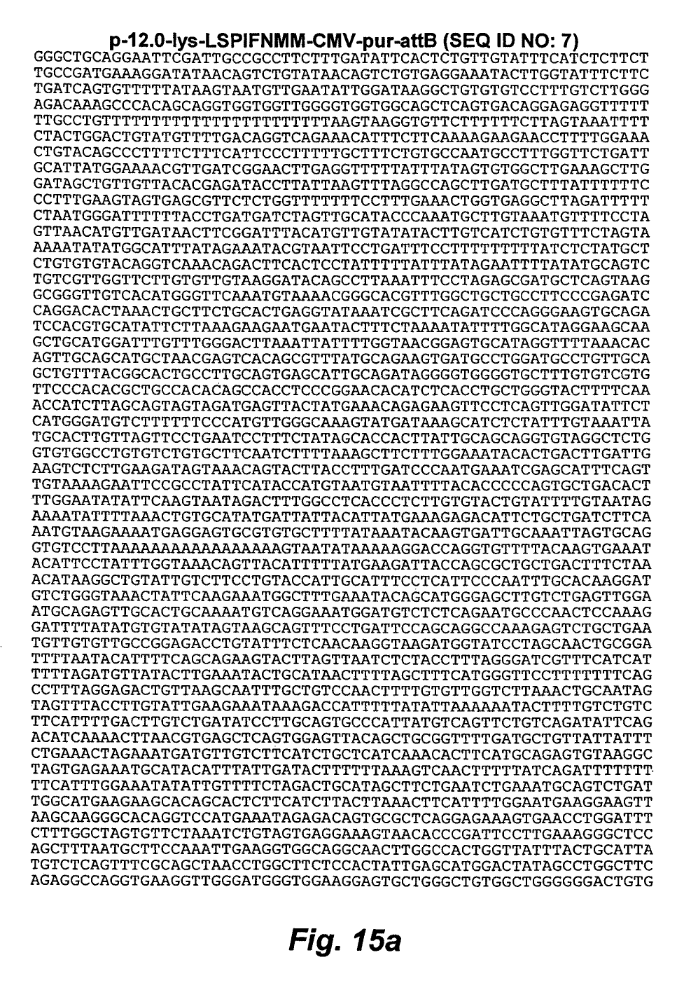

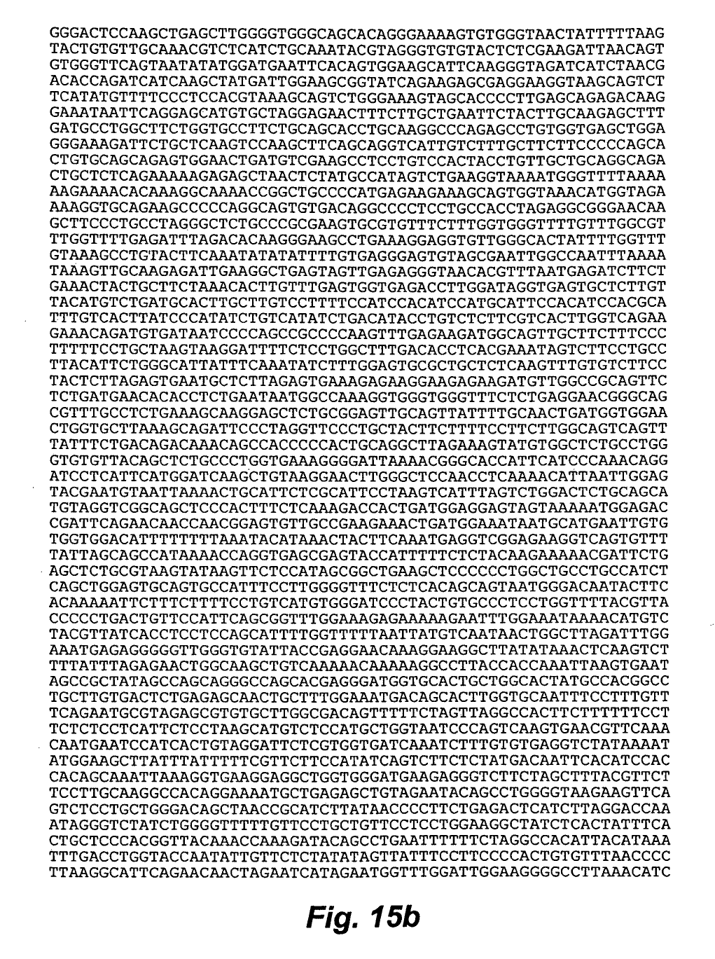

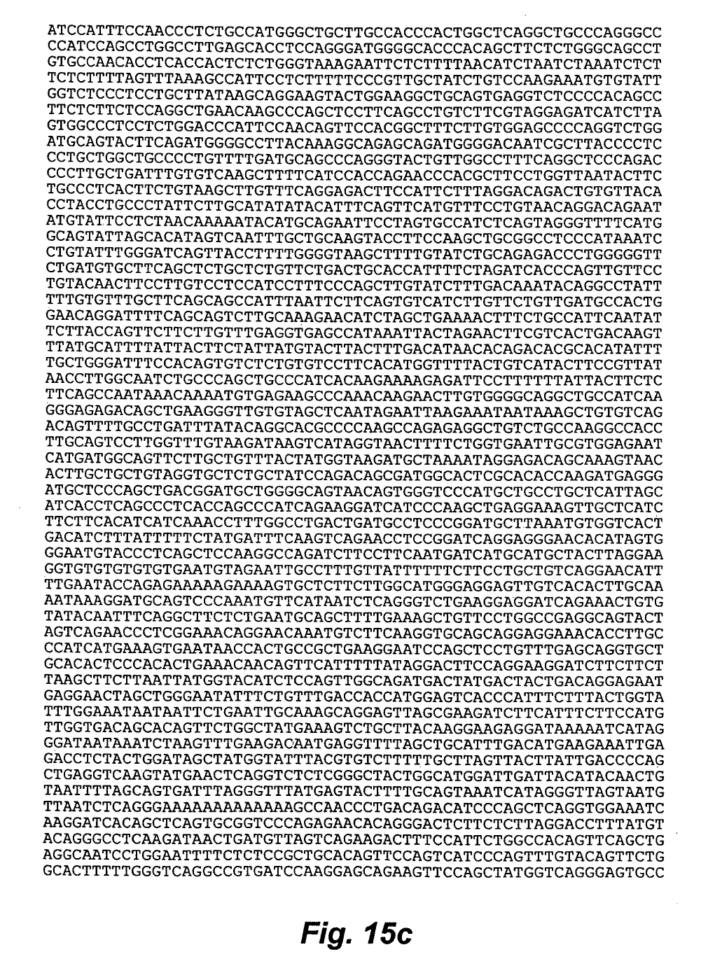

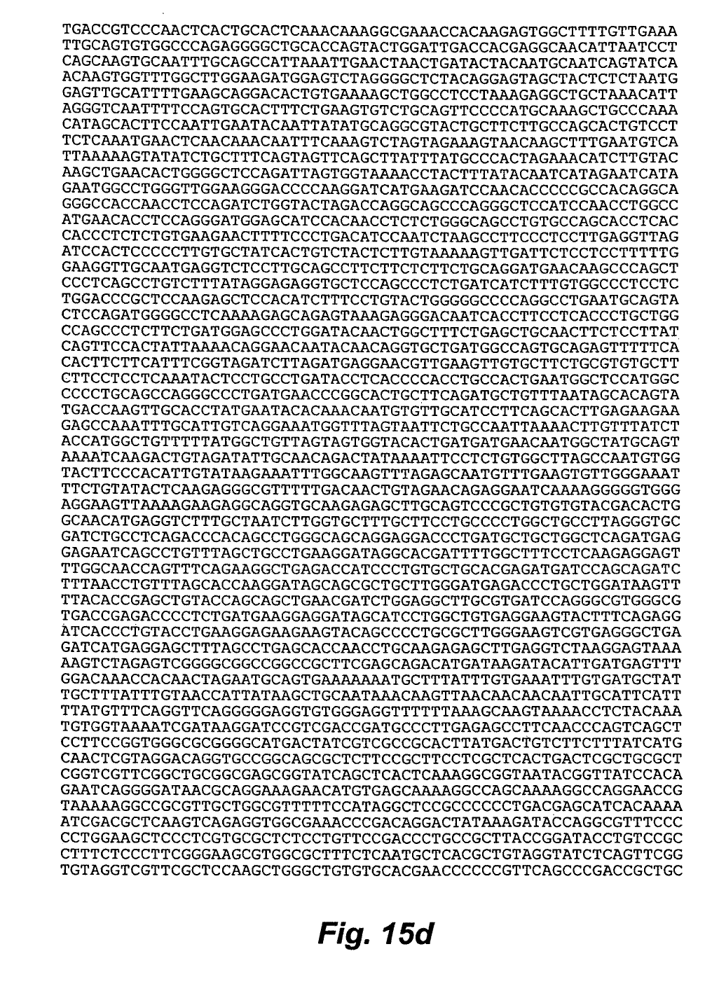

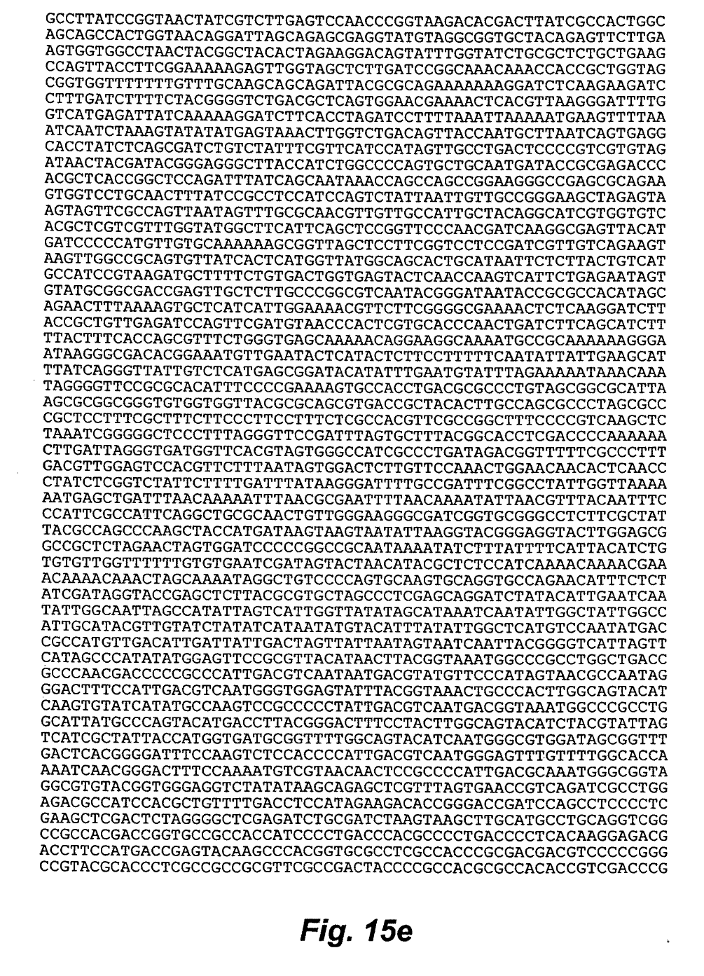

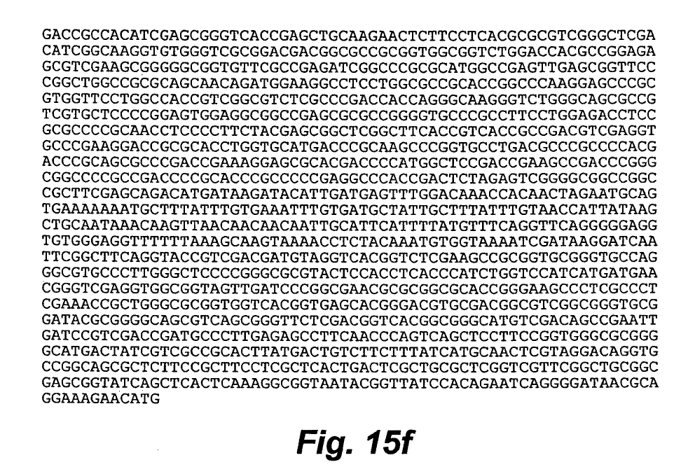

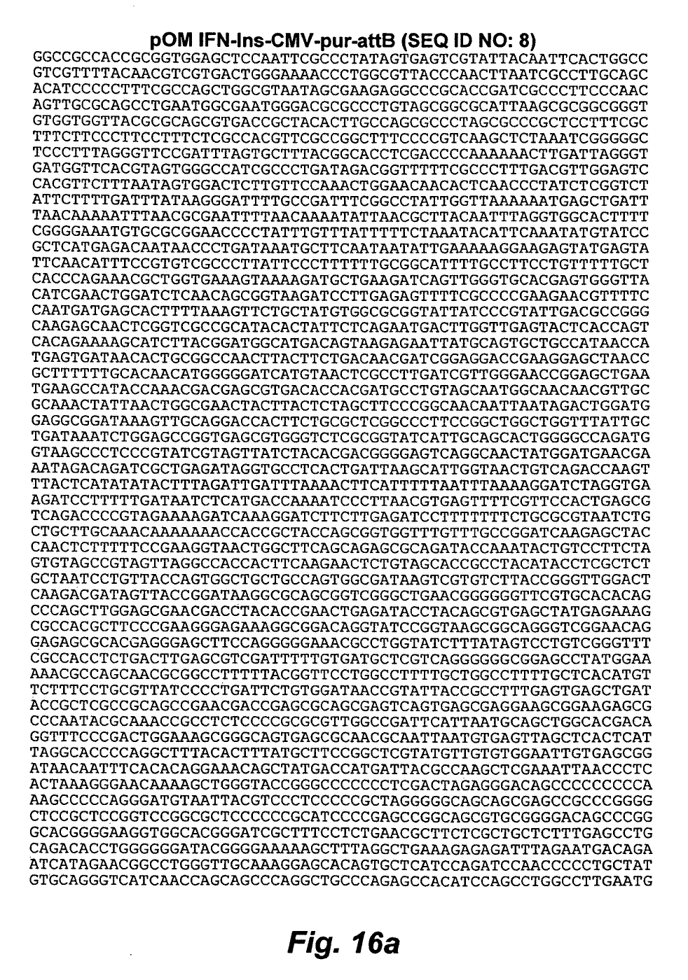

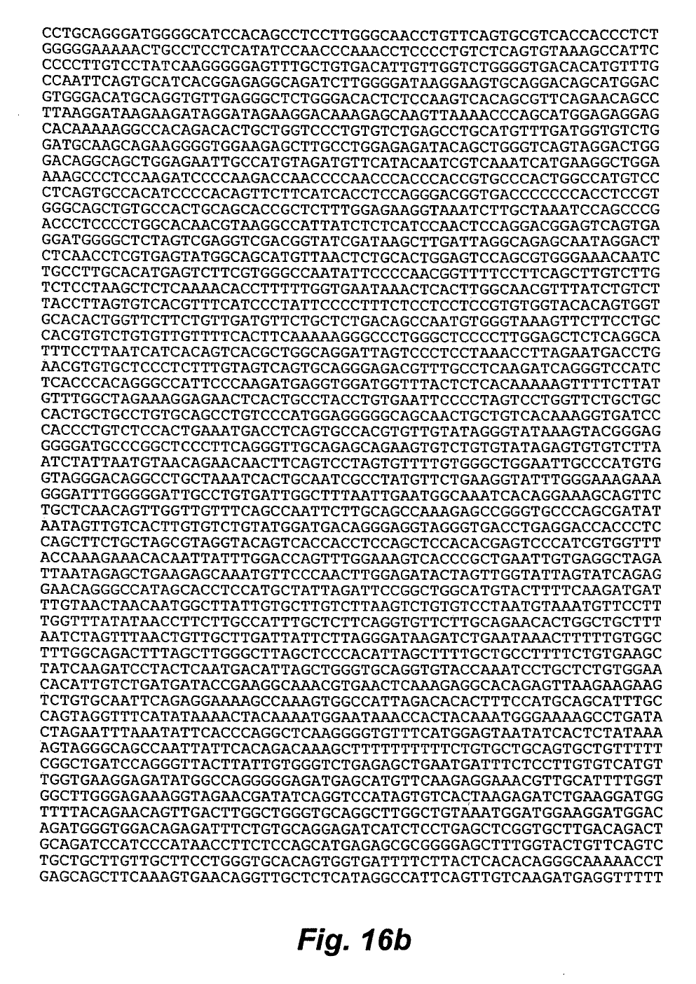

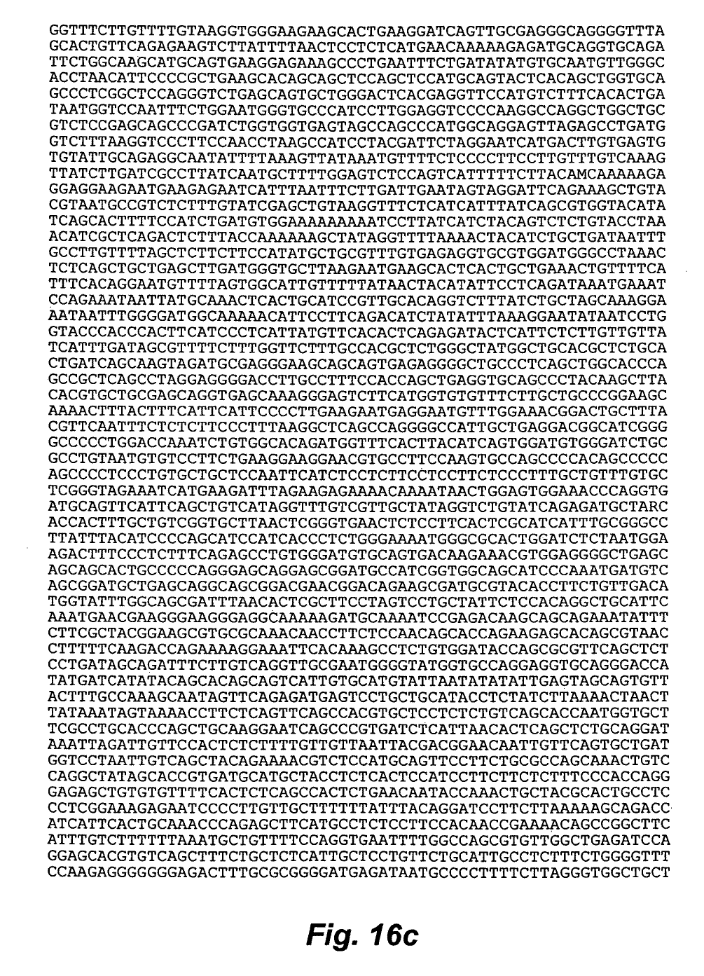

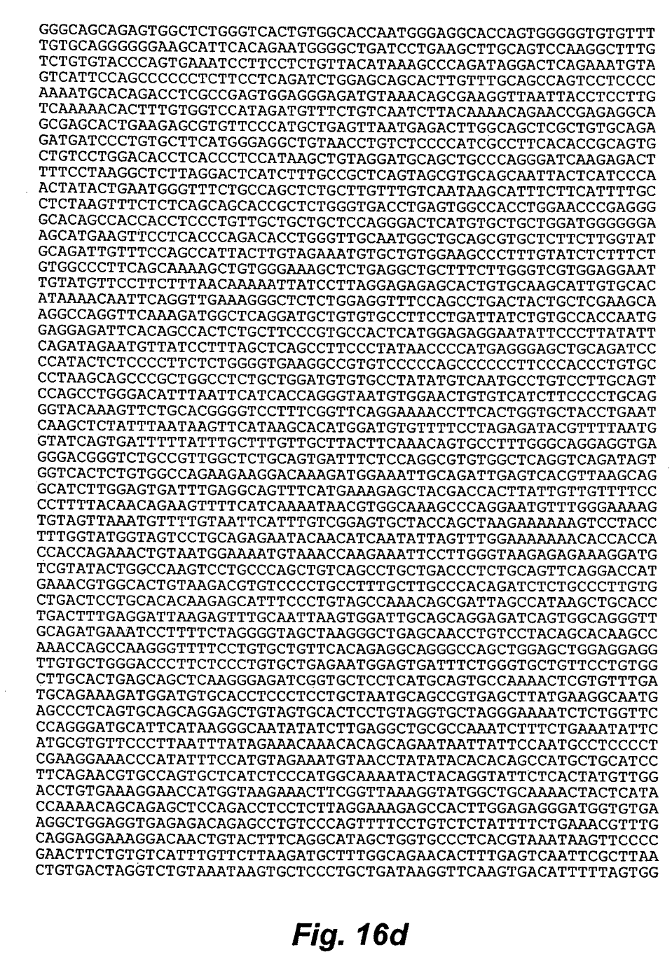

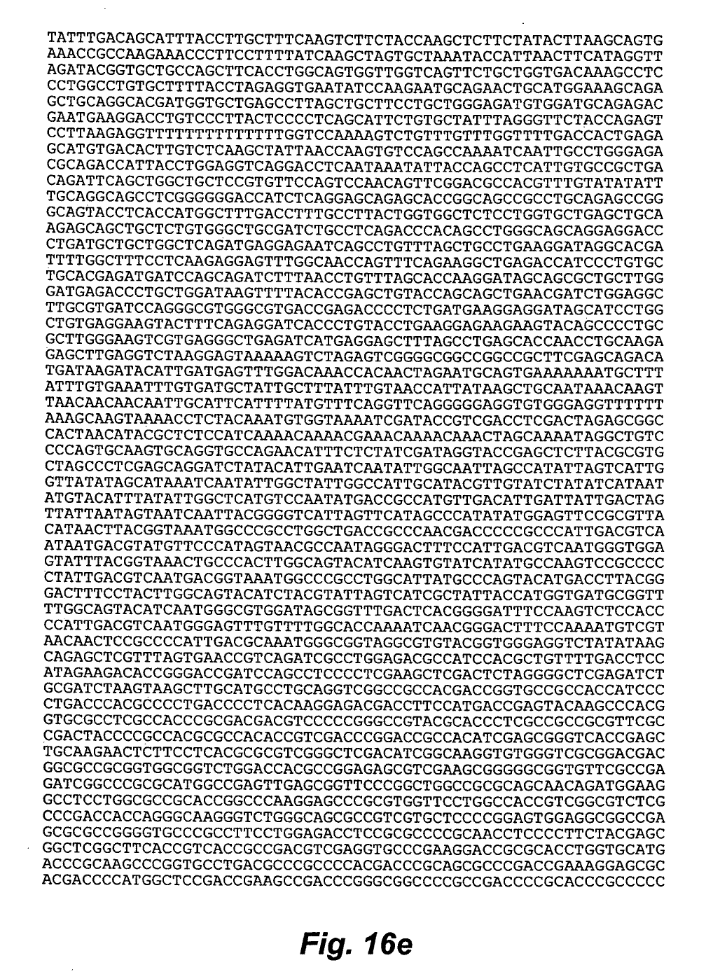

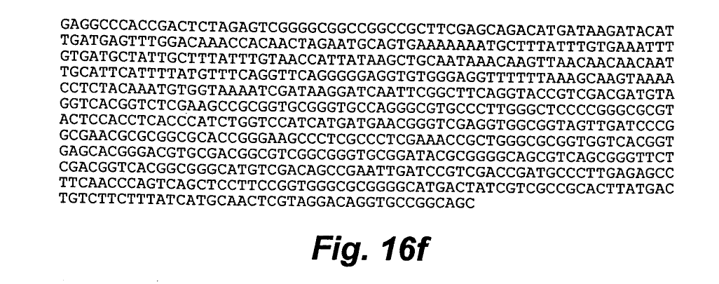

(c) The CMV-pur-attB vector (SEQ ID NO: 4) was also cotransfected with an enhanced green fluorescent protein (EGFP) expression vector bearing an attB site (SEQ ID NO: 6 shown in FIG. 14) into DF-1 cells and the phiC31 integrase expression vector CMV-C31int (SEQ ID NO: 1). After puromycin selection for 12 days, the colonies were viewed with UV light to determine the percentage of cells that expressed EGFP. Approximately 20% of puromycin resistant colonies expressed EGFP in all of the cells of the colony, as shown in FIG. 5, indicating that the integrase can mediate multiple integrations per cell.
(d) PhiC31 integrase promoted the integration of large transgenes into avian cells. A puromycin expression cassette comprising a CMV promoter, puromycin resistance gene, polyadenylation sequence and the attB sequence was inserted into a vector containing a 12.0 kb lysozyme promoter and the human interferon α2b gene (SEQ ID NO: 7 shown in FIG. 15) and into a vector containing a 10.0 kb ovomucoid promoter and the human interferon α2b gene (SEQ ID NO: 8) as shown in FIG. 16.

DF-1 cells were transfected with donor plasmids of varying lengths bearing a puromycin resistance gene and an attB sequence in the absence or presence of an integrase expression plasmid. Puromycin was added to the culture media to kill those cells which did not contain a stably integrated copy of the puromycin resistance gene. Cells with an integrated gene formed colonies in the presence of puromycin in 7-12 days. The colonies were visualized by staining with methylene blue and the entire 60 mm culture dish was imaged.

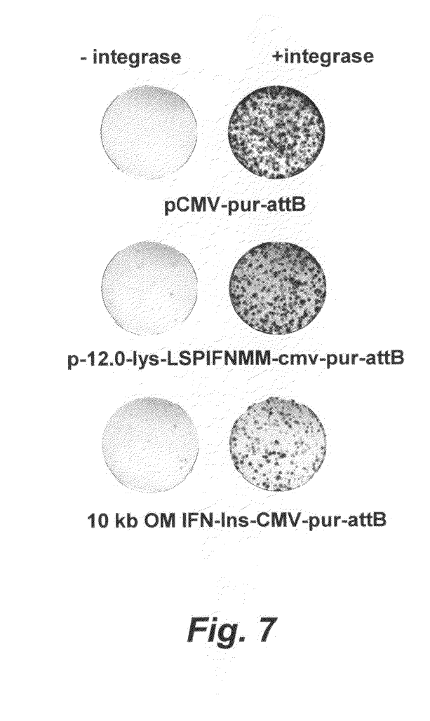

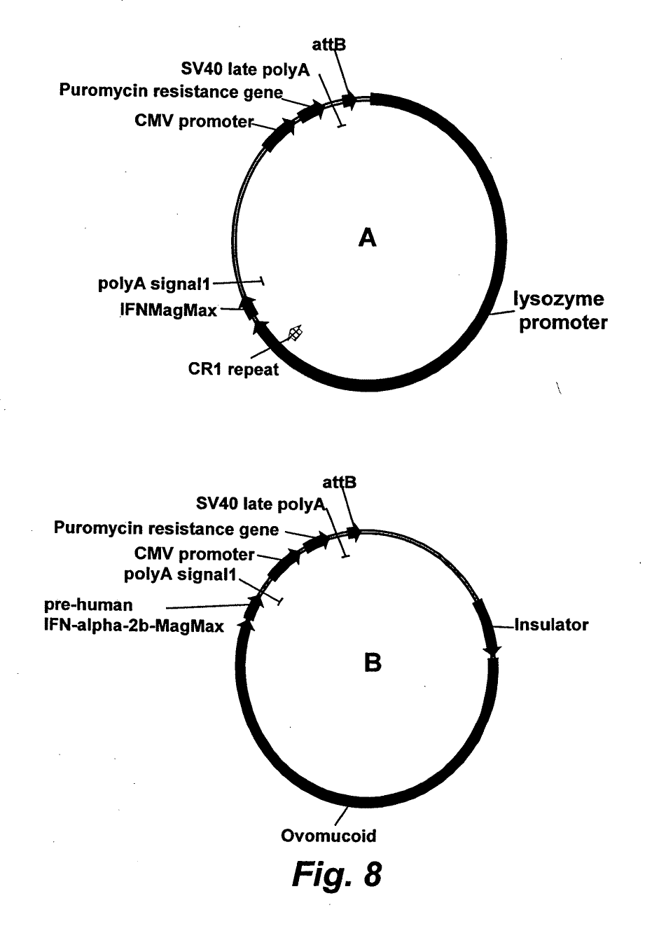

PhiC31 integrase mediated the efficient integration of both vectors as shown in FIG. 7.

### Example 2

### Cell Culture Methods

DF-1 cells were cultured in DMEM with high glucose, 10% fetal bovine serum, 2 mM L-glutamine, 100 units/ml penicillin and 100 μg/ml streptomycin at 37° Celsius and 5% CO₂. A separate population of DF-1 cells was grown at 41° Celsius. These cells were adapted to the higher temperature for one week before they were used for experiments.

Quail QT6 cells were cultured in F10 medium (Gibco) with 5% newborn calf serum, 1% chicken serum heat inactivated (at 55° Celsius for 45 mins), 10 units/ml penicillin and 10 μg/ml streptomycin at 37° Celsius and 5% CO₂.

### Example 3

### Selection and Assay Methods

(a) Puromycin selection assay: About 0.8×10⁶ DF-1 (chicken) or QT6 (quail) cells were plated in 60 mm dishes. The next day, the cells were transfected as follows:

10 to 50 ng of a donor plasmid and 1 to 10 μg of an Integrase-expressing plasmid DNA were mixed with 150 μl of OptiMEM. 15 μl of DMRIE-C was mixed with 150 μl of OptiMEM in a separate tube, and the mixtures combined and incubated for 15 mins. at room temperature.

While the liposome/DNA complexes were forming, the cells were washed with OptiMEM and 2.5 ml of OptiMEM was added. After 15 minutes, 300 μl of the DNA-lipid mixture was added drop wise to the 2.5 ml of OptiMEM covering the cell layers. The cells were incubated for 4-5 hours at either 37° Celsius or 41° Celsius, 5% CO₂. The transfection mix was replaced with 3 mls of culture media. The next day, puromycin was added to the media at a final concentration of 1 μg/ml, and the media replaced every 2 to 4 days. Puromycin resistant colonies were counted or imaged 10-12 days after the addition of puromycin.

(b) Luciferase assay: Chicken DF-1 or quail QT6 cells (0.8×10⁶) were plated in 60 mm dishes. Cells were transfected as described above. The cells from a plate were transferred to a new 100 mm plate when the plate became confluent, typically on day 3-4, and re-passaged every 3-4 days.

At each time point, one-third of the cells from a plate were replated, and one-third were harvested for the luciferase assay. The cells were pelleted in an eppendorf tube and frozen at −70° C.

The cell pellet was lysed in 200 μl of lysis buffer (25 mM Tris-acetate, pH7.8, 2 mM EDTA, 0.5% Triton X-100, 5% glycerol). Sample (5 μl) was assayed using the Promega BrightGlo reagent system.

(c) Visualization of EGFP: EGFP expression was visualized with an inverted microscope with FITC illumination [Olympus IX70, 100 W mercury lamp, HQ-FITC Band Pass Emission filter cube, exciter 480/40 nm, emission 535/50 nm, 20× phase contrast objective (total magnification was 2.5×10×20)].
(d) Staining of cell colonies: After colonies had formed, typically after 7-12 days of culture in puromycin medium, the cells were fixed in 2% formaldehyde, 0.2% glutaraldehyde for 15 mins, and stained in 0.2% methylene blue for 30 mins. followed by several washes with water. The plates were imaged using a standard CCD camera in visible light.

### Example 4

### Production of Genetically Transformed Avian Cells

Avian stage X blastodermal cells are used as the cellular vector for the transgenes. Stage X embryos are collected and the cells dispersed and mixed with plasmid DNA. The transgenes are then introduced to blastodermal cells via electroporation. The cells are immediately injected back into recipient embryos.

The cells are not cultured for any time period to ensure that they remain capable of contributing to the germline of resulting chimeric embryos. However, because there is no culture step, cells that bear the transgene cannot be identified. Typically, only a small percentage of cells introduced to an embryo will bear a stably integrated transgene (0.01 to 1%). To increase the percentage of cells bearing a transgene, therefore, the transgene vector bears an attB site and is co-electroporated with a vector bearing the CMV promoter driving expression of the phiC31 transgene (CMV-C31int (SEQ ID NO: 1). The integrase then drives integration of the transgene vector into the nuclear genome of the avian cell and increases the percentage of cells bearing a stable transgene.

(a) Preparation of avian stage X blastodermal cells:
i) Collect fertilized eggs from Barred Rock or White leghorn chickens (Gallus gallus) or quail (Japonica coturnix) within 48 hrs. of laying;
ii) Use 70% ethanol to clean the shells;
iii) Crack the shells and open the eggs;
iv) Remove egg whites by transferring yolks to opposite halves of shells, repeating to remove most of the egg whites;
v) Put egg yolks with embryo discs facing up into a 10 cm petri dish;
vi) Use an absorbent tissue to gently remove egg white from the embryo discs;
vii) Place a Whatman filter paper 1 ring over the embryos;
viii) Use scissors to cut the membranes along the outside edge of the paper ring while gently lifting the ring/embryos with a pair of tweezers;
ix) Insert the paper ring with the embryos at a 45 degree angle into a petri dish containing PBS-G solution at room temperature;
x) After ten embryo discs are collected, gently wash the yolks from the blastoderm discs using a Pasteur pipette under a stereo microscope;
xi) Cut the discs by a hair ring cutter (a short piece of human hair is bent into a small loop and fastened to the narrow end of a Pasteur pipette with Parafilm);
xii) Transfer the discs to a 15 ml sterile centrifuge tube on ice;
xiii) Place 10 to 15 embryos per tube and allow to settle to the bottom (about 5 mins.);
xiv) Aspirate the supernatant from the tube;
xv) Add 5 mls of ice-cold PBS without Ca⁺⁺ and Mg⁺⁺, and gently pipette 4 to 5 times using a 5 mls pipette;
xvi) Incubate in ice for 5-7 mins. to allow the blastoderms to settle, and aspirate the supernatant;
xvii) Add 3 mls of ice cold 0.05% trypsin/0.02% ETDA to each tube and gently pipette 3 to 5 times using a 5 ml pipette;
xviii) Put the tube in ice for 5 mins. and then flick the tube by finger 40 times. Repeat;
xix) Add 0.5 mls FBS and 3-5 mls BDC medium to each tube and gently pipette 5-7 times using a 5 ml pipette;
xx) Spin at 500 rpm (RCF 57×g) at 4° Celsius for 5 mins;
xxi) Remove the supernatant and add 2 mls ice cold BDC medium into each tube; and
xxii) Resuspend the cells by gently pipetting 20-25 times; and
xxiii) Determine the cell titer by hemacytometer and ensure that about 95% of all BDCs are single cells, and not clumped.
   (b) Transfection of linearized plasmids into blastodermal cells by small scale electroporation:
i) Centrifuge the blastodermal cell suspension from step (xxiii) above at RCF 57×g, 4° Celsius, for 5 mins;
ii) Resuspend cells to a density of 1-3×10⁶ per ml with PBS without Ca²⁺ and Mg²⁺.
iii) Add linearized DNA, 1-30 μg per 1-3×10⁵ blastodermal cells in an eppendorf tube at room temperature. Add equimolar molar amounts of the non-linearized transgene plasmid bearing an, attB site, and an integrase expression plasmid;
iv) Incubate at room temperature for 10 mins;
v) Aliquot 100 μl of the DNA-cell mixture to a 0.1 cm cuvette at room temperature;
vi) Electroporate at 240 V and 25 μFD (or 100 V and 125 μFD for quail cells) using, for example, a Gene Pulser II™ (BIO-RAD).
vii) Incubate the cuvette at room temperature for 1-10 mins.
viii) Before the electroporated cells are injected into a recipient embryo, they are transferred to a eppendorf tube at room temperature. The cuvette is washed with 350 μl of media, which is transferred to the eppendorf, spun at room temperature and re-suspended in 0.01-0.3 ml medium;
ix) Inject 1-10 μl of cell suspension into the subgerminal cavity of an non-irradiated or, for example, an irradiated (e.g., with 300-900 rads) stage X egg. Shell and shell membrane are removed and, after injection, resealed according to U.S. Pat. No. 6,397,777, issued Jun. 6, 2002, the disclosure of which is incorporated herein by reference in its entirety; and
x) The egg is then incubated to hatching.

### (c) Blastodermal Cell Culture Medium:

i) 409.5 mls DMEM with high glucose, L-glutamine, sodium pyruvate, pyridoxine hydrochloride;
ii) 5 mls Men non-essential amino acids solution, 10 mM;
iii) 5 mls Penicillin-streptomycin 5000 U/ml each;
iv) 5 mls L-glutamine, 200 mM;
v) 75 mls fetal bovine serum; and
vi) 0.5 mls β-mercaptoethanol, 11.2 mM.

### Example 5

### Transfection of Stage X Embryos with attB Plasmids

(a) DNA-PEI: Twenty-five μg of a phage phiC31 integrase expression plasmid (pCMV-int), and 25 μg of a luciferase-expressing plasmid (pβ-actin-GFP-attB) are combined in 200 μl of 28 mM Hepes (pH 7.4). The DNA/Hepes is mixed with an equal volume of PEI which has been diluted 10-fold with water. The DNA/Hepes/PEI is incubated at room temperature for 15 mins Three to seven μl of the complex are injected into the subgerminal cavity of windowed stage X white leghorn eggs which are then sealed and incubated as described in U.S. Pat. No. 6,397,777, issued Jun. 6, 2002. The complexes will also be incubated with blastodermal cells isolated from stage X embryos which are subsequently injected into the subgerminal cavity of windowed irradiated stage X white leghorn eggs. Injected eggs are sealed and incubated as described above.

### (b) Adenovirus-PEI:

Two μg of a phage phiC31 integrase expression plasmid (pCMV-int), 2 μg of a GFP expressing plasmid (pβ-actin-GFP-attB) and 2 μg of a luciferase expressing plasmid (pGLB) were incubated with 1.2 μl of JetPEI™ in 50 μl of 20 mM Hepes buffer (pH7.4). After 10 mins at 25° C., 3×10⁹ adenovirus particles (Ad5-Null, Qbiogene) were added and the incubation continued for an additional 10 mins. Embryos are transfected in ovo or ex ovo as described above.

### Example 6

### Stage I Cytoplasmic Injection

Production of transgenic chickens by cytoplasmic DNA injection using DNA injection directly into the germinal disk as described in Sang et al, Mol. Reprod. Dev., 1: 98-106 (1989); Love et al, Biotechnology, 12: 60-63 (1994) incorporated herein by reference in their entireties.

In the method of the present invention, fertilized ova, or stage I embryos, are isolated from euthanized hens 45 mins. to 4 hrs. after oviposition of the previous egg. Alternatively, eggs were isolated from hens whose oviducts have been fistulated according to the techniques of Gilbert & Wood-Gush, J. Reprod. Fertil., 5: 451-453 (1963) and Pancer et al, Br. Poult. Sci., 30: 953-7 (1989) incorporated herein in their entireties.

An isolated ovum was placed in dish with the germinal disk upwards. Ringer's buffer medium was then added to prevent drying of the ovum. Any suitable microinjection assembly and methods for microinjecting and reimplanting avian eggs are useful in the method of cytoplasmic injection of the present invention. A particularly suitable apparatus and method for use in the present invention is described in U.S. patent application Ser. No. 09/919,143, published Jul. 31, 2001, now abandoned, the disclosure of which is incorporated in its entirety herein by reference. The avian microinjection system described in the '143 application allowed the loading of a DNA solution into a micropipette, followed by prompt positioning of the germinal disk under the microscope and guided injection of the DNA solution into the germinal disk. Injected embryos could then be surgically transferred to a recipient hen as described, for example, in Olsen & Neher, J. Exp. Zool., 109: 355-66 (1948) and Tanaka et al, J. Reprod. Fertil., 100: 447-449 (1994). The embryo was allowed to proceed through the natural in vivo cycle of albumin deposition and hard-shell formation. The transgenic embryo is then laid as a hard-shell egg which was incubated until hatching of the chick. Injected embryos were surgically transferred to recipient hens via the ovum transfer method of Christmann et al in PCT/US01/26723, published Aug. 27, 2001, the disclosure of which is incorporated herein by reference in its entirety, and hard shell eggs were incubated and hatched.

Approximately 25 nl of DNA solution (about 60 ng/μl) with either integrase mRNA or protein were injected into a germinal disc of stage I White Leghorn embryos obtained 90 minutes after oviposition of the preceding egg. Typically the concentration of integrase mRNA used was 100 ng/μl, and the concentration of integrase protein was 66 ng/μl.

To synthesize the integrase mRNA, a plasmid template encoding the integrase protein was linearized at the 3′ end of the transcription unit. mRNA was synthesized, capped and a polyadenine tract added using the mMESSAGE mMACHINE T7 Ultra Kit™ (Ambion, Austin, Tex.). The mRNA was purified by extraction with phenol and chloroform and precipitated with isopropanol. The integrase protein was expressed in E. coli and purified as described by Thorpe et al, Mol. Microbiol., 38: 232-241 (2000).

A plasmid encoding for the integrase protein is transfected into the target cells. However, since the early avian embryo transcriptionally silent until it reaches about 22,000 cells, injection of the integrase mRNA or protein was expected to result in better rates of transgenesis, as shown in the Table 1 below.

The chicks produced by this procedure were screened for the presence of the injected transgene using a high throughput PCR-based screening procedure as described in Harvey et al, Nature Biotech., 20: 396-399 (2002).

TABLE 1
Summary of cytoplasmic injection results using different
integrase strategies

| Experimental | Ovum | Hard shells | Chicks | Transgenic |
|---|---|---|---|---|
| Group | transfers | produced (%) | hatched (%)* | chicks (%)^{‡} |
| No Integrase | 5164 | 3634 (70%) | 500 (14%) | 58 (11.6%) |
| Integrase | 1109 | 833 (75%) | 115 (13.8%) | 19 (16.5%) |
| mRNA | | | | |
| Integrase | 374 | 264 (70.6%) | 47 (17.8%) | 16 (34%) |
| protein | | | | |

*Percentages based on the number of hard shells
^{‡}Percentages based on the number of hatched birds

### Example 7

### Characterization of phiC31 Integrase-Mediated Integration Sites in the Chicken Genome

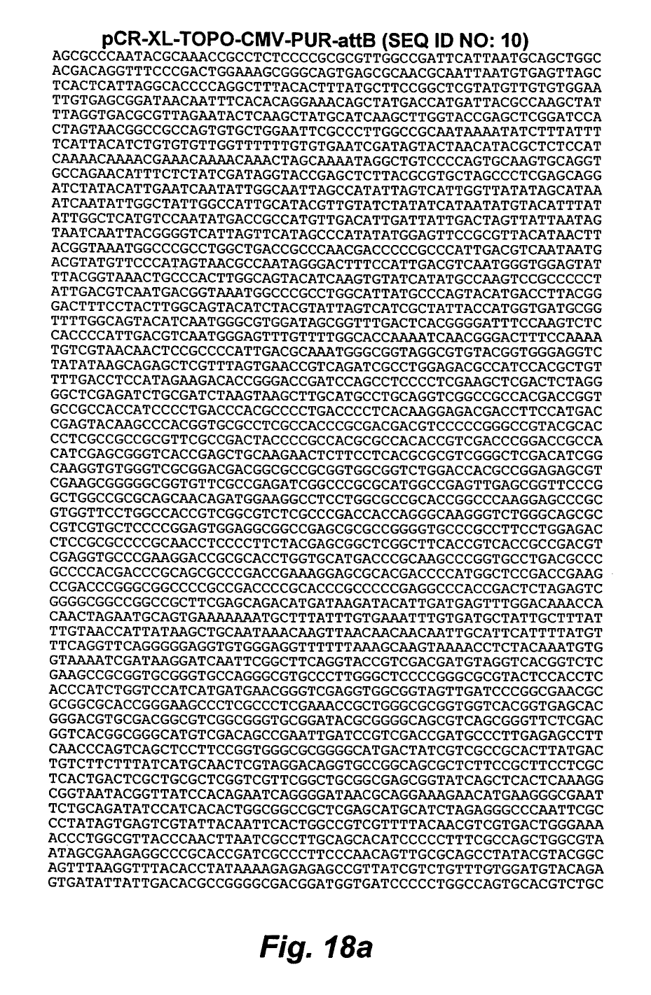

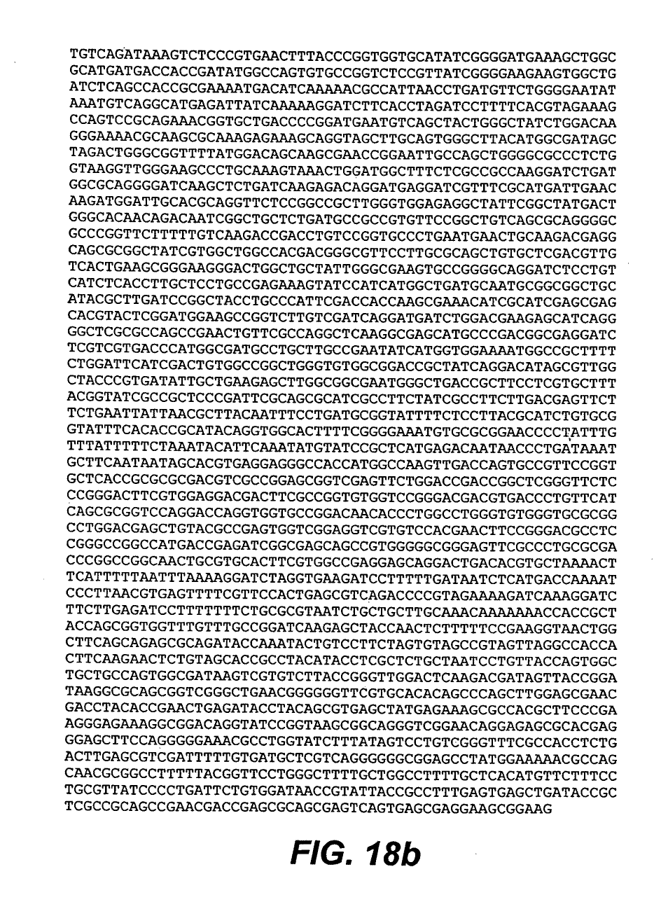

To characterize phiC31-mediated integration into the chicken genome, a plasmid rescue method was used to isolate integrated plasmids from transfected and selected chicken fibroblasts. Plasmid pCR-XL-TOPO-CMV-pur-attB (SEQ ID NO: 10, shown in FIG. 18) does not have BamH I or Bgl II restriction sites. Genomic DNA from cells transformed with pCR-XL-TOPO-CMV-pur-attB was cut with BamH I or Bgl II (either or both of which would cut in the flanking genomic regions) and religated so that the genomic DNA surrounding the integrated plasmid would be captured into the circularized plasmid. The flanking DNA of a number of plasmids were then sequenced.

DF-1 cells (chicken fibroblasts), 4×10⁵ were transfected with 50 ng of pCR-XL-TOPO-CMV-pur-attB and 1 μg of pCMV-int. The following day, the culture medium was replaced with fresh media supplemented with 1 μg/ml puromycin. After 10 days of selection, several hundred puromycin-resistant colonies were evident. These were harvested by trypsinzation, pooled, replated on 10 cm plates and grown to confluence. DNA was then extracted.

Isolated DNA was digested with BamH I and Bgl II for 2-3 hrs, extracted with phenol:chloroform:isoamyl alcohol chloroform:isoamyl alcohol and ethanol precipitated. T4 DNA ligase was added and the reaction incubated for 1 hr at room temperature, extracted with phenol:chlorofomm:isoamyl alcohol and chloroform:isoamyl alcohol, and precipitated with ethanol. 5 μl of the DNA suspended in 10 μl of water was electroporated into 25 μl of Genehogs™ (Invitrogen) in an 0.1 cm cuvette using a GenePulser II (Biorad) set at 1.6 kV, 100 ohms, 25 uF and plated on Luria Broth (LB) plates with 5 μg/ml phleomycin (or 25 μg/ml zeocin) and 20 μg/ml kanamycin. Approximately 100 individual colonies were cultured, the plasmids extracted by standard miniprep techniques and digested with Xba I to identify clones with unique restriction fragments.

Thirty two plasmids were sequenced with the primer attB—for (5′-TACCGTCGACGATGTAGGTCACGGTC-3′) (SEQ ID NO: 12) which allows sequencing across the crossover site of attB and into the flanking genomic sequence. All of plasmids sequenced had novel sequences inserted into the crossover site of attB, indicating that the clones were derived from plasmid that had integrated into the chicken genome via phiC31 integrase-mediated recombination.

The sequences were compared with sequences at GenBank using Basic Local Alignment Search Tool (BLAST). Most of the clones harbored sequences homologous to Gallus genomic sequences in the TRACE database.

### Example 8

### Insertion of a Wild-Type attP Site into the Avian Genome Augments Integrase-Mediated Integration and Transgenesis

The chicken B-cell line DT40 cells (Buerstedde et al (1990) E.M.B.O. J., 9: 921-927) are useful for studying DNA integration and recombination processes (Buerstedde & Takeda (1991) Cell, 67:179-88). DT40 cells were engineered to harbor a wild-type attP site isolated from the Streptomyces phage phiC31. Two independent cell lines were created by transfection of a linearized plasmid bearing an attP site linked to a CMV promoter driving the resistance gene to G418 (DT40-NLB-attP) or bearing an attP site linked to a CMV promoter driving the resistance gene for puromycin (DT40-pur-attP). The transfected cells were cultured in the presence of G418 or puromycin to enrich for cells bearing an attP sequence stably integrated into the genome.

A super-coiled luciferase vector bearing an attB (SEQ ID NO: 2 shown in FIG. 10) was cotransfected, together with an integrase expression vector CMV-C31int (SEQ ID NO: 1) or a control, non-integrase expressing vector (CMV-BL) into wild-type DT40 cells and the stably transformed lines DT40-NLB-attP and DT40-pur-attP.

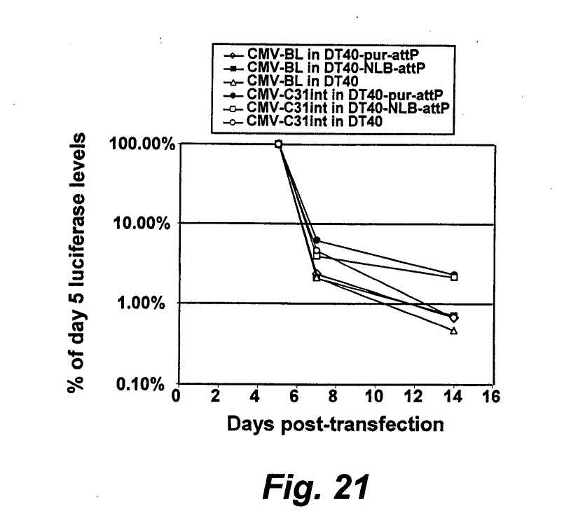

Cells were passaged at 5, 7 and 14 days post-transfection and about one third of the cells were harvested and assayed for luciferase. The expression of luciferase was plotted as a percentage of the expression measured 5 days after transfection. As can be seen in FIG. 21, in the absence of integrase, or in the presence of integrase but in the DT40 cells lacking an inserted wild-type attP site, luciferase expression from a vector bearing attB progressively decreased to very low levels. However, luciferase levels were persistent when the luciferase vector bearing attB was cotransfected with the integrase expression vector into the attP bearing cell lines DT40-NLB-attP and DT40-pur-attP. Inclusion of an attP sequence in the avian genome augments the level of integration efficiency beyond that afforded by the utilization of endogenous pseudo-attP sites.

### Example 9

### Generation of attP Transgenic Cell Line and Birds Using an NLB Vector

The NLB-attP retroviral vector is injected into stage X chicken embryos laid by pathogen-free hens. A small hole is drilled into the egg shell of a freshly laid egg, the shell membrane is cut away and the embryo visualized by eye. With a drawn needle attached to a syringe, 1 to 10 μl of concentrated retrovirus, approximately 2.5×10⁵ IU, is injected into the subgerminal cavity of the embryo. The egg shell is resealed with a hot glue gun. Suitable methods for the manipulation of avian eggs, including opening and resealing hard shell eggs are described in U.S. Pat. Nos. 5,897,998, issued May 27, 1999 and 6,397,777, issued Jun. 4, 2002, the disclosures of which are herein incorporated by reference in their entireties.

Typically, 25% of embryos hatch 21 days later. The chicks are raised to sexual maturity and semen samples are taken. Birds that have a significant level of the transgene in sperm DNA will be identified, typically by a PCR-based assay. Ten to 25% of the hatched roosters will be able to give rise to G1 transgenic offspring, 1 to 20% of which may be transgenic. DNA extracted from the blood of G1 offspring is analyzed by PCR and Southern analysis to confirm the presence of the intact transgene. Several lines of transgenic roosters, each with a unique site of attP integration, are then bred to non-transgenic hens, giving 50% of G2 transgenic offspring. Transgenic G2 hens and roosters from the same line can be bred to produce G3 offspring homozygous for the transgene. Homozygous offspring will be distinguished from hemizygous offspring by quantitative PCR. The same procedure can be used to integrate an attB or attP site into transgenic birds.

### Example 10

### Expression of Immunoglobulin Chain Polypeptides by Transgenic Chickens

Bacterial artificial chromosomes (BACs) containing a 70 kb segment of the chicken ovomucoid gene with the light and heavy chain cDNAs for a human monoclonal antibody inserted along with an internal ribosome entry site into the 3′ untranslated region of the ovomucoid gene were equipped with the attB sequence. The heavy and light chain cDNAs were inserted into separate ovomucoid BACs such that expression of an intact monoclonal antibody requires the presence of both BACs in the nucleus.

Several hens produced by coinjection of the attB-bearing ovomucoid BACs and integrase-encoding mRNA into stage I embryos produced intact monoclonal antibodies in their egg white. One hen, which had a high level of the light chain ovomucoid BAC in her blood DNA as determined by quantitative PCR particularly expressed the light chain portion of the monoclonal antibody in the egg white at a concentration of 350 nanograms per ml, or approximately 12 μg per egg.

### Example 11

### Stage I Cytoplasmic Injection with Integrase Activity and PEI

Production of transgenic chickens by cytoplasmic DNA injection directly into the germinal disk was done as described in Example 6. DNA (about 60 ng/μl) which includes a transgene was placed in approximately 25 nl of aqueous solution with integrase mRNA or integrase protein and was mixed with an equal volume of PEI that had been diluted ten fold. The mixture was injected into a germinal disc of stage I White Leghorn embryos obtained about 90 minutes after oviposition of the preceding egg. Typically the concentration of integrase mRNA used was about 100 ng/μl, and the concentration of integrase protein was about 66 ng/μl. The integrase mRNA was synthesized according to Example 6.

Transgenic chicks produced by this procedure using: integrase mRNA/PEI and integrase protein/PEI showed positive results for the presence of heterologously expressed protein in the blood, semen and egg white.

### Example 12

### Stage I Cytoplasmic Injection with Integrase Activity and NLS

Production of transgenic chickens by cytoplasmic DNA injection directly into the germinal disk was done as described in Example 6.

DNA which includes a transgene was suspended in 0.25 M KCl and SV40 T antigen nuclear localization signal peptide (NLS peptide, amino acid sequence CGGPKKKRKVG (SEQ ID NO: 13)) was added to achieve a peptide DNA molar ratio of 100:1. The DNA (about 60 ng/μl) was allowed to associate with the SV40 T antigen NLS peptide by incubating at 25 degrees C. for about 15 minutes.

Integrase mRNA or integrase protein was added to approximately 25 nl of an aqueous DNA/NLS solution, typically, to produce a final concentration of integrase mRNA of about 50 ng/μl, or an integrase protein concentration of about 33 ng/μl. The mixture was injected into a germinal disc of stage I White Leghorn embryos obtained about 90 minutes after oviposition of the preceding egg. The integrase mRNA was synthesized as according to Example 6.

Transgenic chicks produced by this procedure using: integrase mRNA/NLS and integrase protein/NLS showed positive results for the presence of heterologously expressed protein in blood, semen and egg white.

### Example 13

### Dispersing of Plasmid DNA in Avian Stage I Embryos

DNA samples are Cy3 labeled with a Cy3 ULS labeling kit (Amersham Pharmacia Biotech). Briefly, plasmid DNA (1 μg) was sheared to approximately 100 to 500 bp fragments by sonication. Resulting DNA was incubated at 65° C. for 15 min in Cy3 ULS labeling solution and unincorporated Cy3 dye was removed by spin column chromatography (CentriSep, Princeton Separations). The distribution of the DNA in stage I avian embryos was visualized after introduction into the stage I avian embryo. Enough high molecular weight or low molecular weight PEI was added to the DNA to coat the DNA. Typically, PEI was added to the DNA to a concentration of about 5%. Any useful volume of DNA/PEI can be used, for example about 25 nl.

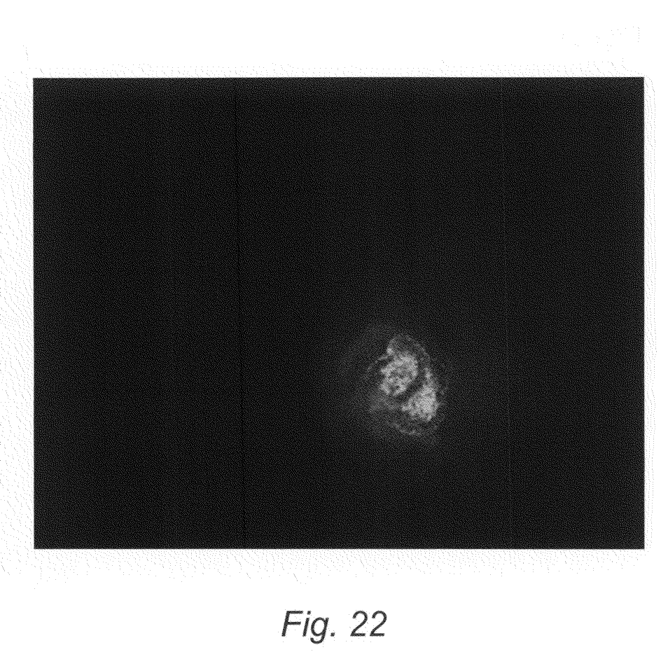

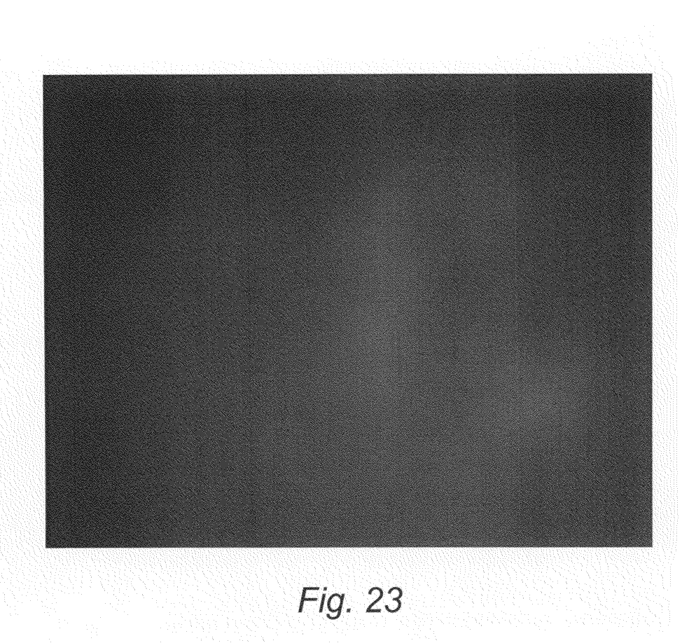

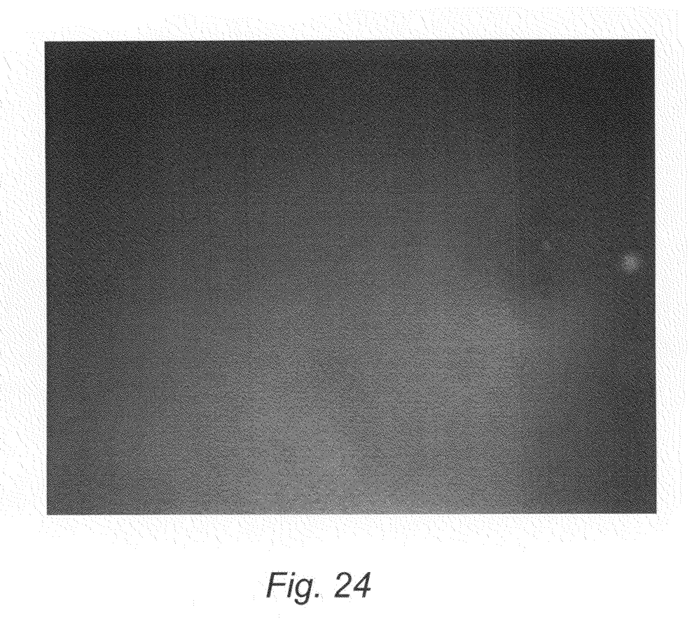

FIG. 22 shows an avian stage one embryo containing Cy3 labeled naked DNA. In FIG. 22 it can be seen that the DNA is localized to certain areas of the embryo. FIG. 23 and FIG. 24 show an avian stage one embryo containing Cy3 labeled DNA coated with low molecular (22 kD) weight PEI (FIG. 23) and high molecular weight (25 kD) PEI (FIG. 24). In FIGS. 23 and 24, it can be seen that the DNA is dispersed throughout the embryos.

These experiments show that DNA/PEI conjugates are distributed more uniformly in the cytoplasm of injected embryos when compared with naked DNA.

### Example 14

### Production of an attP Transgenic Chicken

G0 transgenic chickens have been produced as described in Example 9. Several hundred stage X White Leghorn eggs were injected with the NLB-attP vector and about 50 chicks hatched. Sperm from approximately 30% of the hatched roosters has been shown to be positive for the attP site. These hemizygotic chickens are used to generate transgenic G2 chickens homozygotic for the attP site.

### Example 15

### Cytoplasmic Injection of attP Stage I Embryos with OMC24-attB-IRES-CTLA4

Transgenic chickens are produced by cytoplasmic DNA injection directly into the germinal disk of eggs laid by transgenic homozygous attP chickens and fertilized with sperm from the same line of homozygous attP roosters, the line produced as described in Example 14. The cytoplasmic injections are carried out as described in U.S. patent application Ser. No. 09/919,143, filed Jul. 31, 2001, ('143 application), now abandoned and U.S. patent application Ser. No. 10/251,364, filed Sep. 18, 2002, now issued U.S. Pat. No. 7,312,374. The disclosures of each of these two patent applications are incorporated herein by reference in their entirety.

Stage I embryos are isolated 45 mins. to 4 hrs. after oviposition of the previous egg. An isolated embryo is placed in a dish with the germinal disk upwards. Ringer's buffer medium is added to prevent drying of the ovum. The avian microinjection system described in the '143 application allows for the loading of DNA solution into a micropipette, followed by prompt positioning of the germinal disk under the microscope and guided injection of the DNA solution into the germinal disk.

Approximately 25 nl of a DNA solution (about 60 ng/μl) of the 77 kb OMC24-attB-IRES-CTLA4, disclosed in U.S. patent application Ser. No. 10/856,218, filed May 28, 2004, now issued U.S. Pat. No. 7,294,507, the disclosure of which is incorporated in its entirety herein by reference, with either integrase mRNA or protein are injected into a germinal disc of the isolated stage I embryos. Typically, the concentration of integrase mRNA used is 100 ng/μl or the concentration of integrase protein is 66 ng/μl.

To synthesize the integrase mRNA, a plasmid template encoding the integrase protein is linearized at the 3′ end of the transcription unit. mRNA is synthesized, capped and a polyadenine tract added using the mMESSAGE mMACHINE T7 Ultra Kit™ (Ambion, Austin, Tex.). The mRNA is purified by extraction with phenol and chloroform and precipitated with isopropanol. The integrase protein is expressed in E. coli and purified as described by Thorpe et al, Mol. Microbiol., 38: 232-241 (2000).

Injected embryos are surgically transferred to a recipient hen as described in Olsen & Neher, J. Exp. Zool., 109: 355-66 (1948) and Tanaka et al, J. Reprod. Fertil., 100: 447-449 (1994). The embryo is allowed to proceed through the natural in vivo cycle of albumin deposition and hard-shell formation. The transgenic embryo is then laid as a hard-shell egg which is incubated until hatching of the chick. Injected embryos are surgically transferred to recipient hens via the ovum transfer method of Christmann et al in PCT/US01/26723, published Aug. 27, 2001, the disclosure of which is incorporated by reference in its entirety, and hard shell eggs are incubated and hatched.

The chicks produced by this procedure are screened for the presence of the injected transgene using a high throughput PCR-based screening procedure as described in Harvey et al, Nature Biotech., 20: 396-399 (2002). Approximately 20% of the chicks are positive for the transgene. Eggs from each of the mature hens carrying the transgene are positive for CTLA4.

### Example 16

### Cytoplasmic Injection of attP Stage I Chicken Embryos with OM10-attB-CTLA4

Transgenic chickens are produced by cytoplasmic DNA injection directly into the germinal disk of eggs laid by transgenic homozygous attP chickens and fertilized with sperm from the same line of homozygous attP roosters essentially as described in Example 15.

Approximately 25 nl of a 60 ng/μl DNA solution of the OMC24-attB-IRES-CTLA4 construct of Example 15 with the OMC24 70 kb ovomucoid gene expression controlling region and IRES of the construct replaced with the 10 kb ovomucoid gene expression controlling region of pBS-OVMUP-10, also disclosed in U.S. patent application Ser. No. 10/856,218, filed May 28, 2004, now issued U.S. Pat. No. 7,294,507 is injected into a fertilized germinal disc of stage I embryos along with and integrase protein. The concentration of integrase protein used is 66 ng/μl.

Injected embryos are then surgically transferred to a recipient hen, hard shell eggs are produced, incubated and hatched. Approximately 30% of the chicks are positive for the transgene. Eggs from each of the mature hens carrying the transgene are positive for CTLA4.

### Example 17

### Production of attP Transgenic Quail Using an NLB Vector

The NLB-attP retroviral vector is injected into stage X quail embryos laid by pathogen-free quail. A small hole is drilled into the egg shell of a freshly laid egg, the shell membrane cut away and the embryo visualized by eye. With a drawn needle attached to a syringe, 1 to 10 μl of concentrated retrovirus, approximately 1.0×10⁵ IU, is injected into the subgerminal cavity of the embryo. The egg shell is resealed with a hot glue gun.

Typically, 25% of embryos hatch. The chicks are raised to sexual maturity and semen samples are taken. Birds that have a significant level of the transgene in their sperm DNA will be identified, typically by a PCR-based assay. Of the hatched G0 male quail, about 1% to about 20% are transgenic. The transgenic G0 quail are bred to nontransgenic quail to produce hemizygotic G1 offspring. DNA extracted from the blood of G1 offspring is analyzed by PCR and Southern analysis to confirm the presence of the intact transgene. Several lines of hemizygotic transgenic male quail, each with a unique site of attP integration, are then bred to non-transgenic quail giving G2 offspring, 50% of which are transgenic. Transgenic G2 male and female from the same line are then bred to produce G3 offspring homozygous for the transgene. Homozygous offspring are distinguished from hemizygous offspring by quantitative PCR.

### Example 18

### Cytoplasmic Injection of attP Stage I Quail Embryos with OMC24-attB-IRES-G-CSF

Transgenic quail are produced by cytoplasmic DNA injection directly into the germinal disk of eggs laid by fully transgenic homozygous attP quail produced as described in Example 17. The cytoplasmic injections are carried out essentially as described in the '143 application and U.S. patent application Ser. No. 10/251,364, filed Sep. 18, 2002, now issued U.S. Pat. No. 7,312,374.

Stage I embryos from homozygous attP quail fertilized with sperm from a homozygous attP quail are isolated approximately 90 minutes after oviposition of the previous egg. An isolated embryo is placed in a dish with the germinal disk upwards. Ringer's buffer medium is added to prevent drying of the ovum. The avian microinjection system described in the '143 application is used to inject approximately 25 nl of a DNA solution (about 60 ng/μl) of OMC24-attB-IRES-CTLA4, with the CTLA coding sequence replaced with the coding sequence for a human-granulocyte colony stimulating factor, and integrase protein into the germinal disc of the stage I quail embryos. The concentration of integrase protein used is 66 ng/μl.

Injected embryos are surgically transferred to a recipient quail essentially as described in Olsen & Neher, J. Exp. Zool., 109: 355-66 (1948) and Tanaka et al, J. Reprod. Fertil., 100: 447-449 (1994). The embryo is allowed to proceed through the natural in vivo cycle of albumin deposition and hard-shell formation. The transgenic embryo is then laid as a hard-shell egg which is incubated until hatching of the chick.

The chicks produced by this procedure are screened for the presence of the injected transgene using a high throughput PCR-based screening procedure as described in Harvey et al, Nature Biotech., 20: 396-399 (2002). Approximately 20% of the chicks are positive for the transgene. Eggs from each of the mature female quail carrying the transgene are positive for G-CSF.

### Example 19

### Generation of attP Transgenic Duck Using an NLB Vector

The NLB-attP retroviral vector is injected into stage X Duck embryos laid by pathogen-free Ducks. A small hole is drilled into the egg shell of a freshly laid egg, the shell membrane cut away and the embryo visualized by eye. About 1 to 10 μl of concentrated retrovirus, approximately 2.5×10⁵ IU, is injected into the subgerminal cavity of the embryo. The egg shell is resealed with a hot glue gun.

Homozygous G3 offspring are obtained essentially as described in Example 17 for quail.

### Example 20

### Stage I Cytoplasmic Injection of attP Stage I Duck Embryos with OM24-attB-IRES-CTLA4

Transgenic ducks are produced by cytoplasmic DNA injection directly into the germinal disk of eggs laid by homozygous attP ducks fertilized with sperm from homozygous attP ducks. The injection of the stage I embryos is carried out essentially as described in the '143 application and U.S. patent application Ser. No. 10/251,364, filed Sep. 18, 2002, now issued U.S. Pat. No. 7,312,374. Approximately 25 nl of a DNA solution (about 60 ng/μl) of OMC24-attB-IRES-CTLA4, with the CTLA4 coding region replaced with a coding sequence for human erythropoietin, and integrase encoding mRNA and protein is injected into the germinal disc of the stage I embryos. The concentration of integrase mRNA used is 100 ng/μl. The injected embryos are surgically transferred to a recipient duck and the embryo is allowed to proceed through the natural in vivo cycle of albumin deposition and hard-shell formation. The transgenic embryo is laid as a hard-shell egg which is incubated until hatching and the chicks are screened for the presence of the injected transgene. Approximately 20% of the chicks are positive for the transgene. Eggs from each of the mature female ducks carrying the transgene are positive for erythropoietin.

### Example 21

### Production of Transchromosomic Chickens Using Satellite DNA-Based Artificial Chromosomes

Satellite DNA-based artificial chromosomes (ACEs, as described in Lindenbaum et al Nucleic Acids Res (2004) vol 32 no. 21 e172) were isolated by a dual laser high-speed flow cytometer as described previously (de Jong, G, et al. Cytometry 35: 129-133, 1999).

The flow-sorted chromosomes were pelleted by centrifugation of a 750 μl sample containing approximately 10⁶ chromosomes at 2500×g for 30 min at 4° C. The supernatant, except the bottom 30 microliters (μl) containing the chromosomes, was removed resulting in a concentration of about 7000 to 11,500 chromosomes per μL of injection buffer (Monteith, et al. Methods Mol Biol 240: 227-242, 2004). Depending on the number of chromosomes to be injected, 25-100 nanoliters (nl) of injection buffer was injected per embryo.

Embryos for this study were collected from 24-36 week-old hens from commercial White Leghorn variety of G. gallus. Embryo donor hens were inseminated weekly using pooled semen from roosters of the same breed to produce eggs for injection.

On the day of egg collection, fertile hens were euthanized 2 h post oviposition by cervical dislocation. Typically, oviposition is followed by ovulation of the next egg after about 24 minutes (Morris, Poultry Science 52: 423-445, 1973). The recently ovulated and fertilized eggs were collected from the upper magnum region of the oviduct under sterile conditions and placed in a glass well and covered with Ringers' Medium (Tanaka, et al. J Reprod Fertil 100: 447-449, 1994) and maintained at 41° C. until microinjection.

Cytoplasmic injection of artificial chromosomes was achieved using the microinjection apparatus disclosed in U.S. patent application Ser. No. 09/919,143, filed Jul. 31, 2001, now abandoned. Chromosomes were injected into the Stage I embryos at a single site. Each embryo was cytoplasmically injected with approximately: 175, 250, 350, 450, 550, 800 or >1000 chromosomes. The chromosomes were injected in a suspension of 25-100 nanoliters (nl) of injection buffer.

Following microinjection, the embryos were transferred to the oviduct of recipient hens using an optimized ovum transfer (OT) procedure (Olsen, M and Neher, B. J Exp Zool 109: 355-66, 1948), with the exception that the hens were anesthetized by Isofluorane gas. Typically, about 26 h after OT, the recipient hens lay a hard shell egg containing the manipulated ovum. Eggs were incubated for 21 days in a regular incubator until hatching of the birds.

The chromosomes were injected into the embryos over a 9 day period. The chromosomes were divided into three batches for delivery to the embryos each batch being injected over a three day period. Chromosomes were introduced into the embryos by a single injection using the microinjection assembly disclosed in the '143 patent application. Following injection, each egg was transferred to a recipient hen. A total of 301 transfers were performed, resulting in 226 (75%) hard shells and 87 hatched chicks (38%, see Table 2).

TABLE 2
Hatching of embryos microinjected with satellite
DNA-based artificial chromosomes.

| | Ovum | Hard shells | | | |
|---|---|---|---|---|---|
| | transfers | produced | | hatched birds | |
| 1^{st} batch | 71 | 53 | | 15 | |
| 2^{nd} batch | 113 | 80 | | 33 | |
| 3^{rd} batch | 117 | 93 | | 39 | |
| Totals | 301 | 226 | (75%) | 87 | (38%) |

Previous experiments have determined that hatching is not significantly affected when embryos were injected with up to 100 nl of injection buffer. Satellite DNA-based artificial chromosomes were injected in suspensions of between 25-100 nl of injection buffer.

As discussed, the embryos were injected with one of seven different numbers of artificial chromosomes. There was shown to be a correlation between the number of chromosomes injected per egg and the hatch rate. All transchromosomic birds in the present study were obtained from embryos injected with 550 chromosomes or less (see Table 3). There was no significant difference in the hatching rates observed between the experimental groups (batches 1, 2 and 3).

Six transchromosomic founders were produced based on two separate PCR analysis (6.8%, see Table 3) using primers which anneal to the puromycin resistance gene (about 75 copies of the purR gene are present on the chromosome. All positive birds appear normal.

TABLE 3
Effect of the number of Chromosomes injected per embryo on hatching
and number of transchromosomic birds produced.

| # chromosomes | | | |
|---|---|---|---|
| injected | # of hard | # chicks | # of positive |
| per embryo | shells | hatched | birds (bird tag #) |
| 175 | 31 | 11 (35%) | 3 (BB7478, BB7483, BB7515) |
| 250 | 51 | 25 (49%) | 1 (BB 7499) |
| 350 | 15 | 6 (40%) | 0 |
| 450 | 31 | 11 (35%) | 0 |
| 550 | 39 | 17 (43%) | 2 (BB7477, BB7523) |
| 800 | 26 | 5 (19%)* | 0 |
| 1000 | 33 | 10 (30%)* | 0 |
| Totals | 226 | 87 (38%) | 6 (6.8%) |

*hatching rates of embryos injected with >550 chromosomes was significantly lower (p < 0.025)

To confirm the PCR results, erythrocytes from all PCR-positive birds as well as fibroblast cells derived from skin biopsies of 5 PCR-positive birds were analyzed by interphase and metaphase FISH using a mouse-specific major satellite DNA probe (Co, et al. Chromosome Res 8: 183-191, 2000). Five of the six chicks (5.3% out of total number of chicks analyzed) tested by FISH were positive in at least one cell type (see Table 4) at 3 weeks of age. FISH analysis of erythrocytes was repeated when the birds reached 8 weeks of age and had tripled their body weight. Similar numbers of artificial chromosome-positive cells found in each bird were observed in this second FISH analysis.

TABLE 4
Summary of FISH analysis of Red Blood Cells (RBCs) and fibroblast
cells derived from transchromosomic birds. Fibroblast cells from
hen # 7515 were not available for analysis.

| | | % of artificial | % of artificial |
|---|---|---|---|
| | | chromosome | chromosome |
| | Sex of | positive | positive fibroblasts |
| Bird # | Bird | RBCs by FISH | by FISH |
| BB7499 | Female | 77% | 87% |
| BB7483 | Female | 0.8% | 0% |
| BB7477 | Male | 3% | 2.8% |
| BB7478 | Male | 15% | 3% |
| BB7515 | Female | 1.3% | NA |
| BB7523 | Male | 0% | 0% |
| Neg. control | — | 0% | 0% |

To verify the chromosomes were intact, metaphase spreads from fibroblast cells derived from founders were made as described previously (Garside and Hillman (1985) Experientia 41: 1183-1184). FISH analysis of metaphase spreads using the major satellite DNA probe showed the artificial chromosomes appear intact, with no apparent fragmentation or translocation onto the chicken's chromosomes. FISH analysis using a mouse minor satellite probe, which detects the centromeric region of the introduced chromosomes (Wong and Rattner (1988) J. Nucleic Acids Res 16: 11645-11661), demonstrated the centromere of the chromosomes was intact. Furthermore, the percentage of satellite DNA-based artificial chromosomes-positive cells from metaphase spreads agreed closely to those observed in interphase FISH.

Analysis of G1 embryos from test bird BB7499 has shown the artificial chromosome to be transmitted through the germline. In addition, sperm from BB7499 was shown to test positive for the artificial chromosome which will also provide for germline transmission of the artificial chromosome.

### Example 22

### Production of EPO and G-CSF Vectors for the Production of Transchromosomic Chickens

Two vectors were constructed for introduction into Satellite DNA-based artificial chromosomes. 10MC24-IRES1-EPO—ChromattB was constructed by inserting an EPO coding sequence into an OMC24-IRES BAC clone disclosed in U.S. patent application Ser. No. 10/856,218, filed May 28, 2004, now issued U.S. Pat. No. 7,294,507, the disclosure of which is incorporated in its entirety herein by reference. The EPO coding sequence was inserted in the clone so as to be under the control of the ovomucoid promoter. That is, the EPO coding sequence was inserted in place of the LC portion of OMC-IRES-LC. An attB site and a hygromycin^{R} coding sequence were also inserted into the vector in such a manner as to facilitate recombination into an attP site in a SATAC artificial chromosome (i.e., ACE), as see in FIG. 25. The attP site in the SATAC is located adjacent to an SV40 promoter which provides for expression of the hygromycin^{R} coding sequence upon integration of the vector into the attP site allowing for selection of cells containing a recombinant artificial chromosome (see, for example, U.S. Pat. No. 6,743,967, issued Jun. 1, 2004; U.S. Pat. No. 6,025,155, issued Feb. 15, 2000 and Lindenbaum et al Nucleic Acids Res (2004) vol 32 no. 21 e172 (see FIG. 25), the disclosure of each of these two patents and the publication are incorporated in their entirety herein by reference).

A coding sequence for G-CSF, which was codon optimized for expression in chicken tubular gland cells, was inserted in the 10MC24-IRES1-EPO—ChromattB construct in place of the EPO coding sequence to produce 10MC24-IRES-GCSF—ChrommattB.

### Example 23

### Production of Erythropoietin and G-CSF Using Artificial Chromosomes in Chickens

Cells containing the recombinant artificial chromosome are produced and identified as described in Lindenbaum et al Nucleic Acids Res (2004) Vol 32 no. 21 e172. Briefly, 2.5 μg of 10MC24-IRES1-EPO ChromattB and 2.5 μg of an expression vector which contains a lambda integrase gene (int) having a codon mutation at position 174 to substitute a lysine for a glutamine (pCXLamROK, see Lindenbaum et al Nucleic Acids Res (2004) vol 32 no. 21 e172) are transfected by standard lipofection methodologies into LMTK-cells which contain the platform SATAC (ACE) (A of FIG. 25). DNA comprising the nucleotide sequence of interest, in this case 10MC24-IRES1-EPO ChromattB, that has been highly purified, for example, utilizing a CsCl gradient centrifugation as is well known in the art, is particularly useful, though not required. Hygromycin resistant cells clones are identified by standard antibiotic selection methodologies.

Recombinant chromosomes are prepared from the cells and isolated by flow cytometry. The substantially purified artificial chromosomes are introduced into chickens by microinjection into stage one embryos as disclosed in U.S. patent application Ser. Nos. 10/679,034, filed Oct. 2, 2003 and 09/919,143, filed Jul. 31, 2001, now abandoned. Resulting chimeric germline transchromosomal avians can be identified by any useful method such as Southern blot analysis.

### Example 24

### Production of a Monoclonal Antibody Using Drosophila Artificial Chromosomes in Turkey

Artificial chromosomes comprising a Drosophila chromosome centromere (DAC) are prepared essentially using methods described in U.S. Pat. No. 6,025,155, issued Feb. 15, 2000, the disclosure of which is incorporated in its entirety herein by reference.

An attB site and a hyrgromycin^{R} coding sequence are inserted into the OMC24-IRES-LC and OMC24-IRES-HC vectors disclosed in U.S. patent application Ser. No. 10/856,218, filed Jul. 31, 2001, now issued U.S. Pat. No. 7,294,507, the disclosure of which is incorporated in its entirety herein by reference, which are then each cloned into a DAC essentially as described in Examples 22 and 23. The recombinant DACs are prepared and then isolated by a dual laser high-speed flow cytometer.

The flow-sorted chromosomes are pelleted by centrifugation and are diluted to a concentration of about 7000-12,000 chromosomes per μl of injection buffer. Approximately 50 nanoliters (nl) of injection buffer is injected per turkey embryo.

Embryos for this study are collected from actively laying commercial turkeys. Embryo donor turkeys are inseminated weekly using pooled semen from male turkeys of the same breed to produce eggs for injection.

On the day of egg collection, fertile hens are euthanized 2 h post oviposition by cervical dislocation. The recently ovulated and fertilized eggs are collected from the upper magnum region of the oviduct under sterile conditions and placed in a glass well and covered with Ringers' Medium and maintained at about 40° C. until microinjection.

Cytoplasmic injection of artificial chromosomes containing the OMC24-IRES-LC is achieved using the microinjection apparatus disclosed in U.S. patent application Ser. No. 09/919,143, now abandoned. Approximately 500 chromosomes are injected into the Stage I embryos at a single site.

Following microinjection, the embryos are transferred to the oviduct of recipient turkeys essentially as described in Olsen et al, B. J Exp Zool 109: 355-66, 1948. Typically, about one day after OT, the recipient turkeys lay a hard shell egg containing the manipulated ovum. Eggs are incubated in an incubator until hatching of the birds.

G2 transchromosomal turkeys are obtained which contain the artificial chromosome in their genome. The artificial chromosome containing the OMC24-IRES-HC is introduced into embryos obtained from the G2 turkeys in essentially the same manner as described for the OMC24-IRES-LC.

Eggs from G1 transchromosomal turkeys which contain both the OMC-IRES-LC and OMC24-IRES-HC containing chromosomes in their genome are tested for the presence of intact functional monoclonal antibody. A Costar flat 96-well plate is coated with 100 μl of C Goat-anti-Human kappa at a concentration of 5 μg/ml in PBS. The plate is incubated at 37° C. for two hours. 200 μl of 5% PBA is added to the wells followed by an incubation at 37° C. for about 60-90 minutes followed by a wash. 100 μl of egg white samples (diluted in 1% PBA:LBP) is added to each well and the plate is incubated at 37° C. for about 60-90 min followed by a wash. 100 μl of a 1:2000 dilution of F′2 Goat anti-Human IgG Fc-AP in 1% PBA is added to the wells and the plate is incubated at 37° C. for 60-90 min followed by a wash. The antibody is detected by placing 75 μl of 1 mg/ml PNPP (p-nitrophenyl phosphate) in 5× developing buffer in each well and incubating for about 10-30 mins at room temperature. The detection reaction is stopped using 75 μl of 1N NaOH. The egg white tests positive for significant levels of the antibody.

### Example 25

### Production of Interferon Using Avian Artificial Chromosomes in Quail

Artificial chromosomes comprising a chicken (Barred-Rock) chromosome centromere (CAC) are prepared essentially using methods described in U.S. Pat. No. 6,743,967, issued Jun. 1, 2004, the disclosure of which is incorporated in its entirety herein by reference.

A coding sequence for interferon alpha 2b disclosed in U.S. patent application Ser. No. 10/463,980, filed Jun. 17, 2003, the disclosure of which is incorporated in its entirety herein by reference, is inserted in the 10MC24-IRES1-Epo—ChromattB construct disclosed herein in Example 22 in place of the EPO coding sequence to produce 10MC24-IRES-INF—ChrommattB. The 10MC24-IRES-INF—ChrommattB is cloned into the CACs essentially as described in Example 23. The recombinant CACs are prepared then isolated by a dual laser high-speed flow cytometer.

The flow-sorted chromosomes are pelleted by centrifugation and are diluted to a concentration of about 10,000 chromosomes per μl of injection buffer. Approximately 50 nanoliters (nl) of injection buffer is injected per quail embryo.

Embryos for this study are collected from actively laying quail. Embryo donor quail are inseminated weekly using pooled semen from male quail of the same breed to produce eggs for injection.

On the day of egg collection, fertile quail are euthanized 2 h post oviposition by cervical dislocation. The recently ovulated and fertilized eggs are collected from the upper magnum region of the oviduct under sterile conditions and placed in a glass well and covered with Ringers' Medium and maintained at about 40° C. until microinjection.

Cytoplasmic injection of artificial chromosomes is achieved using the microinjection apparatus disclosed in U.S. patent application Ser. No. 09/919,143, filed Jul. 31, 2001, now abandoned. Chromosomes are injected into the Stage I embryos at a single site in each embryo.

Following microinjection, the embryos are transferred to the oviduct of recipient quail essentially as described in Olsen et al, B. J Exp Zool 109: 355-66, 1948. Typically, about one day after OT, the recipient quail lay a hard shell egg containing the manipulated ovum. Eggs are incubated in an incubator until hatching of the birds.

Eggs from G2 transchromosomal quail test positive for the presence of intact functional interferon alpha 2b.

### Example 26

### Production of Monoclonal Antibody Using Avian Artificial Chromosomes in Chicken

An attB site and a hygromycin^{R} coding sequence are inserted into the OMC24-IRES-LC and OMC24-IRES-HC vectors disclosed in U.S. patent application Ser. No. 10/856,218, filed Jul. 31, 2001, now issued U.S. Pat. No. 7,294,507 which are then each cloned into CACs of Example 25 essentially as described in Examples 22 and 23. The CACs are isolated by a dual laser high-speed flow cytometer.

The flow-sorted chromosomes are pelleted by centrifugation and are diluted to a concentration of 7000-12,000 chromosomes per μl of injection buffer. Approximately 50 nanoliters (nl) of injection buffer is injected per chicken embryo.

Embryos for this study are collected from actively laying G. gallus. Embryo donor chickens are inseminated weekly using pooled semen from male chickens of the same breed to produce eggs for injection.

On the day of egg collection, fertile hens are euthanized 2 h post oviposition by cervical dislocation. The recently ovulated and fertilized eggs are collected from the upper magnum region of the oviduct under sterile conditions and placed in a glass well and covered with Ringers' Medium and maintained at about 41° C. until microinjection.

Cytoplasmic injection of artificial chromosomes containing the OMC24-IRES-LC is achieved using the microinjection apparatus disclosed U.S. patent application Ser. No. 09/919,143, now abandoned. Approximately 500 chromosomes are injected into the Stage I embryos at a single site.

Following microinjection, the embryos are transferred to the oviduct of recipient chickens essentially as described in Olsen et al, B. J Exp Zool 109: 355-66, 1948. Typically, about one day after OT, the recipient chickens lay a hard shell egg containing the manipulated ovum. Eggs are incubated in an incubator until hatching of the G0 birds.

G2 transchromosomal chickens are obtained which contain the artificial chromosome in their genome. The artificial chromosome containing the OMC24-IRES-HC is introduced into embryos obtained from the G2 chickens in essentially the same manner as described for the OMC24-IRES-LC.

Eggs from G1 transchromosomal chickens which contain both the OMC-IRES-LC and OMC24-IRES-HC in their genome are tested for the presence of intact functional monoclonal antibody. A Costar flat 96-well plate is coated with 100 ul of C Goat-anti-Human kappa at a concentration of 5 μg/ml in PBS. The plate is incubated at 37° C. for two hours. 200 μl of 5% PBA is added to the wells followed by an incubation at 37° C. for about 60-90 minutes followed by a wash. 100 ul of egg white samples (diluted in 1% PBA:LBP) is added to each well and the plate is incubated at 37° C. for about 60-90 min followed by a wash. 100 ul of a 1:2000 dilution of F′2 Goat anti-Human IgG Fc-AP in 1% PBA is added to the wells and the plate is incubated at 37° C. for 60-90 min followed by a wash. The antibody is detected by placing 75 ul of 1 mg/ml PNPP (p-nitrophenyl phosphate) in 5× developing buffer in each well and incubating for about 10-30 mins at room temperature. The detection reaction is stopped using 75 ul of 1N NaOH. The egg white tests positive for significant levels of the antibody.

### Example 27

### Cell Culture and Transfection for the Production of an Insert Containing Artificial Chromosome and Screening for Positive Clones

pK161 is a cosmid containing a 8.2 kb mouse rDNA insert. The plasmid is produced as disclosed in Csonka et al 2000, Journal of Cell Science 113, 3207-3216. 100 μg of cosmid pK161 is digested with Cla I, purified by phenol/chloroform extraction and ethanol precipitation then resuspended at approximately 1 μg/μl in TE, pH 8.0. YAC DNA containing the human light-chain and heavy-chain immunoglobulin loci shown in FIGS. 27A and 27B are prepared as disclosed in Example 30.

LMTK-cells (obtained from ATCC) are cultured at 37° C. in 5% CO2 in a humidified incubator in DMEM (Invitrogen), 10% FBS (Hyclon) (LMTK-media). Prior to the day of transfection, ten 10 cm plates are seeded with approximately 2×10⁶ cells per dish. Transfection with ExGen 500 (i.e., sterile 0.01 mM 22 kDa polyethylenimine (PEI), Fermentas Life Sciences) can be performed according to the manufacturers instructions or as follows.

On the day of the transfection, LMTK-cells are washed once with 3 ml of Optimem and the media is replaced with 6 ml of Optimem. In an eppendorf tube, 250 μl of HBS (150 mM NaCl, 20 mM HEPES, pH 7.4) is mixed with 3.6 μl of ExGen 500. In a second tube, 250 μl of HBS is mixed with 6 μg of linearized pFK161, 3.0 μg of gel-purified kappa light chain YAC and 3.0 μg of gel-purified heavy chain YAC.

The PEI (ExGen) mixture is added dropwise to the DNA mixture, without mixing of the two solutions.

After incubation at RT for 10 min, the solution is gently mixed by pipeting up and down with a wide-bore pipet 3 times. 50 μl of the transfection mix is added to each 10 cm dish of LMTK-cells and the plates are swirled to distribute the DNA/PEI complexes. 4-6 hours post-transfection, the media is replaced with 10 ml of LMTK-media. 48 hours post-transfection, the media is replaced with LMTK-media plus 200 μg/ml G418 (Geneticin, Invitrogen). The selective media is replaced every 2-3 days until colonies are apparent.

Fifty G418-resistant colonies are isolated with cloning cylinders and are transferred to single wells in 24-well tissue culture plates. When the clones are at or near confluency, they trypsinized and split into three 24-well plates.

To determine which clones carry a desired artificial chromosome, metaphase or interphase FISH is performed. Purified light chain YAC DNA is labeled with biotin-14dCTP by random priming (Bioprime DNA labeling system, Invitrogen). The heavy chain YAC DNA is labeled with digoxigenin-11 dUTP by random priming (Dig High Prime, Roche Diagnostics). The heavy and light chain YAC probes are mixed and hybridized to metaphase chromosomes or interphase nuclei. The hybridized biotin signals are made visible with fluorescein labeled avidin, and the digoxigenin signals are visualized with rhodamine labeled anti-digoxigenin antibody following standard protocols. The nuclei or chromosomes are counterstained with DAPI and visualized on an Olympus IX70 microscope configured with DAPI, FITC and rhodamine fluorescent excitation filters.

Two clones are found to have an episomal element indicative of an artificial chromosome. Both clones are positive for the heavy and light chain YACs, indicating that both YACs are incorporated into the artificial chromosomes. The artificial chromosomes are believed to be satellite artificial chromosomes.

### Example 28

### Copy Number Determination of Ig Loci Inserts and Determination of Structural Integrity of the Loci in the Artificial Chromosomes

In order to simplify the interpretation of the analysis of structural integrity of the Ig containing YACs, it is desirable to obtain artificial chromosomes which carry one copy of each YAC. Real time PCR using Taqman® chemistry is utilized to identify clones containing a single copy of the YACs. Several primer/probe sets are designed to detect each YAC. The amplicon detection probes are labeled using FAM as the dye and TAMRA as the quencher. 10 ng of genomic DNA purified from the positive clones that are identified in Example 30 are assayed in a 30 μl reaction using the TaqMan® Fast Universal PCR Master Mix, No AmpErase® UNG and 7900HT (Applied Biosystems). Amplification curves are compared to standards that are composed of differing amounts of purified YACs in the presence of 10 ng of LMTK-DNA. Both positive clones of Example 27 appear to have a single copy of the light chain YAC as is indicated by overlap of the amplification curves and the Ct value relative to the standard curve. One clone appears to have two or more copies of the heavy chain YAC as the amplification curve had a Ct that is 4 cycles less than the other clone. The other clone appears to have a single copy of the heavy chain YAC. The clone containing one copy of each YAC (clone SC) is selected for further analysis.

PCR primers are designed to amplify 300-500 bp regions of each YAC which are complementary to restriction fragments to be detected in the Southern blot analysis. PCR products are gel purified and quantitated by the Picogreen Assay (Molecular Probes). Radiolabeled probes are generated by random priming using deoxycytidine 5′-[a-32P] triphosphate and the Rediprime II Random Priming kit (Amersham).

Cells of clone SC are embedded in agarose plugs and subjected to DNA release and restriction digestion according to standard protocols. Several enzymes that cut the YACs into 20 to 150 kb segments are used including Asc I, Pac I and Sbf I. The digested plugs are loaded in multiple lanes such that replicate membranes can be cut from a single membrane. The digested DNAs are separated by PFGE (CHEF) on a 0.8% agarose gel in TAE buffer (switch time 1=1 s, switch time 2=25 s, 4 V/cm, 15 to 20 h, 14° C.). The gel is transferred to a UV crosslinker (Stratagene) and exposed to 120 mJ UV radiation. The gel is denatured in 1.5 M NaCl, 0.5 M NaOH for 30 minutes at RT and neutralized in 1.5 M NaCl, 1.0 M Tris base, pH 7.4 for 40 minutes at RT. The gel is transferred by capillary action to Genescreen Plus® nylon membrane in 10×SSPE for one to three days. The membrane is briefly rinsed in 2×SSPE and cross-linked with 120 mJ UV radiation (Stratagene). The membrane is cut into replicate pieces and is transferred to roller bottles (Bellco). The membranes are prehybridized in hybridization buffer (1.25×SSPE, 0.625% SDS, 40% formamide, 1×Denhardts, 10% dextran sulfate, 0.05 mg/ml denatured salmon sperm DNA) for 2-6 hours at 42° C. The hybridization buffer is changed with new buffer and the appropriate probe is added. The membranes are hybridized overnight at 42° C. The next day the membranes are washed with 0.2×SSPE, 1% SDS or 0.02×SSPE, 1% SDS at 42° C. to 65° C. until the CPM of each membrane is 400 or less. Membranes are wrapped in Saran Wrap® and exposed one to three days to BioMax MST™ film with a BioMax TranScreen HE™ intensifying screen at −80° C. Clone SC is found to have restriction fragments which demonstrate the structural integrity of both YACs; i.e., no rearrangement of the YACs is apparent.

### Example 29

### Purification of Ig Loci Containing Artificial Chromosome and Analysis of Human Immunoglobulin Produced in Transgenic Avians

Artificial chromosomes are purified from clone SC by flow cytometry and are used for cytoplasmic injections of stage I White leghorn embryos essentially as disclosed in Example 21. 500 embryos are injected with between 100 and 1000 artificial chromosomes. 135 chicks hatch and are analyzed for the presence of the transgene in their blood DNA. DNA is extracted as disclosed in U.S. Pat. No. 6,423,488, issued Jul. 23, 2002. 100 ng of DNA is analyzed by real-time PCR using probes to detect the heavy and light chain YACs as disclosed in Example 31. Five birds are found to be positive for the clone SC artificial chromosome at significant levels (>1 copy of the artificial chromosome for every 100 genomic equivalents).

Serum from hatched birds and eggs from mature hens are analyzed for human Igλ and IgFc levels by ELISA. Several birds are positive for both human Igλ and IgFc in their serum, indicating that human IgG is produced in the serum. Eggs from G0 hens are collected and the yolks removed. Yolk is diluted and analyzed for human Igλ and IgFc levels by ELISA. Several hens contain human IgG in the yolk of their eggs.

G1 birds are produced from the G0 birds as disclosed herein. Each of the positive G1 birds include the artificial chromosome in substantially all of their somatic cells as demonstrated by FISH. The germline transgenic G1 birds produce substantial quantities of polyclonal antibodies which are deposited in the egg. For example, human polyclonal antibody is present in an amount greater than about 10 μg/egg or greater than about 0.1 mg/egg.

### Example 30

### Isolation and Characterization of Human Immunoglobulin Loci YACs

Two YACs that contain substantial portions of the human light-chain and heavy-chain immunoglobulin loci are shown in FIGS. 27A and 27B. These constructs contain multiple variable, D, J and constant regions, as well as elements required for gene expression, gene rearrangement and constant chain switch. The lambda light-chain construct, IgLambda, is a 410 kb YAC that has been previously used to express human polyclonal antibodies in transgenic mice. See, for example, US patent application No. 2004/0231012, published Nov. 18, 2004 and Popov et al (1999) J. Exp. Med. 189:1611-1619, the disclosures of which are incorporated in their entireties herein by reference. The heavy-chain construct, IgHeavy-2, is a 300 kb derivative of the YAC shown in FIG. 27A that has been used to express human polyclonal in mice (Nicholson et al (1999) J Immunol 163:6898-6906) to which a functional human gamma-constant gene segment has been added 3′ of the Cδ region.

YAC containing strains of Saccharomyces cerevisiae were grown in a yeast nitrogen base medium with 2% glucose and an appropriate selective amino acid at 30° C. for 4 days. Total DNA agarose plugs were prepared from the yeast strains using the protocol of Iadonato, S. P., and A. Gnirke (1996) modified as follows:

Yeast cells were centrifuged, washed with 50 mM EDTA pH 8 and resuspended at 2×10⁹ cells/ml in 50 mM EDTA pH 8. The cell suspension was heated to 45-50° C. and added to an equal volume of 2% LMP agarose that had been melted and brought to 45-50° C. Cells and agarose were mixed and dispensed into plug molds which were then placed at 4° C. Hardened plugs were placed in spheroplasting solution (1 M sorbital, 20 mM EDTA, 10 mM Tris-HCl pH 7.5, 14 mM mercaptoethanol, 3% lyticase solution (#170-3593 Bio-Rad)) at 37° C. for 4 hours with gentle agitation. Plugs were then washed in LDS solution (1% lithium dodecyl sulfate, 100 mM EDTA pH 8, 10 mM Tris-HCl pH 8) for 15 minutes and were then placed in LDS solution for 16 hours at 37° C. with gentle agitation. Plugs were then washed 3 times for 30 minutes in NDS solution (500 mM EDTA, 10 mM Tris base, 1% sarkosyl pH 9) and 5 times for 30 minutes with TE (10 mM Tris-HCl pH 8, 1 mM EDTA pH 8) with gentle agitation.

The intact YACs were separated by contour-clamped homogeneous electric field (CHEF) electrophoresis in 1% low-melting point agarose gels using 0.5×TBE buffer at 14° C. and a 30 second constant switch time at 5 V/cm for 36 hr. Gel slices containing the YAC of interest were equilibrated 2 hr with microinjection buffer containing 10 mM Tris-Cl pH 7.5, 0.1 mM EDTA pH 8.0, 100 mM NaCl, 30 mM spermine, and 70 mM spermidine. The gel slices were melted at 68° C. for 20 min and then digested with GELase (5 U/100 mg) at 42° C. for 2 hr. Integrity of Each YAC sample was then confirmed by CHEF electrophoresis on a 1.5% agarose gel with 0.5×TBE buffer at 14° C. using a 30 second constant switch time and 5 V/cm for 24 hr.

### Example 31

### Transgenesis and Immunoglobulin Expression

Purified heavy-chain and light-chain YAC DNAs prepared as disclosed in Example 30 were co-injected into early embryos to generate transgenic animals as essentially disclosed in Example 21. A volume of 50 nl of 110 pg chromosome DNA per μl of microinjection buffer was injected into each of several hundred embryos. Testing for the production of human light-chain in serum of resultant chickens was performed using a human lambda ELISA quantitation kit (#E80-116) from Bethyl Laboratories (Montgomery, Tex.). In the procedure, both the capture antibody and detection antibodies were diluted 1:2000. Quantitation of antibody containing associated light-chain and heavy-chains was performed by replacing the detection antibody in the above kit with an alkaline phosphatase-conjugated goat anti-human IgG, Fc gamma-antibody (diluted 1:2000) (#109-056-098, Jackson ImmunoResearch Laboratories, Inc., West Grove, Pa.) and followed by detection using a TMB substrate. At least one bird was shown to express human immunoglobulins by ELISA (Table 5) in the serum.

TABLE 5

| Bird | Lambda Light-Chain | Whole IgGλ |
|---|---|---|
| #6946 | 26 ng/ml | 24 ng/ml |
| Control | 0 | 0 |

### Example 32

### Identification of Target Sequences and Probe Preparation

A 234 bp sequence of mouse major satellite DNA sequence (SEQ ID NO: 14) present in SATACs (see, for example, US patent publication No. 2003/0119104, filed May 30, 2002, the disclosure of which is incorporated in its entirety herein by reference) was scanned for 6mer repeat regions. Two such sequences were identified, each present five times in the 234 bp sequence. These consensus sequences can be targets for labeled polyamide probes. The identified sequences are shown in Table 6.

Mouse Major Satellite DNA sequence

SEQ ID NO: 14

gacctggaatatcgcgagtaaactgaaaatcacggaaaatgagaaataca
cactttaggacgtgaaatatggcgaggaaaactgaaaaaggtggaaaatt
tagaaatgtccactgtaggacgtggaatatggcaagaaaactgaaaatca
tggaaaatgagaaacatccacttgacgacttgaaaaatgacgaaatcact
aaaaaacgtgaaaaatgagaaatgcacactgaaggac,

TABLE 6

| | | |
|---|---|---|
| Pattern Searched | Consensus | Copy No |

| | | |
|---|---|---|
| TGAAAA | TGAAAA | 5 |
| GAAAAT | GAAAAT | 5 |

The centromeric region of SATACs contain copies of the mouse minor satellite sequence which can also be targeted. For example, polyamide probes can be designed to target pentameric repeat sequences and octameric repeat sequences located within the 120 bp sequence of the mouse minor satellite (SEQ ID NO: 15). The consensus or repeat sequences identified are shown in Table 7.

Mouse minor satellite DNA Sequence

SEQ ID NO: 15

gagtgagttacactgaaaaacacatacgttggaaaccggcattgtagaac
agtgtatatcaatgagttacaatgagaaaaatggaaaatgataaaaacca
cagtgtagaacatattagatgtgtgagttacactgaaaaacacattcctt
ggaaacgggatttgtagaactgtgtatatcaatgagttacaatgagaaac
atggaaaatgataaaaaccacactgtagaacattttagatgagtgagtta
cactgaaaaacacatatgttggaaa,

TABLE 7

| | | |
|---|---|---|
| Pattern Searched | Consensus | Copy No |

| | | |
|---|---|---|
| GAAAA | GAAAA | 6 |
| AAAAA | AAAAA | 6 |
| AATGA | AATGA | 6 |
| TGAGTTAC | TGAGTTAC | 5 |
| GAGTTACA | GAGTTACA | 5 |

The heterochromatic region of SATACs contain copies of the mouse rDNA (ribosomal RNA encoding DNA) sequence can also be targeted. For example, polyamide probes can be designed to target 5-mer repeat sequences, six-mer repeat sequences and seven-mer repeat sequences located within the 120 bp sequence of mouse rDNA shown in SEQ ID NO. 16. The consensus or repeat sequences are shown in Table 8.

TABLE 8

| | | |
|---|---|---|
| Pattern Searched | Consensus | Copy No |

| | | |
|---|---|---|
| TGTGC | TGTGC | 6 |
| TTCCC | TTCCC | 6 |
| CGTGC | CGTGC | 8 |
| CCGCC | CCGCC | 21 |
| CGCCG | CGCCG | 25 |
| CCCGCG | CCCGCG | 15 |
| CCCGTC | CCCGTC | 5 |
| CCGGCG | CCGGCG | 7 |
| CCCGGG | CCCGGG | 5 |
| TCTCTCG | TCTCTCG | 6 |

Polyamide probes can be constructed to recognize the repeat sequences shown in Tables 6, 7 and 8 and/or other sequences contained in artificial chromosomes that will provide for a facilitated isolation of the artificial chromosomes, for example, by flow cytometry. Methods of making polyamide probes are well known in the art and are disclosed, for example, in the certain references cited herein and in the specification.

### Example 33

### Preparation and Flow Sorting of Chromosomes

Chromosome suspensions are prepared from CHO cells (e.g., chromosome suspensions containing artificial chromosomes such as those disclosed in US patent publication No. 2003/0119104, filed May 30, 2002) using a modification of the polyamine-based method described by Sillar and Young (1981) A new method for the preparation of metaphase chromosomes for flow analysis, J. Histochem. Cytochem., 29, 74-78 and Lalande et al (1984) Development and use of metaphase chromosome flow-sorting methodology to obtain recombinant phage libraries enriched for parts of the human X chromosome, Cytometry, 5, 101-107, the disclosures of which are incorporated herein in their entirety by reference. Briefly, cells cultured in RPMI medium containing 20% fetal bovine serum are arrested at mitosis by incubation in 0.1 μg/ml colcemid for about 16 h. The cells are collected by centrifugation, resuspended in 40 mM KCl for 10 min and then centrifuged again. The pellet is resuspended in cold buffer containing 80 mM KCl, 20 mM NaCl, 15 mM Tris-HCl pH 7.2, 2 mM EDTA, 0.5 mM EGTA, 7 mM β-mercaptoethanol, 0.2 mM spermine, 0.5 mM spermidine and 0.12% digitonin, and incubated on ice for 10 min. The suspension is vortexed vigorously for 2 min and then stored for up to 90 days at 4° C. before use.

Prior to flow analysis, the chromosomes are stained for about 2 h with 1 μM of fluorescently labeled polyamide probe and 2 μg/ml HO (Hoechst 33258). The polyamide probe targets one or more of the nucleotide sequences specified in Table 6, Table 7 and/or Table 8 and is produced essentially as disclosed in Dervan (2001) Molecular recognition of DNA by small molecules. Bioorg Med Chem 9: 2215-35.

Sodium citrate and sodium sulfite are added to chromosomes 15 to 30 min before flow analysis at final concentrations of 10 and 25 mM, respectively, to improve chromosome resolution.

Chromosomes are separated using an Influx flow sorter (Cytopeia, Inc., Seattle, Wash.). One laser is tuned to emit ultraviolet light (351-364 nm, 250 mW) to excite HO, and HO fluorescence is measured after passing through a 425-nm long-pass filter and a 458-nm rejection-band filter. A second laser is tuned to 458 nm (250 mW) to excite the fluorescently labeled polyamide probe. The fluorescently labeled polyamide probe fluorescence is measured after passing through a 500-nm long-pass filter and a 458-nm rejection-band filter. Alternatively, fluorescein fluorescence is measured following excitation at 488 nm (250 mW) after passing through a 530/40 band-pass filter. The fluorescence pulses from the individual chromosomes are integrated by a data acquisition system, and are collected in listmode at a rate of about 1000 chromosomes per second.

### Example 34

### Purification of SATACs Contained in Micronuclei

Micronucleation of chromosomes in ChY1 cells containing the artificial chromosome (e.g., SATACs) is induced by incubation for 72 h in the presence of 1 μg/ml of colchicine in growth medium.

Micronuclei are isolated essentially as described in Labidi (Labidi, B, et al. Procedure for isolating micronuclei from rat kangaroo cultured cells containing individualized chromosomes. Eur J Cell Biol 38: 165-70, 1985), the disclosure of which is incorporated in its entirety herein by reference. Briefly, micronucleated cells are harvested by trypsin-EDTA treatment, rinsed twice in PBS, and resuspended in 2 vol TKM buffer (10 mM Tris-HCl, pH 7.4, 10 mM KCl, and 3 mM MgCl₂) containing 0.05% collagenase 1 A (Sigma). Cell lysis is accelerated by gentle shearing of the suspension through a 26-gauge needle and 1 mM phenyl methyl sulfonyl fluoride (PMSF) is added. Isolated micronuclei are collected by low-speed centrifugation (1500 g) and washed twice with 4 vol of Tris-polyamine buffer (TPB; 15 mM Tris-HCl, pH 7.4, 0.2 mM spermine, 0.5 mM spermidine, 2 mM EDTA, 0.5 mM EGTA, 80 mM KCl, 20 mM NaCl, and 14 mM B-mercaptoethanol).

The micronuclei containing chromosomes are stained and purified by flow cytometry in essentially the same manner as described for the staining and flow cytometry purification of artificial chromosomes, as disclosed in Example 33.

During the flow cytometry, micronuclei collection is limited to the window in the fluorescence histogram where micronuclei containing a single SATAC are located, which can be defined by conventional methodologies.

### Example 35

### Production of Transchromosomic Chickens Using Satellite DNA-Based Artificial Chromosomes

The flow-sorted artificial chromosomes of Example 33 or micronuclei containing the artificial chromosomes of Example 34 are pelleted by centrifugation of a 750 μl sample containing approximately 10⁶ chromosomes (artificial chromosomes or micronuclei containing artificial chromosomes) at 2500×g for 30 min at 4° C. The supernatant, except the bottom 30 microliters (μl) containing the chromosomes, is removed resulting in a concentration of about 7000 to 11,500 chromosomes per μl of injection buffer (Monteith, et al. Methods Mol Biol 240: 227-242, 2004). Approximately 25 to 100 nanoliters (nl) of injection buffer is injected per embryo.

Early stage embryos (e.g., stage I embryos) are collected from 24 to 36 week-old hens from commercial White Leghorn variety of G. gallus. Embryo donor hens are inseminated weekly using pooled semen from roosters of the same breed to produce eggs for injection.

On the day of egg collection, fertile hens are euthanized 2 h post oviposition by cervical dislocation. Typically, oviposition is followed by ovulation of the next egg after about 24 minutes (Morris, Poultry Science 52: 423-445, 1973). The recently ovulated and fertilized eggs are collected from the upper magnum region of the oviduct under sterile conditions and placed in a glass well and covered with Ringers'Medium (Tanaka, et al. J Reprod Fertil 100: 447-449, 1994) and maintained at 41° C. until microinjection.

Cytoplasmic injection of artificial chromosomes is achieved using the microinjection apparatus disclosed in U.S. patent application Ser. No. 11/159,973, filed Jun. 23, 2005, the disclosure of which is incorporated in its entirety herein by reference. Chromosomes are injected into the Stage I embryos at a single site. Each embryo is cytoplasmically injected with approximately 400 to 1000 chromosomes. The chromosomes are injected in a suspension of 25 to 100 nanoliters (nl) of injection buffer.

Following microinjection, the embryos are transferred to the oviduct of recipient hens using the ovum transfer (OT) procedure of Olsen (Olsen, M and Neher, B. J Exp Zool 109: 355-66, 1948), with the exception that the hens are anesthetized by isofluorane gas. Typically, about 26 h after OT, the recipient hens lay a hard shell egg containing the manipulated ovum. Eggs are incubated for 21 days in a regular incubator until hatching of the birds.

Transchromosomic founders are identified based on PCR analysis and FISH analysis. Analysis of G1 embryos from a test bird show the artificial chromosome to be transmitted through the germline.

While this invention has been described with respect to various specific examples and embodiments, it is to be understood that the invention is not limited thereto and that it can be variously practiced with the scope of the following claims.

What is claimed is:

1. A method comprising:
isolating an artificial chromosome containing a centromere;
injecting the artificial chromosome containing a centromere into an avian embryo;
maintaining the embryo under conditions suitable for the embryo to develop and hatch as a chick; and
maintaining the chick under conditions suitable to obtain an avian wherein the artificial chromosome containing a centromere is present in the genome of the avian.

2. The method of claim 1 wherein the artificial chromosome contains a nucleotide sequence which can function as a marker.

3. The method of claim 1 wherein the artificial chromosome contains heterochromatic DNA which can function as a marker.

4. The method of claim 1 wherein the artificial chromosome is isolated by flow cytometry.

5. The method of claim 4 wherein the flow cytometry is facilitated by a probe which is associated with the artificial chromosome.

6. The method of claim 1 wherein the artificial chromosome is introduced into the avian embryo by injection.

7. The method of claim 1 further comprising transferring the embryo to a recipient female avian.

8. The method of claim 1 wherein the embryo is an early stage embryo.

9. The method of claim 1 wherein the embryo is a stage I embryo.

10. The method of claim 1 wherein the avian is a chicken.

11. The method of claim 1 wherein the artificial chromosome comprises a heterologous recombination site.

12. The method of claim 11 wherein the artificial chromosome comprises more than one heterologous recombination site.

13. The method of claim 1 wherein the artificial chromosome comprises a heterologous coding sequence.

14. The method of claim 13 wherein the heterologous coding sequence comprises a pharmaceutical protein coding sequence.

15. The method of claim 13 wherein the heterologous coding sequence encodes a cytokine.

16. The method of claim 13 wherein the heterologous coding sequence encodes a pharmaceutical protein selected from the group consisting of antibody and enzyme.

17. The method of claim 1 wherein the artificial chromosome comprises a promoter.

18. The method of claim 1 wherein the artificial chromosome comprises a promoter which functions in tubular gland cells.

19. The method of claim 1 wherein the artificial chromosome comprises an IRES.

20. The method of claim 1 further comprising obtaining an offspring from the avian wherein the offspring contains an artificial chromosome in its genome.

21. An avian produced according to the method of claim 1.

22. A chicken produced according to the method of claim 1.


SEQUENCE LISTING (16 sequences)

SEQ ID NO: 1 - DNA, 6230 bp, Artificial Sequence
  Plasmid pCMV-31int
cattcgccattcaggctgcgcaactgttgggaagggcgatcggtgcgggcctcttcgctattacgccagccaatacgcaaaccgcctctccccgcgcgttggccgattcattaatgcaggatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcatgaacagactgtgaggactgaggggcctgaaatgagccttgggactgtgaatctaaaatacacaaacaattagaatcactagctcctgtgtataatattttcataaatcatactcagtaagcaaaactctcaagcagcaagcatatgcagctagtttaacacattatacacttaaaaattttatatttaccttagagctttaaatctctgtaggtagtttgtccaattatgtcacaccacagaagtaaggttccttcacaaagatcccaagctagcttataatacgactcactatagggagagagctatgacgtcgcatgcacgcgtaagcttgggcccctcgagggatccgggtgtctcgctacgccgctacgtcttccgtgccgtcctgggcgtcgtcttcgtcgtcgtcggtcggcggcttcgcccacgtgatcgaagcgcgcttctcgatgggcgttccctgccccctgcccgtagtcgacttcgtgacaacgatcttgtctacgaagagcccgacgaacacgcgcttgtcgtctactgacgcgcgcccccaccacgacttagggccggtcgggtcagcgtcggcgtcttcggggaaccattggtcaaggggaagcttcggggcttcggcggcttcaagttcggcaagccgctcttccgccccttgctgccggagcgtcagcgctgcctgttgcttccggaagtgcttcctgccaacgggtccgtcgtacgcgcctgccgcgcggtcttcgtacagctcttcaagggcgttcagggcgtcggcgcgctccgcaacaaggttcgcccgttcgccgctcttctcaggcgcctcagtgagcttgccgaagcgtcgggcggcttcccacagaagcgccaacgtctcttcgtcgccttcggcgtgcctgatcttgttgaagatgcgttccgcaacgaacttgtcgagtgccgccatgctgacgttgcacgtgccttcgtgctgcccaggtgcggacgggtcgaccaccttccggcgacggcagcggtaagagtccttgatcgattcttccccgcgcttcgaagtcatgacggcgccacactcgcagtacagcttgtccatggcggacagaatggcttgcccccgggaaagccccttgccgcgccccctgccgtccaaccacgcctgaagctcataccactcagcgggctcgatgatcggtccgcaatcaagctcgaccggccggagcgtgatcgggtcgcgctgaatgcggtaaccctcaatcttcgtggtcggcgtgccgtccggcttcttcttgtagatcacctcagcggcgaagcccgcaatacgcgggtcccgaaggattcgcataacggttgccgggtcccaggcgcttgaagcggtcttcttcccaatcgtctcgccccgggtcggcacggcgtcagcgtccatgcgcttacaaagccccgtgatgctgcccgggtgaatggcggcttgactgcccggcttgaagggaaggtgtttgtgcgtcttgatctcacgccaccaccaccggattacgtcgggctcgaactcgaagggtccggtaaggggagtggtcgagtgcgcaagcttgttgatgacgacattgaccattcggccgttgcgcgtgatctccttcgtctccgaaacaagctcgaagccgtaaggcgccttcccgccgacgtacccgcccaattcgcgctgaaggttcttcgtgtcgagaatcttcgccgacttcagcgaagattctttgtgcgacgcgtcgagccgcataatcaggtgaatcaggtccatgacgtttccctgccggaagacgccttcctgagtggaaacaatcgtcacgcccagggcgagcaattccgagacaatcggaatcgcgtccatgaccttcaggcgcgagaagcgcgacacgtcatagacaatgatcatgttgagccgcccggcgcggcattcgttcaggatgcgttcgaactccgggcgctccgccgtcccgaacgccgacgtgcccggcgcttcgctgaaatgcccgacgaacctgaaccggcccccgtcgcgctcgacttcgcgctgaaggtcggccgccttgtcttcgttggcgctacgctgtgtcgctgggcttgctgcgctcgaattctcgcgctcgcgcgactgacggtcgtaagcacccgcgtacgtgtccaccccggtcacaaccccttgtgtcatgtcggcgaccctacgactagtgagctcgtcgacccgggaattccggaccggtacctgcaggcgtaccttctatagtgtcacctaaatagctttttgcaaaagcctaggctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgacctgcacgatgctgtttcctgtgtgaaattgttatccgctcacaattccacacattatacgagccggaagctataaagtgtaaagcctggggtgcctaatgagtgaaagggcctcgtatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaacgcgcgaattgcaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccataacttcgtatagcatacattatacgaagttatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgccaggggtgggcacacatatttgataccagcgatccctacacagcacataattcaatgcgacttccctctatcgcacatcttagacctttattctccctccagcacacatcgaagctgccgagcaagccgttctcaccagtccaagacctggcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 2 - DNA, 5982 bp, Artificial Sequence
  Plasmid pCMV-luc-attB
ctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcaggtaccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacattt

SEQ ID NO: 3 - DNA, 5924 bp, Artificial Sequence
  Plasmid pCMV-luc-attP
ctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcgactagtactgacggacacaccgaagccccggcggcaaccctcagcggatgccccggggcttcacgttttcccaggtcagaagcggttttcgggagtagtgccccaactggggtaacctttgagttctctcagttgggggcgtagggtcgccgacatgacacaaggggttgtgaccggggtggacacgtacgcgggtgcttacgaccgtcagtcgcgcgagcgcgactagtacaagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacattt

SEQ ID NO: 4 - DNA, 5101 bp, Artificial Sequence
  Plasmid pCMV-pur-attB
ctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcaggtaccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatctgcgatctaagtaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgact

SEQ ID NO: 5 - DNA, 5043 bp, Artificial Sequence
  Plasmid pCMV-pur-attP
ctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcgactagtactgacggacacaccgaagccccggcggcaaccctcagcggatgccccggggcttcacgttttcccaggtcagaagcggttttcgggagtagtgccccaactggggtaacctttgagttctctcagttgggggcgtagggtcgccgacatgacacaaggggttgtgaccggggtggacacgtacgcgggtgcttacgaccgtcagtcgcgcgagcgcgactagtacaagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatctgcgatctaagtaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgact

SEQ ID NO: 6 - DNA, 5041 bp, Artificial Sequence
  Plasmid pCMV-EGFP-attB
ctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcaggtaccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctcgagcatgcat

SEQ ID NO: 7 - DNA, 18116 bp, Artificial Sequence
  Plasmid p12.0lys-LSPIPNMM-CMV-pur-attB
gggctgcaggaattcgattgccgccttctttgatattcactctgttgtatttcatctcttcttgccgatgaaaggatataacagtctgtataacagtctgtgaggaaatacttggtatttcttctgatcagtgtttttataagtaatgttgaatattggataaggctgtgtgtcctttgtcttgggagacaaagcccacagcaggtggtggttggggtggtggcagctcagtgacaggagaggtttttttgcctgtttttttttttttttttttttttaagtaaggtgttcttttttcttagtaaattttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacacgagataccttattaagtttaggccagcttgatgctttattttttccctttgaagtagtgagcgttctctggtttttttcctttgaaactggtgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttcctttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttggctgctgccttcccgagatccaggacactaaactgcttctgcactgaggtataaatcgcttcagatcccagggaagtgcagatccacgtgcatattcttaaagaagaatgaatactttctaaaatattttggcataggaagcaagctgcatggatttgtttgggacttaaattattttggtaacggagtgcataggttttaaacacagttgcagcatgctaacgagtcacagcgtttatgcagaagtgatgcctggatgcctgttgcagctgtttacggcactgccttgcagtgagcattgcagataggggtggggtgctttgtgtcgtgttcccacacgctgccacacagccacctcccggaacacatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaaagcttctttggaaatacactgacttgattgaagtctcttgaagatagtaaacagtacttacctttgatcccaatgaaatcgagcatttcagttgtaaaagaattccgcctattcataccatgtaatgtaattttacacccccagtgctgacactttggaatatattcaagtaatagactttggcctcaccctcttgtgtactgtattttgtaatagaaaatattttaaactgtgcatatgattattacattatgaaagagacattctgctgatcttcaaatgtaagaaaatgaggagtgcgtgtgcttttataaatacaagtgattgcaaattagtgcaggtgtccttaaaaaaaaaaaaaaaaagtaatataaaaaggaccaggtgttttacaagtgaaatacattcctatttggtaaacagttacatttttatgaagattaccagcgctgctgactttctaaacataaggctgtattgtcttcctgtaccattgcatttcctcattcccaatttgcacaaggatgtctgggtaaactattcaagaaatggctttgaaatacagcatgggagcttgtctgagttggaatgcagagttgcactgcaaaatgtcaggaaatggatgtctctcagaatgcccaactccaaaggattttatatgtgtatatagtaagcagtttcctgattccagcaggccaaagagtctgctgaatgttgtgttgccggagacctgtatttctcaacaaggtaagatggtatcctagcaactgcggattttaatacattttcagcagaagtacttagttaatctctacctttagggatcgtttcatcatttttagatgttatacttgaaatactgcataacttttagctttcatgggttcctttttttcagcctttaggagactgttaagcaatttgctgtccaacttttgtgttggtcttaaactgcaatagtagtttaccttgtattgaagaaataaagaccatttttatattaaaaaatacttttgtctgtcttcattttgacttgtctgatatccttgcagtgcccattatgtcagttctgtcagatattcagacatcaaaacttaacgtgagctcagtggagttacagctgcggttttgatgctgttattatttctgaaactagaaatgatgttgtcttcatctgctcatcaaacacttcatgcagagtgtaaggctagtgagaaatgcatacatttattgatacttttttaaagtcaactttttatcagatttttttttcatttggaaatatattgttttctagactgcatagcttctgaatctgaaatgcagtctgattggcatgaagaagcacagcactcttcatcttacttaaacttcattttggaatgaaggaagttaagcaagggcacaggtccatgaaatagagacagtgcgctcaggagaaagtgaacctggatttctttggctagtgttctaaatctgtagtgaggaaagtaacacccgattccttgaaagggctccagctttaatgcttccaaattgaaggtggcaggcaacttggccactggttatttactgcattatgtctcagtttcgcagctaacctggcttctccactattgagcatggactatagcctggcttcagaggccaggtgaaggttgggatgggtggaaggagtgctgggctgtggctggggggactgtggggactccaagctgagcttggggtgggcagcacagggaaaagtgtgggtaactatttttaagtactgtgttgcaaacgtctcatctgcaaatacgtagggtgtgtactctcgaagattaacagtgtgggttcagtaatatatggatgaattcacagtggaagcattcaagggtagatcatctaacgacaccagatcatcaagctatgattggaagcggtatcagaagagcgaggaaggtaagcagtcttcatatgttttccctccacgtaaagcagtctgggaaagtagcaccccttgagcagagacaaggaaataattcaggagcatgtgctaggagaactttcttgctgaattctacttgcaagagctttgatgcctggcttctggtgccttctgcagcacctgcaaggcccagagcctgtggtgagctggagggaaagattctgctcaagtccaagcttcagcaggtcattgtctttgcttcttcccccagcactgtgcagcagagtggaactgatgtcgaagcctcctgtccactacctgttgctgcaggcagactgctctcagaaaaagagagctaactctatgccatagtctgaaggtaaaatgggttttaaaaaagaaaacacaaaggcaaaaccggctgccccatgagaagaaagcagtggtaaacatggtagaaaaggtgcagaagcccccaggcagtgtgacaggcccctcctgccacctagaggcgggaacaagcttccctgcctagggctctgcccgcgaagtgcgtgtttctttggtgggttttgtttggcgtttggttttgagatttagacacaagggaagcctgaaaggaggtgttgggcactattttggtttgtaaagcctgtacttcaaatatatattttgtgagggagtgtagcgaattggccaatttaaaataaagttgcaagagattgaaggctgagtagttgagagggtaacacgtttaatgagatcttctgaaactactgcttctaaacacttgtttgagtggtgagaccttggataggtgagtgctcttgttacatgtctgatgcacttgcttgtccttttccatccacatccatgcattccacatccacgcatttgtcacttatcccatatctgtcatatctgacatacctgtctcttcgtcacttggtcagaagaaacagatgtgataatccccagccgccccaagtttgagaagatggcagttgcttctttccctttttcctgctaagtaaggattttctcctggctttgacacctcacgaaatagtcttcctgccttacattctgggcattatttcaaatatctttggagtgcgctgctctcaagtttgtgtcttcctactcttagagtgaatgctcttagagtgaaagagaaggaagagaagatgttggccgcagttctctgatgaacacacctctgaataatggccaaaggtgggtgggtttctctgaggaacgggcagcgtttgcctctgaaagcaaggagctctgcggagttgcagttattttgcaactgatggtggaactggtgcttaaagcagattccctaggttccctgctacttcttttccttcttggcagtcagtttatttctgacagacaaacagccacccccactgcaggcttagaaagtatgtggctctgcctgggtgtgttacagctctgccctggtgaaaggggattaaaacgggcaccattcatcccaaacaggatcctcattcatggatcaagctgtaaggaacttgggctccaacctcaaaacattaattggagtacgaatgtaattaaaactgcattctcgcattcctaagtcatttagtctggactctgcagcatgtaggtcggcagctcccactttctcaaagaccactgatggaggagtagtaaaaatggagaccgattcagaacaaccaacggagtgttgccgaagaaactgatggaaataatgcatgaattgtgtggtggacattttttttaaatacataaactacttcaaatgaggtcggagaaggtcagtgttttattagcagccataaaaccaggtgagcgagtaccatttttctctacaagaaaaacgattctgagctctgcgtaagtataagttctccatagcggctgaagctcccccctggctgcctgccatctcagctggagtgcagtgccatttccttggggtttctctcacagcagtaatgggacaatacttcacaaaaattctttcttttcctgtcatgtgggatccctactgtgccctcctggttttacgttaccccctgactgttccattcagcggtttggaaagagaaaaagaatttggaaataaaacatgtctacgttatcacctcctccagcattttggtttttaattatgtcaataactggcttagatttggaaatgagagggggttgggtgtattaccgaggaacaaaggaaggcttatataaactcaagtcttttatttagagaactggcaagctgtcaaaaacaaaaaggccttaccaccaaattaagtgaatagccgctatagccagcagggccagcacgagggatggtgcactgctggcactatgccacggcctgcttgtgactctgagagcaactgctttggaaatgacagcacttggtgcaatttcctttgtttcagaatgcgtagagcgtgtgcttggcgacagtttttctagttaggccacttcttttttccttctctcctcattctcctaagcatgtctccatgctggtaatcccagtcaagtgaacgttcaaacaatgaatccatcactgtaggattctcgtggtgatcaaatctttgtgtgaggtctataaaatatggaagcttatttatttttcgttcttccatatcagtcttctctatgacaattcacatccaccacagcaaattaaaggtgaaggaggctggtgggatgaagagggtcttctagctttacgttcttccttgcaaggccacaggaaaatgctgagagctgtagaatacagcctggggtaagaagttcagtctcctgctgggacagctaaccgcatcttataaccccttctgagactcatcttaggaccaaatagggtctatctggggtttttgttcctgctgttcctcctggaaggctatctcactatttcactgctcccacggttacaaaccaaagatacagcctgaattttttctaggccacattacataaatttgacctggtaccaatattgttctctatatagttatttccttccccactgtgtttaaccccttaaggcattcagaacaactagaatcatagaatggtttggattggaaggggccttaaacatcatccatttccaaccctctgccatgggctgcttgccacccactggctcaggctgcccagggccccatccagcctggccttgagcacctccagggatggggcacccacagcttctctgggcagcctgtgccaacacctcaccactctctgggtaaagaattctcttttaacatctaatctaaatctcttctcttttagtttaaagccattcctctttttcccgttgctatctgtccaagaaatgtgtattggtctccctcctgcttataagcaggaagtactggaaggctgcagtgaggtctccccacagccttctcttctccaggctgaacaagcccagctccttcagcctgtcttcgtaggagatcatcttagtggccctcctctggacccattccaacagttccacggctttcttgtggagccccaggtctggatgcagtacttcagatggggccttacaaaggcagagcagatggggacaatcgcttacccctccctgctggctgcccctgttttgatgcagcccagggtactgttggcctttcaggctcccagaccccttgctgatttgtgtcaagcttttcatccaccagaacccacgcttcctggttaatacttctgccctcacttctgtaagcttgtttcaggagacttccattctttaggacagactgtgttacacctacctgccctattcttgcatatatacatttcagttcatgtttcctgtaacaggacagaatatgtattcctctaacaaaaatacatgcagaattcctagtgccatctcagtagggttttcatggcagtattagcacatagtcaatttgctgcaagtaccttccaagctgcggcctcccataaatcctgtatttgggatcagttaccttttggggtaagcttttgtatctgcagagaccctgggggttctgatgtgcttcagctctgctctgttctgactgcaccattttctagatcacccagttgttcctgtacaacttccttgtcctccatcctttcccagcttgtatctttgacaaatacaggcctatttttgtgtttgcttcagcagccatttaattcttcagtgtcatcttgttctgttgatgccactggaacaggattttcagcagtcttgcaaagaacatctagctgaaaactttctgccattcaatattcttaccagttcttcttgtttgaggtgagccataaattactagaacttcgtcactgacaagtttatgcattttattacttctattatgtacttactttgacataacacagacacgcacatattttgctgggatttccacagtgtctctgtgtccttcacatggttttactgtcatacttccgttataaccttggcaatctgcccagctgcccatcacaagaaaagagattccttttttattacttctcttcagccaataaacaaaatgtgagaagcccaaacaagaacttgtggggcaggctgccatcaagggagagacagctgaagggttgtgtagctcaatagaattaagaaataataaagctgtgtcagacagttttgcctgatttatacaggcacgccccaagccagagaggctgtctgccaaggccaccttgcagtccttggtttgtaagataagtcataggtaacttttctggtgaattgcgtggagaatcatgatggcagttcttgctgtttactatggtaagatgctaaaataggagacagcaaagtaacacttgctgctgtaggtgctctgctatccagacagcgatggcactcgcacaccaagatgagggatgctcccagctgacggatgctggggcagtaacagtgggtcccatgctgcctgctcattagcatcacctcagccctcaccagcccatcagaaggatcatcccaagctgaggaaagttgctcatcttcttcacatcatcaaacctttggcctgactgatgcctcccggatgcttaaatgtggtcactgacatctttatttttctatgatttcaagtcagaacctccggatcaggagggaacacatagtgggaatgtaccctcagctccaaggccagatcttccttcaatgatcatgcatgctacttaggaaggtgtgtgtgtgtgaatgtagaattgcctttgttattttttcttcctgctgtcaggaacattttgaataccagagaaaaagaaaagtgctcttcttggcatgggaggagttgtcacacttgcaaaataaaggatgcagtcccaaatgttcataatctcagggtctgaaggaggatcagaaactgtgtatacaatttcaggcttctctgaatgcagcttttgaaagctgttcctggccgaggcagtactagtcagaaccctcggaaacaggaacaaatgtcttcaaggtgcagcaggaggaaacaccttgcccatcatgaaagtgaataaccactgccgctgaaggaatccagctcctgtttgagcaggtgctgcacactcccacactgaaacaacagttcatttttataggacttccaggaaggatcttcttcttaagcttcttaattatggtacatctccagttggcagatgactatgactactgacaggagaatgaggaactagctgggaatatttctgtttgaccaccatggagtcacccatttctttactggtatttggaaataataattctgaattgcaaagcaggagttagcgaagatcttcatttcttccatgttggtgacagcacagttctggctatgaaagtctgcttacaaggaagaggataaaaatcatagggataataaatctaagtttgaagacaatgaggttttagctgcatttgacatgaagaaattgagacctctactggatagctatggtatttacgtgtctttttgcttagttacttattgaccccagctgaggtcaagtatgaactcaggtctctcgggctactggcatggattgattacatacaactgtaattttagcagtgatttagggtttatgagtacttttgcagtaaatcatagggttagtaatgttaatctcagggaaaaaaaaaaaaagccaaccctgacagacatcccagctcaggtggaaatcaaggatcacagctcagtgcggtcccagagaacacagggactcttctcttaggacctttatgtacagggcctcaagataactgatgttagtcagaagactttccattctggccacagttcagctgaggcaatcctggaattttctctccgctgcacagttccagtcatcccagtttgtacagttctggcactttttgggtcaggccgtgatccaaggagcagaagttccagctatggtcagggagtgcctgaccgtcccaactcactgcactcaaacaaaggcgaaaccacaagagtggcttttgttgaaattgcagtgtggcccagaggggctgcaccagtactggattgaccacgaggcaacattaatcctcagcaagtgcaatttgcagccattaaattgaactaactgatactacaatgcaatcagtatcaacaagtggtttggcttggaagatggagtctaggggctctacaggagtagctactctctaatggagttgcattttgaagcaggacactgtgaaaagctggcctcctaaagaggctgctaaacattagggtcaattttccagtgcactttctgaagtgtctgcagttccccatgcaaagctgcccaaacatagcacttccaattgaatacaattatatgcaggcgtactgcttcttgccagcactgtccttctcaaatgaactcaacaaacaatttcaaagtctagtagaaagtaacaagctttgaatgtcattaaaaagtatatctgctttcagtagttcagcttatttatgcccactagaaacatcttgtacaagctgaacactggggctccagattagtggtaaaacctactttatacaatcatagaatcatagaatggcctgggttggaagggaccccaaggatcatgaagatccaacacccccgccacaggcagggccaccaacctccagatctggtactagaccaggcagcccagggctccatccaacctggccatgaacacctccagggatggagcatccacaacctctctgggcagcctgtgccagcacctcaccaccctctctgtgaagaacttttccctgacatccaatctaagccttccctccttgaggttagatccactcccccttgtgctatcactgtctactcttgtaaaaagttgattctcctcctttttggaaggttgcaatgaggtctccttgcagccttcttctcttctgcaggatgaacaagcccagctccctcagcctgtctttataggagaggtgctccagccctctgatcatctttgtggccctcctctggacccgctccaagagctccacatctttcctgtactgggggccccaggcctgaatgcagtactccagatggggcctcaaaagagcagagtaaagagggacaatcaccttcctcaccctgctggccagccctcttctgatggagccctggatacaactggctttctgagctgcaacttctccttatcagttccactattaaaacaggaacaatacaacaggtgctgatggccagtgcagagtttttcacacttcttcatttcggtagatcttagatgaggaacgttgaagttgtgcttctgcgtgtgcttcttcctcctcaaatactcctgcctgatacctcaccccacctgccactgaatggctccatggccccctgcagccagggccctgatgaacccggcactgcttcagatgctgtttaatagcacagtatgaccaagttgcacctatgaatacacaaacaatgtgttgcatccttcagcacttgagaagaagagccaaatttgcattgtcaggaaatggtttagtaattctgccaattaaaacttgtttatctaccatggctgtttttatggctgttagtagtggtacactgatgatgaacaatggctatgcagtaaaatcaagactgtagatattgcaacagactataaaattcctctgtggcttagccaatgtggtacttcccacattgtataagaaatttggcaagtttagagcaatgtttgaagtgttgggaaatttctgtatactcaagagggcgtttttgacaactgtagaacagaggaatcaaaagggggtgggaggaagttaaaagaagaggcaggtgcaagagagcttgcagtcccgctgtgtgtacgacactggcaacatgaggtctttgctaatcttggtgctttgcttcctgcccctggctgccttagggtgcgatctgcctcagacccacagcctgggcagcaggaggaccctgatgctgctggctcagatgaggagaatcagcctgtttagctgcctgaaggataggcacgattttggctttcctcaagaggagtttggcaaccagtttcagaaggctgagaccatccctgtgctgcacgagatgatccagcagatctttaacctgtttagcaccaaggatagcagcgctgcttgggatgagaccctgctggataagttttacaccgagctgtaccagcagctgaacgatctggaggcttgcgtgatccagggcgtgggcgtgaccgagacccctctgatgaaggaggatagcatcctggctgtgaggaagtactttcagaggatcaccctgtacctgaaggagaagaagtacagcccctgcgcttgggaagtcgtgagggctgagatcatgaggagctttagcctgagcaccaacctgcaagagagcttgaggtctaaggagtaaaaagtctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgctctagaactagtggatcccccggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatctgcgatctaagtaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcaggtaccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatg

SEQ ID NO: 8 - DNA, 17402 bp, Artificial Sequence
  Plasmid pOMIFN-Ins-CMV-pur-attB
ggccgccaccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctgggtaccgggccccccctcgactagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggctctagtcgaggtcgacggtatcgataagcttgattaggcagagcaataggactctcaacctcgtgagtatggcagcatgttaactctgcactggagtccagcgtgggaaacaatctgccttgcacatgagtcttcgtgggccaatattccccaacggttttccttcagcttgtcttgtctcctaagctctcaaaacacctttttggtgaataaactcacttggcaacgtttatctgtcttaccttagtgtcacgtttcatccctattcccctttctcctcctccgtgtggtacacagtggtgcacactggttcttctgttgatgttctgctctgacagccaatgtgggtaaagttcttcctgccacgtgtctgtgttgttttcacttcaaaaagggccctgggctccccttggagctctcaggcatttccttaatcatcacagtcacgctggcaggattagtccctcctaaaccttagaatgacctgaacgtgtgctccctctttgtagtcagtgcagggagacgtttgcctcaagatcagggtccatctcacccacagggccattcccaagatgaggtggatggtttactctcacaaaaagttttcttatgtttggctagaaaggagaactcactgcctacctgtgaattcccctagtcctggttctgctgccactgctgcctgtgcagcctgtcccatggagggggcagcaactgctgtcacaaaggtgatcccaccctgtctccactgaaatgacctcagtgccacgtgttgtatagggtataaagtacgggagggggatgcccggctcccttcagggttgcagagcagaagtgtctgtgtatagagtgtgtcttaatctattaatgtaacagaacaacttcagtcctagtgttttgtgggctggaattgcccatgtggtagggacaggcctgctaaatcactgcaatcgcctatgttctgaaggtatttgggaaagaaagggatttgggggattgcctgtgattggctttaattgaatggcaaatcacaggaaagcagttctgctcaacagttggttgtttcagccaattcttgcagccaaagagccgggtgcccagcgatataatagttgtcacttgtgtctgtatggatgacagggaggtagggtgacctgaggaccaccctccagcttctgctagcgtaggtacagtcaccacctccagctccacacgagtcccatcgtggtttaccaaagaaacacaattatttggaccagtttggaaagtcacccgctgaattgtgaggctagattaatagagctgaagagcaaatgttcccaacttggagatactagttggtattagtatcagaggaacagggccatagcacctccatgctattagattccggctggcatgtacttttcaagatgatttgtaactaacaatggcttattgtgcttgtcttaagtctgtgtcctaatgtaaatgttcctttggtttatataaccttcttgccatttgctcttcaggtgttcttgcagaacactggctgctttaatctagtttaactgttgcttgattattcttagggataagatctgaataaactttttgtggctttggcagactttagcttgggcttagctcccacattagcttttgctgccttttctgtgaagctatcaagatcctactcaatgacattagctgggtgcaggtgtaccaaatcctgctctgtggaacacattgtctgatgataccgaaggcaaacgtgaactcaaagaggcacagagttaagaagaagtctgtgcaattcagaggaaaagccaaagtggccattagacacactttccatgcagcatttgccagtaggtttcatataaaactacaaaatggaataaaccactacaaatgggaaaagcctgatactagaatttaaatattcacccaggctcaaggggtgtttcatggagtaatatcactctataaaagtagggcagccaattattcacagacaaagcttttttttttctgtgctgcagtgctgtttttcggctgatccagggttacttattgtgggtctgagagctgaatgatttctccttgtgtcatgttggtgaaggagatatggccagggggagatgagcatgttcaagaggaaacgttgcattttggtggcttgggagaaaggtagaacgatatcaggtccatagtgtcactaagagatctgaaggatggttttacagaacagttgacttggctgggtgcaggcttggctgtaaatggatggaaggatggacagatgggtggacagagatttctgtgcaggagatcatctcctgagctcggtgcttgacagactgcagatccatcccataaccttctccagcatgagagcgcggggagctttggtactgttcagtctgctgcttgttgcttcctgggtgcacagtggtgattttcttactcacacagggcaaaaacctgagcagcttcaaagtgaacaggttgctctcataggccattcagttgtcaagatgaggtttttggtttcttgttttgtaaggtgggaagaagcactgaaggatcagttgcgagggcaggggtttagcactgttcagagaagtcttattttaactcctctcatgaacaaaaagagatgcaggtgcagattctggcaagcatgcagtgaaggagaaagccctgaatttctgatatatgtgcaatgttgggcacctaacattccccgctgaagcacagcagctccagctccatgcagtactcacagctggtgcagccctcggctccagggtctgagcagtgctgggactcacgaggttccatgtctttcacactgataatggtccaatttctggaatgggtgcccatccttggaggtccccaaggccaggctggctgcgtctccgagcagcccgatctggtggtgagtagccagcccatggcaggagttagagcctgatggtctttaaggtcccttccaacctaagccatcctacgattctaggaatcatgacttgtgagtgtgtattgcagaggcaatattttaaagttataaatgttttctccccttccttgtttgtcaaagttatcttgatcgccttatcaatgcttttggagtctccagtcatttttcttacamcaaaaagaggaggaagaatgaagagaatcatttaatttcttgattgaatagtaggattcagaaagctgtacgtaatgccgtctctttgtatcgagctgtaaggtttctcatcatttatcagcgtggtacatatcagcacttttccatctgatgtggaaaaaaaaatccttatcatctacagtctctgtacctaaacatcgctcagactctttaccaaaaaagctataggttttaaaactacatctgctgataatttgccttgttttagctcttcttccatatgctgcgtttgtgagaggtgcgtggatgggcctaaactctcagctgctgagcttgatgggtgcttaagaatgaagcactcactgctgaaactgttttcatttcacaggaatgttttagtggcattgtttttataactacatattcctcagataaatgaaatccagaaataattatgcaaactcactgcatccgttgcacaggtctttatctgctagcaaaggaaataatttggggatggcaaaaacattccttcagacatctatatttaaaggaatataatcctggtacccacccacttcatccctcattatgttcacactcagagatactcattctcttgttgttatcatttgatagcgttttctttggttctttgccacgctctgggctatggctgcacgctctgcactgatcagcaagtagatgcgagggaagcagcagtgagaggggctgccctcagctggcacccagccgctcagcctaggaggggaccttgcctttccaccagctgaggtgcagccctacaagcttacacgtgctgcgagcaggtgagcaaagggagtcttcatggtgtgtttcttgctgcccggaagcaaaactttactttcattcattccccttgaagaatgaggaatgtttggaaacggactgctttacgttcaatttctctcttccctttaaggctcagccaggggccattgctgaggacggcatcggggccccctggaccaaatctgtggcacagatggtttcacttacatcagtggatgtgggatctgcgcctgtaatgtgtccttctgaaggaaggaacgtgccttccaagtgccagccccacagcccccagcccctccctgtgctgctccaattcatctcctcttcctccttctccctttgctgtttgtgctcgggtagaaatcatgaagatttagaagagaaaacaaaataactggagtggaaacccaggtgatgcagttcattcagctgtcataggtttgtcgttgctataggtctgtatcagagatgctarcaccactttgctgtcggtgcttaactcgggtgaactctccttcactcgcatcatttgcgggccttatttacatccccagcatccatcaccctctgggaaaatgggcgcactggatctctaatggaagactttccctctttcagagcctgtgggatgtgcagtgacaagaaacgtggaggggctgagcagcagcactgcccccagggagcaggagcggatgccatcggtggcagcatcccaaatgatgtcagcggatgctgagcaggcagcggacgaacggacagaagcgatgcgtacaccttctgttgacatggtatttggcagcgatttaacactcgcttcctagtcctgctattctccacaggctgcattcaaatgaacgaagggaagggaggcaaaaagatgcaaaatccgagacaagcagcagaaatatttcttcgctacggaagcgtgcgcaaacaaccttctccaacagcaccagaagagcacagcgtaacctttttcaagaccagaaaaggaaattcacaaagcctctgtggataccagcgcgttcagctctcctgatagcagatttcttgtcaggttgcgaatggggtatggtgccaggaggtgcagggaccatatgatcatatacagcacagcagtcattgtgcatgtattaatatatattgagtagcagtgttactttgccaaagcaatagttcagagatgagtcctgctgcatacctctatcttaaaactaacttataaatagtaaaaccttctcagttcagccacgtgctcctctctgtcagcaccaatggtgcttcgcctgcacccagctgcaaggaatcagcccgtgatctcattaacactcagctctgcaggataaattagattgttccactctcttttgttgttaattacgacggaacaattgttcagtgctgatggtcctaattgtcagctacagaaaacgtctccatgcagttccttctgcgccagcaaactgtccaggctatagcaccgtgatgcatgctacctctcactccatccttcttctctttcccaccagggagagctgtgtgttttcactctcagccactctgaacaataccaaactgctacgcactgcctccctcggaaagagaatccccttgttgcttttttatttacaggatccttcttaaaaagcagaccatcattcactgcaaacccagagcttcatgcctctccttccacaaccgaaaacagccggcttcatttgtcttttttaaatgctgttttccaggtgaattttggccagcgtgttggctgagatccaggagcacgtgtcagctttctgctctcattgctcctgttctgcattgcctctttctggggtttccaagagggggggagactttgcgcggggatgagataatgccccttttcttagggtggctgctgggcagcagagtggctctgggtcactgtggcaccaatgggaggcaccagtgggggtgtgttttgtgcaggggggaagcattcacagaatggggctgatcctgaagcttgcagtccaaggctttgtctgtgtacccagtgaaatccttcctctgttacataaagcccagataggactcagaaatgtagtcattccagcccccctcttcctcagatctggagcagcacttgtttgcagccagtcctccccaaaatgcacagacctcgccgagtggagggagatgtaaacagcgaaggttaattacctccttgtcaaaaacactttgtggtccatagatgtttctgtcaatcttacaaaacagaaccgagaggcagcgagcactgaagagcgtgttcccatgctgagttaatgagacttggcagctcgctgtgcagagatgatccctgtgcttcatgggaggctgtaacctgtctccccatcgccttcacaccgcagtgctgtcctggacacctcaccctccataagctgtaggatgcagctgcccagggatcaagagacttttcctaaggctcttaggactcatctttgccgctcagtagcgtgcagcaattactcatcccaactatactgaatgggtttctgccagctctgcttgtttgtcaataagcatttcttcattttgcctctaagtttctctcagcagcaccgctctgggtgacctgagtggccacctggaacccgaggggcacagccaccacctccctgttgctgctgctccagggactcatgtgctgctggatggggggaagcatgaagttcctcacccagacacctgggttgcaatggctgcagcgtgctcttcttggtatgcagattgtttccagccattacttgtagaaatgtgctgtggaagccctttgtatctctttctgtggcccttcagcaaaagctgtgggaaagctctgaggctgctttcttgggtcgtggaggaattgtatgttccttctttaacaaaaattatccttaggagagagcactgtgcaagcattgtgcacataaaacaattcaggttgaaagggctctctggaggtttccagcctgactactgctcgaagcaaggccaggttcaaagatggctcaggatgctgtgtgccttcctgattatctgtgccaccaatggaggagattcacagccactctgcttcccgtgccactcatggagaggaatattcccttatattcagatagaatgttatcctttagctcagccttccctataaccccatgagggagctgcagatccccatactctccccttctctggggtgaaggccgtgtcccccagccccccttcccaccctgtgccctaagcagcccgctggcctctgctggatgtgtgcctatatgtcaatgcctgtccttgcagtccagcctgggacatttaattcatcaccagggtaatgtggaactgtgtcatcttcccctgcagggtacaaagttctgcacggggtcctttcggttcaggaaaaccttcactggtgctacctgaatcaagctctatttaataagttcataagcacatggatgtgttttcctagagatacgttttaatggtatcagtgatttttatttgctttgttgcttacttcaaacagtgcctttgggcaggaggtgagggacgggtctgccgttggctctgcagtgatttctccaggcgtgtggctcaggtcagatagtggtcactctgtggccagaagaaggacaaagatggaaattgcagattgagtcacgttaagcaggcatcttggagtgatttgaggcagtttcatgaaagagctacgaccacttattgttgttttccccttttacaacagaagttttcatcaaaataacgtggcaaagcccaggaatgtttgggaaaagtgtagttaaatgttttgtaattcatttgtcggagtgctaccagctaagaaaaaagtcctacctttggtatggtagtcctgcagagaatacaacatcaatattagtttggaaaaaaacaccaccaccaccagaaactgtaatggaaaatgtaaaccaagaaattccttgggtaagagagaaaggatgtcgtatactggccaagtcctgcccagctgtcagcctgctgaccctctgcagttcaggaccatgaaacgtggcactgtaagacgtgtcccctgcctttgcttgcccacagatctctgcccttgtgctgactcctgcacacaagagcatttccctgtagccaaacagcgattagccataagctgcacctgactttgaggattaagagtttgcaattaagtggattgcagcaggagatcagtggcagggttgcagatgaaatccttttctaggggtagctaagggctgagcaacctgtcctacagcacaagccaaaccagccaagggttttcctgtgctgttcacagaggcagggccagctggagctggaggaggttgtgctgggacccttctccctgtgctgagaatggagtgatttctgggtgctgttcctgtggcttgcactgagcagctcaagggagatcggtgctcctcatgcagtgccaaaactcgtgtttgatgcagaaagatggatgtgcacctccctcctgctaatgcagccgtgagcttatgaaggcaatgagccctcagtgcagcaggagctgtagtgcactcctgtaggtgctagggaaaatctctggttcccagggatgcattcataagggcaatatatcttgaggctgcgccaaatctttctgaaatattcatgcgtgttcccttaatttatagaaacaaacacagcagaataattattccaatgcctcccctcgaaggaaacccatatttccatgtagaaatgtaacctatatacacacagccatgctgcatccttcagaacgtgccagtgctcatctcccatggcaaaatactacaggtattctcactatgttggacctgtgaaaggaaccatggtaagaaacttcggttaaaggtatggctgcaaaactactcataccaaaacagcagagctccagacctcctcttaggaaagagccacttggagagggatggtgtgaaggctggaggtgagagacagagcctgtcccagttttcctgtctctattttctgaaacgtttgcaggaggaaaggacaactgtactttcaggcatagctggtgccctcacgtaaataagttccccgaacttctgtgtcatttgttcttaagatgctttggcagaacactttgagtcaattcgcttaactgtgactaggtctgtaaataagtgctccctgctgataaggttcaagtgacatttttagtggtatttgacagcatttaccttgctttcaagtcttctaccaagctcttctatacttaagcagtgaaaccgccaagaaacccttccttttatcaagctagtgctaaataccattaacttcataggttagatacggtgctgccagcttcacctggcagtggttggtcagttctgctggtgacaaagcctccctggcctgtgcttttacctagaggtgaatatccaagaatgcagaactgcatggaaagcagagctgcaggcacgatggtgctgagccttagctgcttcctgctgggagatgtggatgcagagacgaatgaaggacctgtcccttactcccctcagcattctgtgctatttagggttctaccagagtccttaagaggttttttttttttttggtccaaaagtctgtttgtttggttttgaccactgagagcatgtgacacttgtctcaagctattaaccaagtgtccagccaaaatcaattgcctgggagacgcagaccattacctggaggtcaggacctcaataaatattaccagcctcattgtgccgctgacagattcagctggctgctccgtgttccagtccaacagttcggacgccacgtttgtatatatttgcaggcagcctcggggggaccatctcaggagcagagcaccggcagccgcctgcagagccgggcagtacctcaccatggctttgacctttgccttactggtggctctcctggtgctgagctgcaagagcagctgctctgtgggctgcgatctgcctcagacccacagcctgggcagcaggaggaccctgatgctgctggctcagatgaggagaatcagcctgtttagctgcctgaaggataggcacgattttggctttcctcaagaggagtttggcaaccagtttcagaaggctgagaccatccctgtgctgcacgagatgatccagcagatctttaacctgtttagcaccaaggatagcagcgctgcttgggatgagaccctgctggataagttttacaccgagctgtaccagcagctgaacgatctggaggcttgcgtgatccagggcgtgggcgtgaccgagacccctctgatgaaggaggatagcatcctggctgtgaggaagtactttcagaggatcaccctgtacctgaaggagaagaagtacagcccctgcgcttgggaagtcgtgagggctgagatcatgaggagctttagcctgagcaccaacctgcaagagagcttgaggtctaaggagtaaaaagtctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataccgtcgacctcgactagagcggccactaacatacgctctccatcaaaacaaaacgaaacaaaacaaactagcaaaataggctgtccccagtgcaagtgcaggtgccagaacatttctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatctgcgatctaagtaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcaggtaccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagc

SEQ ID NO: 9 - DNA, 5172 bp, Artificial Sequence
  Plasmid pRSV-Int
ctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgctaggggtctaggatcgattctaggaattctctagccgcggtctagggatcccggcgcgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagcttgcatgcctgcaggtaccggtccggaattcccgggtcgacgagctcactagtcgtagggtcgccgacatgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtagcgagacacccggatccctcgaggggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaggtgcccagtcatagccgaatagcctctccacccaagcggccggagaacctgcgtgcaatccactgggggcgcg

SEQ ID NO: 10 - DNA, 6233 bp, Artificial Sequence
  Plasmid pCR-XL-TOPO-CMV-pur-attB
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacgcgttagaatactcaagctatgcatcaagcttggtaccgagctcggatccactagtaacggccgccagtgtgctggaattcgcccttggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgctagccctcgagcaggatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctcgactctaggggctcgagatctgcgatctaagtaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaattcggcttcaggtaccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacagccgaattgatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 11 - DNA, 234 bp, Artificial Sequence
  attP containing polynucleotide
gactagtactgacggacacaccgaagccccggcggcaaccctcagcggatgccccggggcttcacgttttcccaggtcagaagcggttttcgggagtagtgccccaactggggtaacctttgagttctctcagttgggggcgtagggtcgccgacatgacacaaggggttgtgaccggggtggacacgtacgcgggtgcttacgaccgtcagtcgcgcgagcgcgactagtaca

SEQ ID NO: 12 - DNA, 26 bp, Artificial Sequence
  Primer attB-for
taccgtcgacgatgtaggtcacggtc

SEQ ID NO: 13 - protein, 11 aa, SV40
CGGPKKKRKVG

SEQ ID NO: 14 - DNA, 237 bp, mouse
gacctggaatatcgcgagtaaactgaaaatcacggaaaatgagaaatacacactttaggacgtgaaatatggcgaggaaaactgaaaaaggtggaaaatttagaaatgtccactgtaggacgtggaatatggcaagaaaactgaaaatcatggaaaatgagaaacatccacttgacgacttgaaaaatgacgaaatcactaaaaaacgtgaaaaatgagaaatgcacactgaaggac

SEQ ID NO: 15 - DNA, 275 bp, mouse
gagtgagttacactgaaaaacacatacgttggaaaccggcattgtagaacagtgtatatcaatgagttacaatgagaaaaatggaaaatgataaaaaccacagtgtagaacatattagatgtgtgagttacactgaaaaacacattccttggaaacgggatttgtagaactgtgtatatcaatgagttacaatgagaaacatggaaaatgataaaaaccacactgtagaacattttagatgagtgagttacactgaaaaacacatatgttggaaa

SEQ ID NO: 16 - DNA, 9299 bp, mouse
ggccgctcttctcgttctgccagcgggccctcgtctctccaccccatccgtctgccggtggtgtgtggaaggcaggggtgcggctctccggcccgacgctgccccgcgcgcacttttctcagtggttcgcgtggtccttgtggatgtgtgaggcgcccggttgtgccctcacgtgtttcactttggtcgtgtctcgcttgaccatgttcccagagtcggtggatgtggccggtggcgttgcatacccttcccgtctggtgtgtgcacgcgctgtttcttgtaagcgtcgaggtgctcctggagcgttccaggtttgtctcctaggtgcctgcttctgagctggtggtggcgctccccattccctggtgtgcctccggtgctccgtctggctgtgtgccttcccgtttgtgtctgagaagcccgtgagaggggggtcgaggagagaaggaggggcaagaccccccttcttcgtcgggtgaggcgcccaccccgcgactagtacgcctgtgcgtagggctggtgctgagcggtcgcggctggggttggaaagtttctcgagagactcattgctttcccgtggggagctttgagaggcctggctttcgggggggaccggttgcagggtctcccctgtccgcggatgctcagaatgcccttggaagagaaccttcctgttgccgcagacccccccgcgcggtcgcccgcgtgttggtcttctggtttccctgtgtgctcgtcgcatgcatcctctctcggtggccggggctcgtcggggttttgggtccgtcccgccctcagtgagaaagtttccttctctagctatcttccggaaagggtgcgggcttcttacggtctcgaggggtctctcccgaatggtcccctggagggctcgccccctgaccgcctcccgcgcgcgcagcgtttgctctctcgtctaccgcggcccgcggcctccccgctccgagttcggggagggatcacgcggggcagagcctgtctgtcgtcctgccgttgctgcggagcatgtggctcggcttgtgtggttggtggctggggagagggctccgtgcacacccccgcgtgcgcgtactttcctcccctcctgagggccgccgtgcggacggggtgtgggtaggcgacggtgggctcccgggtccccacccgtcttcccgtgcctcacccgtgccttccgtcgcgtgcgtccctctcgctcgcgtccacgactttggccgctcccgcgacggcggcctgcgccgcgcgtggtgcgtgctgtgtgcttctcgggctgtgtggttgtgtcgcctcgccccccccttcccgcggcagcgttcccacggctggcgaaatcgcgggagtcctccttcccctcctcggggtcgagagggtccgtgtctggcgttgattgatctcgctctcggggacgggaccgttctgtgggagaacggctgttggccgcgtccggcgcgacgtcggacgtggggacccactgccgctcgggggtcttcgtcggtaggcatcggtgtgtcggcatcggtctctctctcgtgtcggtgtcgcctcctcgggctcccggggggccgtcgtgtttcgggtcggctcggcgctgcaggtgtggtgggactgctcaggggagtggtgcagtgtgattcccgccggttttgcctcgcgtgccctgaccggtccgacgcccgagcggtctctcggtcccttgtgaggacccccttccgggaggggcccgtttcggccgcccttgccgtcgtcgccggccctcgttctgctgtgtcgttcccccctccccgctcgccgcagccggtcttttttcctctctccccccctctcctctgactgacccgtggccgtgctgtcggaccccccgcatgggggcggccgggcacgtacgcgtccgggcggtcaccggggtcttgggggggggccgaggggtaagaaagtcggctcggcgggcgggaggagctgtggtttggagggcgtcccggccccgcggccgtggcggtgtcttgcgcggtcttggagagggctgcgtgcgaggggaaaaggttgccccgcgagggcaaagggaaagaggctagcagtggtcattgtcccgacggtgtggtggtctgttggccgaggtgcgtctggggggctcgtccggccctgtcgtccgtcgggaaggcgcgtgttggggcctgccggagtgccgaggtgggtaccctggcggtgggattaaccccgcgcgcgtgtcccggtgtggcggtgggggctccggtcgatgtctacctccctctccccgaggtctcaggccttctccgcgcgggctctcggccctcccctcgttcctccctctcgcggggttcaagtcgctcgtcgacctcccctcctccgtccttccatctctcgcgcaatggcgccgcccgagttcacggtgggttcgtcctccgcctccgcttctcgccgggggctggccgctgtccggtctctcctgcccgacccccgttggcgtggtcttctctcgccggcttcgcggactcctggcttcgcccggagggtcagggggcttcccggttccccgacgttgcgcctcgctgctgtgtgcttggggggggcccgctgcggcctccgcccgcccgtgagcccctgccgcacccgccggtgtgcggtttcgcgccgcggtcagttgggccctggcgttgtgtcgcgtcgggagcgtgtccgcctcgcggcggctagacgcgggtgtcgccgggctccgacgggtggcctatccagggctcgcccccgccgacccccgcctgcccgtcccggtggtggtcgttggtgtggggagtgaatggtgctaccggtcattccctcccgcgtggtttgactgtctcgccggtgtcgcgcttctctttccgccaacccccacgccaacccaccaccctgctctcccggcccggtgcggtcgacgttccggctctcccgatgccgaggggttcgggatttgtgccggggacggaggggagagcgggtaagagaggtgtcggagagctgtcccggggcgacgctcgggttggctttgccgcgtgcgtgtgctcgcggacgggttttgtcggaccccgacggggtcggtccggccgcatgcactctcccgttccgcgcgagcgcccgcccggctcacccccggtttgtcctcccgcgaggctctccgccgccgccgcctcctcctcctctctcgcgctctctgtcccgcctggtcctgtcccacccccgacgctccgctcgcgcttccttacctggttgatcctgccaggtagcatatgcttgtctcaaagattaagccatgcatgtctaagtacgcacggccggtacagtgaaactgcgaatggctcattaaatcagttatggttcctttggtcgctcgctcctctcctacttggataactgtggtaattctagagctaatacatgccgacgggcgctgaccccccttcccggggggggatgcgtgcatttatcagatcaaaaccaacccggtgagctccctcccggctccggccgggggtcgggcgccggcggcttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagtcaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgactggcgatgcggcggcgttattcccatgacccgccgggcagcttccgggaaaccaaagtctttgggttccggggggagtatggttgcaaagctgaaacttaaaggaattgacggaagggcaccaccaggagtgggcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctgcgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcattaaacgggagactgtggaggagcggcggcgtggcccgctctccccgtcttgtgtgtgtcctcgccgggaggcgcgtgcgtcccgggtcccgtcgcccgcgtgtggagcgaggtgtctggagtgaggtgagagaaggggtgggtggggtcggtctgggtccgtctgggaccgcctccgatttcccctccccctcccctctccctcgtccggctctgacctcgccaccctaccgcggcggcggctgctcgcgggcgtcttgcctctttcccgtccggctcttccgtgtctacgaggggcggtacgtcgttacgggtttttgacccgtcccgggggcgttcggtcgtcggggcgcgcgctttgctctcccggcacccatccccgccgcggctctggcttttctacgttggctggggcggttgtcgcgtgtggggggatgtgagtgtcgcgtgtgggctcgcccgtcccgatgccacgcttttctggcctcgcgtgtcctccccgctcctgtcccgggtacctagctgtcgcgttccggcgcggaggtttaaggaccccgggggggtcgccctgccgcccccagggtcggggggcggtggggcccgtagggaagtcggtcgttcgggcggctctccctcagactccatgaccctcctccccccgctgccgccgttcccgaggcggcggtcgtgtgggggggtggatgtctggagccccctcgggcgccgtgggggcccgacccgcgccgccggcttgcccgatttccgcgggtcggtcctgtcggtgccggtcgtgggttcccgtgtcgttcccgtgtttttccgctcccgaccctttttttttcctcccccccacacgtgtctcgtttcgttcctgctggccggcctgaggctacccctcggtccatctgttctcctctctctccggggagaggagggcggtggtcgttgggggactgtgccgtcgtcagcacccgtgagttcgctcacacccgaaataccgatacgactcttagcggtggatcactcggctcgtgcgtcgatgaagaacgcagctagctgcgagaattaatgtgaattgcaggacacattgatcatcgacacttcgaacgcacttgcggccccgggttcctcccggggctacgcctgtctgagcgtcggttgacgatcaatcgcgtcacccgctgcggtgggtgctgcgcggctgggagtttgctcgcagggccaaccccccaacccgggtcgggccctccgtctcccgaagttcagacgtgtgggcggttgtcggtgtggcgcgcgcgcccgcgtcgcggagcctggtctcccccgcgcatccgcgctcgcggcttcttcccgctccgccgttcccgccctcgcccgtgcaccccggtcctggcctcgcgtcggcgcctcccggaccgctgcctcaccagtctttctcggtcccgtgccccgtgggaacccaccgcgcccccgtggcgcccgggggtgggcgcgtccgcatctgctctggtcgaggttggcggttgagggtgtgcgtgcgccgaggtggtggtcggtcccctgcggccgcggggttgtcggggtggcggtcgacgagggccggtcggtcgcctgcggtggttgtctgtgtgtgtttgggtcttgcgctgggggaggcggggtcgaccgctcgcggggttggcgcggtcgcccggcgccgcgcaccctccggcttgtgtggagggagagcgagggcgagaacggagagaggtggtatccccggtggcgttgcgagggagggtttggcgtcccgcgtccgtccgtccctccctccctcggtgggcgccttcgcgccgcacgcggccgctaggggcggtcggggcccgtggcccccgtggctcttcttcgtctccgcttctccttcacccgggcggtacccgctccggcgccggcccgcgggacgccgcggcgtccgtgcgccgatgcgagtcacccccgggtgttgcgagttcggggagggagagggcctcgctgacccgttgcgtcccggcttccctgggggggacccggcgtctgtgggctgtgcgtcccgggggttgcgtgtgagtaagatcctccacccccgccgccctcccctcccgccggcctctcggggaccccctgagacggttcgccggctcgtcctcccgtgccgccgggtgccgtctctttcccgcccgcctcctcgctctcttcttcccgcggctgggcgcgtgtcccccctttctgaccgcgacctcagatcagacgtggcgacccgctgaatttaagcatattagtcagcggaggaaaagaaactaaccaggattccctcagtaacggcgagtgaacagggaagagcccagcgccgaatccccgccgcgcgtcgcggcgtgggaaatgtggcgtacggaagacccactccccggcgccgctcgtggggggcccaagtccttctgatcgaggcccagcccgtggacggtgtgaggccggtagcggccccggcgcgccgggctcgggtcttcccggagtcgggttgcttgggaatgcagcccaaagcgggtggtaaactccatctaaggctaaataccggcacgagaccgatagtcaacaagtaccgtaagggaaagttgaaaagaactttgaagagagagttcaagagggcgtgaaaccgttaagaggtaaacgggtggggtccgcgcagtccgcccggaggattcaacccggcggcgcgcgtccggccgtgcccggtggtcccggcggatctttcccgctccccgttcctcccgacccctccacccgcgcgtcgttcccctcttcctccccgcgtccggcgcctccggcggcgggcgcggggggtggtgtggtggtggcgcgcgggcggggccgggggtggggtcggcgggggaccgcccccggccggcgaccggccgccgccgggcgcacttccaccgtggcggtgcgccgcgaccggctccgggacggccgggaaggcccggtggggaaggtggctcggggggggcggcgcgtctcagggcgcgccgaaccacctcaccccgagtgttacagccctccggccgcgctttcgccgaatcccggggccgaggaagccagatacccgtcgccgcgctctccctctccccccgtccgcctcccgggcgggcgtgggggtgggggccgggccgcccctcccacggcgcgaccgctctcccacccccctccgtcgcctctctcggggcccggtggggggcggggcggactgtccccagtgcgccccgggcgtcgtcgcgccgtcgggtcccggggggaccgtcggtcacgcgtctcccgacgaagccgagcgcacggggtcggcggcgatgtcggctacccacccgacccgtcttgaaacacggaccaaggagtctaacgcgtgcgcgagtcaggggctcgtccgaaagccgccgtggcgcaatgaaggtgaagggccccgcccgggggcccgaggtgggatcccgaggcctctccagtccgccgagggcgcaccaccggcccgtctcgcccgccgcgccggggaggtggagcacgagcgtacgcgttaggacccgaaagatggtgaactatgcttgggcagggcgaagccagaggaaactctggtggaggtccgtagcggtcctgacgtgcaaatcggtcgtccgacctgggtataggggcgaaagactaatcgaaccatctagtagctggttccctccgaagtttccctcaggatagctggcgctctcgctcccgacgtacgcagttttatccggtaaagcgaatgattagaggtcttggggccgaaacgatctcaacctattctcaaactttaaatgggtaagaagcccggctcgctggcgtggagccgggcgtggaatgcgagtgcctagtgggccacttttggtaagcagaactggcgctgcgggatgaaccgaacgccgggttaaggcgcccgatgccgacgctcatcagaccccagaaaaggtgttggttgatatagacagcaggacggtggccatggaagtcggaatccgctaaggagtgtgtaacaactcacctgccgaatcaactagccctgaaaatggatggcgctggagcgtcgggcccatacccggccgtcgccgcagtcggaacggaacgggacgggagcg